SEQUENCE LISTING (44 sequences)

SEQ ID NO: 1 - DNA, 17 bp, Artificial
  PRIMER FOR PNUTS GENE AT POSITIONS 1628-1644
gctcatggatacagcct

SEQ ID NO: 2 - DNA, 17 bp, Artificial
  PRIMER FOR PNUTS GENE AT POSITIONS 1850-1866
catgtcggtctgacagt

SEQ ID NO: 3 - DNA, 17 bp, Artificial
  PRIMER FOR PLAU GENE AT POSITIONS 1120-1136
ccactactacggctctg

SEQ ID NO: 4 - DNA, 16 bp, Artificial
  PRIMER FOR PLAU GENE AT POSITIONS 1294-1309
tctcgtgtagacgcct

SEQ ID NO: 5 - DNA, 17 bp, Artificial
  PRIMER FOR TNFSF14 GENE AT POSITIONS 97-113
accgacatcccattcac

SEQ ID NO: 6 - DNA, 19 bp, Artificial
  PRIMER FOR TNFSF14 GENE AT POSITIONS 295-313
accttcgctcttgtatcag

SEQ ID NO: 7 - DNA, 20 bp, Artificial
  PRIMER FOR ADM GENE AT POSITIONS 1051-1070
tgatttctcacggcgtgtca

SEQ ID NO: 8 - DNA, 21 bp, Artificial
  PRIMER FOR ADM GENE AT POSITIONS 1123-1143
caacacgcattgcacttttcc

SEQ ID NO: 9 - DNA, 21 bp, Artificial
  PRIMER FOR C/EPB ALPHA GENE AT POSITIONS 2072- 2092
agcattgcctaggaacacgaa

SEQ ID NO: 10 - DNA, 23 bp, Artificial
  PRIMER FOR C/EPB ALPHA GENE AT POSITIONS 2266- 2288
tcattttggcaagtatccgagaa

SEQ ID NO: 11 - DNA, 17 bp, Artificial
  PRIMER FOR PAI1 GENE AT POSITIONS 1076-1092
accaagagcctctccac

SEQ ID NO: 12 - DNA, 16 bp, Artificial
  PRIMER FOR PAI1 GENE AT POSITIONS 1290-1305
tgaaggcgtctttccc

SEQ ID NO: 13 - DNA, 19 bp, Artificial
  PRIMER FOR 20585 GENE AT POSITIONS 206-224
gcaacaaagcattggagaa

SEQ ID NO: 14 - DNA, 17 bp, Artificial
  PRIMER FOR 20585 GENE AT POSITIONS 396-412
gttgtggaggaggctga

SEQ ID NO: 15 - DNA, 16 bp, Artificial
  PRIMER FOR FUT4 GENE AT POSITIONS 1226-1241
aatctgcgcttctccc

SEQ ID NO: 16 - DNA, 16 bp, Artificial
  PRIMER FOR FUT4 GENE AT POSITIONS 396-412
ccccaagctcagtgtc

SEQ ID NO: 17 - DNA, 16 bp, Artificial
  PRIMER FOR E2F6 GENE AT POSITIONS 81-96
agaccccatcaacgtg

SEQ ID NO: 18 - DNA, 17 bp, Artificial
  PRIMER FOR E2F6 AT POSITIONS 334-350
tcaacgaggtcgattcc

SEQ ID NO: 19 - DNA, 19 bp, Artificial
  PRIMER FOR FADD GENE AT POSITIONS 211-229
ctcaagttcctatgcctcg

SEQ ID NO: 20 - DNA, 18 bp, Artificial
  PRIMER FOR FADD GENE AT POSITIONS 422-439
cacacaggtcttcttccc

SEQ ID NO: 21 - DNA, 16 bp, Artificial
  PRIMERS FOR 18747 AT POSITIONS 206-224
ggcaaagcgtctgaca

SEQ ID NO: 22 - DNA, 17 bp, Artificial
  PRIMER FOR 18747 GENE AT POSITIONS 2349-2365
gttacggacgtttcggg

SEQ ID NO: 23 - DNA, 19 bp, Artificial
  PRIMER FOR 22147 GENE AT POSITION 206-224
gcaacaaagcattggagaa

SEQ ID NO: 24 - DNA, 17 bp, Artificial
  PRIMER FOR 22147 GENE AT POSTITION 396-412
gttgtggaggaggctga

SEQ ID NO: 25 - DNA, 16 bp, Artificial
  PRIMER FOR ZK1 GENE AT POSITION 477-492
ggtgctgggcacaaac

SEQ ID NO: 26 - DNA, 16 bp, Artificial
  PRIMER FOR ZK1 GENE AT POSTION 675-690
gtccatctccacgctg

SEQ ID NO: 27 - DNA, 19 bp, Artificial
  PRIMER FOR KIAA1698 GENE AT POSITION 467-485
tgtgattagtgcatggtcc

SEQ ID NO: 28 - DNA, 18 bp, Artificial
  PRIMER FOR KIAA1698 GENE AT POSITION 635-652
cagctaccaatgagagcc

SEQ ID NO: 29 - DNA, 16 bp, Artificial
  PRIMER FOR CCRL2 AT POSITION 1225-1240
aaagggagaggtgagc

SEQ ID NO: 30 - DNA, 16 bp, Artificial
  PRIMER FOR CCRL2 GENE AT POSITION 1451-1466
ctggaccaagcgtagg

SEQ ID NO: 31 - DNA, 22 bp, Artificial
  PRIMER FOR MYC GENE AT POSITION 500-521
cagctgcttagacgctggattt

SEQ ID NO: 32 - DNA, 22 bp, Artificial
  PRIMER FOR MYC AT POSITION 593-614
accgagtcgtagtcgaggtcat

SEQ ID NO: 33 - DNA, 16 bp, Artificial
  PRIMER FOR MCL-1 GENE AT POSITION 538-553
tcactaccctcgacgc

SEQ ID NO: 34 - DNA, 17 bp, Artificial
  PRIMER FOR MCL-1 AT POSITIONS 736-752
ccttggaaggccgtctc

SEQ ID NO: 35 - DNA, 20 bp, Artificial
  PRIMER FOR PLAU GENE AT POSITIONS 746-765
cactgcttcattgattaccc

SEQ ID NO: 36 - DNA, 18 bp, Artificial
  PRIMER FOR PLAU GENE AT POSITION 959-976
caggcagatggtctgtat

SEQ ID NO: 37 - DNA, 16 bp, Artificial
  PRIMER FOR TNFSF GENE AT POSITION 719-734
tggttcgactgcgtga

SEQ ID NO: 38 - DNA, 18 bp, Artificial
  PRIMER FOR TNFSF14 AT POSITION 950-967
gggttttgccatgttagc

SEQ ID NO: 39 - DNA, 17 bp, Artificial
  PRIMER FOR FADD GENE AT POSITION 1564-1580
gactgctaagtaggggc

SEQ ID NO: 40 - DNA, 17 bp, Artificial
  PRIMER AT FADD GENE AT POSTION 1750-1766
gatacgagatcccgctg

SEQ ID NO: 41 - DNA, 16 bp, Artificial
  PRIMER FOR 18747 GENE AT POSTITION 2813-2828
acattgctacggagcc

SEQ ID NO: 42 - DNA, 16 bp, Artificial
  PRIMER FOR 18747 GENE AT POSITION 3031-3046
tggtgagtgggtcaca

SEQ ID NO: 43 - DNA, 16 bp, Artificial
  PRIMER FOR 22147 GENE AT POSITION 1854-1869
aggcgggcaacactac

SEQ ID NO: 44 - DNA, 17 bp, Artificial
  PRIMER FOR 22147 GENE AT POSITION 2075-2091
agattgcgctattgcac

(19) United States
(12) Patent Application Publication
(10) Pub. No.: US 2006/0204975 A1
(43) Pub. Date: Sep. 14, 2006

(54) Markers for cyclin dependent kinase inhibitors

(75) Inventors: Simon R. Green, Dundee (GB); Sheelagh Frame, Sutton (GB); David Blake, Dundee (GB)

Correspondence Address: PALMER & DODGE, LLP;KATHLEEN M. WILLIAMS, 111 HUNTINGTON AVENUE, BOSTON, MA, 02199, US

(73) Assignee: Cyclacel Limited

(21) Appl. No.: 11/242,244

(22) Filed: Oct. 3, 2005

Related U.S. Application Data
(63) Continuation of application No. PCT/GB04/01334, filed on Mar. 26, 2004.

(30) Foreign Application Priority Data
Apr. 2, 2003 (GB) 0307643.7

(51) Int. Cl. C12Q 1/68; A61K 31/522

(52) U.S. Cl. 435/6; 514/263.35

(57) ABSTRACT

The present invention relates to pharmacodynamic markers for CDKIs including the candidate 2,6,9-tri-substituted purine known as roscovitine. The identity of these markers facilitates the convenient identification of roscovitine-like activity both in vitro and in vivo.

The present invention relates to pharmacodynamic markers for cyclin dependent kinase inhibitors. In particular, the present invention relates to pharmacodynamic markers for the candidate 2,6,9-tri-substituted purine known as roscovitine (CYC 202) and roscovitine-like compounds. The identity of these markers facilitates the convenient identification of roscovitine-like activity both in vitro and in vivo.

A growing family of cyclin dependent kinase inhibitors (CDKI's) have been identified. These inhibitors have varying activities against the multiple CDK family members. Generally, these inhibitors bind to the ATP binding pockets of CDKs.

The 2,6,9-tri-substituted purines are becoming a well studied class of compound showing promise as CDKI's of use in the treatment of proliferative disorders such as cancers, leukemias and glomerular nephritis. Fischer P & Lane D (Curr Med Chem (2000), vol 7, page 1213) provides a detailed review of CDKI's, their origins and described activities. In particular, roscovitine has been shown to inhibit CDK1, CDK2, CDK5, CDK 7 and CDK 9 and to block cell cycle progression in late G1/early S and in M-phase. The compound (R)-2-[(1-ethyl-2-hydroxyethyl)amino]-6-benzylamino-9-isopropylpurine, known as R-roscovitine was first described in WO97/20842 (Meijer L et al) and has since been developed as a promising candidate anti-cancer agent.

In the development of such agents, extensive pharmacokinetic and pharmacodynamic investigations must be undertaken in order to understand the actual mechanism of action upon administration and satisfy the regulatory authority's requirements as to toxicity and dosing. Such analysis is based upon the complex biochemistry of the cell cycle control system and detailed studies undertaken in the pre-clinical phase of drug development to ascertain the particular mode of activity of the candidate drug.

Of particular advantage in the pharmacokinetic and pharmacodynamic investigations is the identity of specific markers of activity for the candidate drug.

The present invention relates to the observation that a number of genes identified in any of FIGS. 1 to 12 act as specific pharmacodynamic (PD) markers, or “biomarkers”, for the activity of the cyclin dependent kinase inhibitor, roscovitine. In particular, the expression of the genes identified is up or down regulated after roscovitine treatment.

In addition, the present invention relates to the observation that a number of genes, expressed as proteins, can act as protein markers which are pharmacodynamic markers for roscovitine activity. Accordingly, the present invention relates to the identification of protein markers as specific pharmacodynamic markers for roscovitine activity and, in particular, the identification of 28 and 14 kDa markers. Suitably these markers are apolipoprotein A1 and transthyretin, respectively. These protein markers can be proteins whose expression is up or down regulated after roscovitine treatment or can be altered post-translationally modified forms, those forms not being detecFigure or being detecFigure to a greater or lesser extent prior to roscovitine treatment.

Accordingly, in a first aspect, there is provided a method of monitoring activity of a CDKI comprising:
a) isolating a sample, a “treated sample”, from a cell, group of cells, an animal model or human, wherein said cell, group of cells, an animal model or human has been treated with CDKI;
b) determining altered expression of at least one of i) a gene identified in any of FIGS. 1 to 12; ii) a 28 kDa protein or iii) a 14 kDa protein in said treated sample as compared to an untreated control sample as an indication of CDKI activity.

Detection of altered expression including gene expression may be performed by any one of the methods known in the art, particularly by microarray analysis, Western blotting or by PCR techniques such as QPCR as described herein. Altered expression may also be detected by analysing protein content of samples using methods such as SELDI-TOF MS as described herein and using further analytical techniques such as 2Dgel electrophoresis. Techniques such as this can be particularly useful for detecting altered expression in the form of alternative post translationally modified forms of a protein.

In one embodiment, “altered expression” is an increase or decrease of gene expression of a gene identified in any of FIGS. 1 to 12. Suitably, the gene identified in FIGS. 1 to 12 is selected from ADM, FADD, PAI1, PLAU, PNUTS, TNFSF14, C/EBP alpha, 20585, FUT4, E2F6, 18747, 22147, ZK1, KIAA1698, CCRL2, myc and mcl-1.

Suitably, the altered expression of at least one of the genes identified in FIGS. 1 to 12 is a decrease in expression compared to the untreated sample. Alternatively, the altered expression is an increase compared to the untreated sample.

When the invention is performed ex vivo for example, in the pharmacodynamic investigation of CDKI's such as roscovitine, it is preferably performed on a group of cells preferably a cell culture. Preferred cell types are selected from colonic tumour cell lines such as HT29, lung tumour cell lines such as A549, renal tumour cell lines such as A498, bladder tumour cell lines such as HT13, breast tumour cell lines such as MCF7, endometrial tumour cell lines such as AN3CA, uterine tumour cell lines such as MESSA DH6 uterine sarcoma cells, hepatic tumour cell lines such as Hep2G, prostate tumour cell lines such as DU145, T cell tumour cell lines such as Cem T cell, pancreatic tumour cell lines such as MiaPaCa2. Alternatively, the cells may be in the form of a histological sample of a tumor biopsy. In another alternative, the cells may be blood cell cultures such as PBMCs.

Suitably, alterations in expression including changes in gene expression are monitored in samples taken from the mammal or human. Suitable samples include tissue samples such as biopsy, blood, urine, buccal scrapes etc. In one embodiment, gene expression is preferably detected in tumour cells, particularly cells derived from a tumour such as breast, lung, gastric, head and neck, colorectal, renal, pancreatic, uterine, hepatic, bladder, endometrial and prostate cancers and leukemias or from blood cells such as lymphocytes and, preferably, peripheral lymphocytes such as PBMC. In another embodiment altered protein expression is detected in serum or plasma samples from a mammal or human.

In these preferred embodiments, the presence of one of ADM, FADD, PAI1, PLAU, PNUTS, TNFSF14, C/EBP alpha, 20585, FUT4, E2F6, 18747, 22147, ZK1, KIAA1698, CCRL2, myc and mcl-1 is preferably detected in tumor cells, particularly cells derived from colonic or lung tumours or from blood cells such as lymphocytes and, preferably, peripheral lymphocytes such as PBMC.

In a preferred embodiment, the group of cells is a cell culture and, preferably, selected from PBMC, HT29, and A549 cells.

In another embodiment, the group of cells is tumor cells, PBMCs or lymphocytes.

Suitably, the sample is blood. Alternatively the sample may be a tumour biopsy such as a sample taken by laser capture microsurgery.

Preferably, the method further comprises extracting RNA from said sample and detecting gene expression by QPCR.

In another embodiment, gene expression is detected by detecting protein products such as, for example, by Western Blot.

In another embodiment, “altered expression” is an altered pattern of protein expression. Suitably, the altered expression is a decrease in a 28 kDa protein. Altered protein expression may also be the presence or absence of one or more post translational modifications of a 28 kDa protein or a 14 kDa protein in the treated sample compared to the untreated control sample. Preferably, the 28 kDa protein is apolipoprotein A1 and the 14 kDa protein is transthyretin.

Suitably, where altered protein expression is detected, the sample is serum, plasma or tissue culture supernatant. Alternatively, the sample may be a tumour biopsy such as a sample taken by laser capture microscopy.

In detection of proteins in serum and, in particular, in plasma samples of patients, samples are removed and subjected to protein analytical techniques such as SELDI-TOF MS, as described herein.

In a preferred embodiment of the method in accordance with any embodiment recited above the CDKI is a compound having roscovitine activity, and preferably is roscovitine or a roscovitine analogue or derivative. Most preferably, roscovitine is R-roscovitine. Suitably, roscovitine is administered to a mammal and, preferably, a human.

In another aspect of the invention there is provided a method of assessing suitable dose levels of roscovitine comprising monitoring the altered expression of at least one of the genes identified in FIGS. 1 to 12 after administration of roscovitine to a cell, group of cells, animal model or human.

In a further aspect, there is provided a method of assessing suitable dose levels of roscovitine comprising monitoring the altered expression of a 28 or 14 kDa protein after administration of roscovitine to a cell, group of cells, animal model or human.

In another aspect there is provided a method for identifying a candidate drug having CDKI-like activity comprising administering said candidate drug to a cell, group of cells, animal model or human and detecting altered expression of at least one of i) a gene identified in any of FIGS. 1 to 12; ii) a 28 kDa protein or iii) a 14 kDa protein in said treated sample as compared to an untreated control sample as an indication of CDKI activity. In this aspect, a candidate drug will show a similar pattern of altered expression of the biomarker to that obtained using a known CDKI.

Use of at least one of the genes as identified in FIGS. 1 to 12 or a gene encoding apolipoprotein A1 or transthyretin in the monitoring of activity of a CDKI, preferably, roscovitine. Suitably, the presence of at least one of the genes as identified in FIGS. 1 to 12 or a 28 or 14 kDa protein is monitored after the administration of a CDKI such as roscovitine to a cell, group of cells, an animal model or human.

Kits for assessing the activity of a CDKI such as roscovitine may be made, comprising nucleic acid primers or antibodies for at least one of the genes or proteins as identified herein. The kits may be used in accordance with any of the hereinbefore described methods for monitoring roscovitine activity, assessing roscovitine dosage or the roscovitine-like activity of a candidate drug.

In a further aspect, there is provided a kit for assessing the activity of a CDKI such as roscovitine comprising antibodies for a protein encoded by at least one of the genes identified in FIGS. 1 to 12, or a 28 or 14 kDa protein. Suitably, such kits may comprise the antibodies recognising the protein product of a gene identified herein alone or in combination with antibodies directed to another gene identified herein.

Antibodies for the genes or proteins identified herein may be derived from commercial sources or through techniques which are familiar to those skilled in the art. In one embodiment, and where altered expression manifests itself through the expression of alteration of post translationally-modified forms of a protein biomarker, antibodies specific for those different forms may be used.

In yet another aspect there is provided a kit for assessing the activity of a CDKI such as roscovitine comprising a probe for detecting gene expression such as a nucleic acid probe for at least one of the genes identified in FIGS. 1 to 12. For example, suitable kits may be kits for QPCR analysis comprising primers for the detection of expression of at least one of the genes identified herein. Examples of suitable primers are set out in FIGS. 13 and 14. Suitably, kits for QPCR analysis may detect at least one gene, and may also comprise primers directed to another gene identified herein. For altered expression detected by analysis of protein samples, a kit may comprise a buffer, chip and quality controls (i.e. known positives or negatives) for detection of a 28 kDa or a 14 kDa protein. Suitable buffers and chips are described herein.

In another aspect there is provided a method of monitoring the activity of a CDKI comprising:

(i) administering said CDKI to a cell, group of cells, an animal model or human; and

(ii) measuring gene expression in samples derived from the treated and the untreated cells, animal or human; and

(iii) detecting an increase or a decrease in gene expression of at least one of the genes identified in any of FIGS. 1 to 12 or a 28 or 14 kDa protein in the treated sample as compared to the untreated sample as an indication of CDKI activity.

In a further aspect, there is provided a method of monitoring the activity of roscovitine comprising:

(i) administering roscovitine to a cell, group of cells, an animal model or human; and

(ii) measuring gene expression in samples derived from the treated and the untreated cells, animal or human; and

(iii) detecting an increase or a decrease in gene expression of at least one of the genes identified in FIGS. 1 to 12 in the treated sample as compared to the untreated sample as an indication of roscovitine activity.

In another aspect there is provided a method according to any preceding claim, wherein the level of at least one of the genes identified in FIGS. 1 to 12 is less than that detected prior to administration of roscovitine.

In a further aspect there is provided a method of assessing suitable dose levels of roscovitine comprising monitoring the degree and rate of expression of at least one of the genes identified in FIGS. 1 to 12 after administration of roscovitine to a cell, group of cells, animal model or human.

In a yet further aspect, there is provided a method of identifying a candidate drug having roscovitine-like activity comprising administering said candidate drug to a cell, group of cells, animal model or human and monitoring the presence or absence of at least one of the genes as identified in FIGS. 1 to 12.

Suitably, a number of the biomarkers of roscovitine activity (i.e. genes identified in any of FIGS. 1 to 12 or expressed as protein markers including apolipoprotein A1 or transthyretin) may be observed in combination.

Preferably, where roscovitine is administered to a human, the effective concentration of roscovitine administered to a cell is greater than 5 micromolar and, more preferably greater than 10 micromolar.

Suitably, where roscovitine is administered to a human, treatment with the drug is for 2, 4 or 8 hours prior to removing blood samples for analysis of gene expression. Where serum or plasma samples are removed for analysis of altered protein expression, roscovitine is administered over a period of days.

In one embodiment, where roscovitine is administered to a cell, the effective concentration of roscovitine is preferably up to 75 micromolar.

In one preferred embodiment, the cell, group of cells, animal model or human, is treated with roscovitine at 7.5, 15 or 30 micromolar for 1.5 hours before analysis to detect gene expression. In this embodiment, a decrease in gene expression of at least one of the genes identified in FIG. 3 or FIG. 7 is detected as an indication of roscovitine activity. In this embodiment, gene expression in cells is preferably detected in PBMC or cells having a phenotype similar to HT29.

In another embodiment, the cell, group of cells, animal model or human, is treated with roscovitine at 7.5, 15 or 30 micromolar for 3 hours before analysis to detect gene expression. In this embodiment, a decrease in gene expression of at least one of the genes identified in FIG. 4 or FIG. 8 is detected as an indication of roscovitine activity. In this embodiment, gene expression in cells is preferably detected in PBMC or cells having a phenotype similar to HT29.

In another embodiment, the cell, group of cells, animal model or human, is treated with roscovitine at 15, 45 or 75 micromolar for 2 hours before analysis to detect gene expression. In this embodiment, a decrease in gene expression of at least one of the genes identified in FIG. 11 is detected as an indication of roscovitine activity. In this embodiment, gene expression in cells is preferably detected in cells having a phenotype similar to A549.

In another embodiment, the cell, group of cells, animal model or human, is treated with roscovitine at 15, 45 or 75 micromolar for 4 hours before analysis to detect gene expression. In this embodiment, a decrease in gene expression of at least one of the genes identified in FIG. 12 is detected as an indication of roscovitine activity. In this embodiment, gene expression in cells is preferably detected in cells having a phenotype similar to A549.

In another embodiment, the cell, group of cells, animal model or human, is treated with roscovitine at 50 micromolar for 4, 12, 24 or 48 hours before analysis to detect gene expression.

In yet another embodiment, where a human is treated, the roscovitine is administered at between 0.8 to 3.6 g per day and, preferably, 1.6 to 2.4 g per day for 1 to 10 days.

As used herein, the term “PBMC” refers to peripheral blood mononuclear cells and includes PBLs (peripheral blood lymphocytes).

In one preferred embodiment, the gene whose expression is detected is selected from ADM, FADD, PAI1, PLAU, PNUTS, TNFSF14, C/EBP alpha, NM_{—}017665 (referred to herein as “20585” which corresponds to NM_{—}017665 Homo sapiens hypothetical protein FLJ20094), FUT4, E2F6, NM_{—}018316 (referred to herein as “18747” which corresponds to NM_{—}018316 Homo sapiens hypothetical protein FLJ1078), NM_{—}033410 (referred to herein as “22147” which corresponds to NM_{—}033410 Homo sapiens hypothetical protein MGC13138), ZK1, KIAA1698, CCRL2, myc and mcl-1.

As used herein the terms “roscovitine” and “R-roscovitine” is used to refer to the compound 2-(R)-(1-ethyl-2-hydroxyethylamino)-6-benzylamino-9-isopropylpurine, also referred to as CYC202. In its unqualified form the term “roscovitine” is used to include the R-roscovitine, the S enantiomer and racemic mixtures thereof. This compound and its preparation are described in U.S. Pat. No. 6,316,456. Analogues of roscovitine are described, for example, in WO 03/002565.

In a preferred embodiment of the invention roscovitine is administered to a mammal or a human, more preferably a human. When performed on an animal model, the invention is preferably performed on a tumour model such as HT29 or A549 xenograft mouse model.

The methods of the present invention where the levels of expression of any of the genes identified herein are monitored will preferably involve monitoring the levels prior to administration of roscovitine and then again preferably 1.5, 2, 3, 4, 5, 8, 12, 24 or 48 hours after administration. In a preferred embodiment, the level is monitored again at least 1.5 hours after administration of roscovitine. In further embodiments, altered protein expression is measured 1 to 10 days after administration.

In one preferred embodiment, the level of a gene detected after administration of roscovitine is preferably lower than that detected prior to administration of roscovitine.

A further aspect of the invention relates to the independent monitoring of roscovitine activity by monitoring altered expression including monitoring the levels of gene expression. In one embodiment, the level of gene expression detected after administration of roscovitine is preferably higher than that detected prior to administration of roscovitine. In another embodiment, the level of gene expression detected after administration of roscovitine is preferably lower than that detected prior to administration of roscovitine.

The methods of the present invention may be further utilised in;

(a) methods of assessing suitable dose levels of roscovitine comprising monitoring the degree and rate of gene expression after administration of roscovitine to a cell, group of cells, animal model or human,

(b) methods of identifying a candidate drug having roscovitine-like activity comprising administering said candidate drug to a cell, group of cells, animal model or human and monitoring the presence or absence of a gene or altered expression of a protein.

Methods such as described in (a) may further comprise correlating the degree and rate of gene expression with the known rate of inhibition of a known gene whose expression is modulated by roscovitine at the same dosage, over the same time period. In one embodiment, phosphorylation status of RB may be compared to the pattern of expression of any one of the genes identified herein. RB as a marker of roscovitine activity is described in WO 02/061386.

In a further aspect, the invention relates to the use of a gene or protein in the monitoring of activity of roscovitine utilising any of the methods described above.

Typically in cell line investigations a CDK2 inhibitory (IC₅₀) dosage of roscovitine is administered and samples extracted over a 24 or 48 hour time period for example at 2, 4, 12, 24 and 48 hours after administration. Protein samples are isolated, loaded and resolved on SDS-PAGE, blotted and probed for the appropriate marker. When conducting investigation in animal models or humans, a suitable proliferating tissue must be identified as being a source of cells that can be extracted from the animal or human for assessment of roscovitine activity. Suitable tissue includes any proliferating tissue. In particular including a tumor biopsy, but it has now been observed that circulating lymphocytes and cells of the buccal mucosa may also be used. Once extracted, these cells can be treated in a manner identical to that described for cell lines. In most cases a pool of markers including a gene as identified herein is identified.

Suitable methods for detecting gene expression in biopsy samples include using FISH or immunohistochemistry techniques using antibodies that recognise the genes identified herein as well as methods for analysing the protein composition of samples.

This embodiment of the invention may be further developed to use the effect of roscovitine on gene expression as a tool in dose titration i.e. by monitoring the degree and rate of gene expression a suitable dose of roscovitine may be determined. Such analysis may further involve correlation of changes of gene expression with the known rate of inhibition of, for example, either CDK2 activity or RB phosphorylation by roscovitine at the same dosage. In this manner, a single measurement of the rate and degree of gene expression may be taken as indicative of further activities of roscovitine.

In an even further embodiment of the invention the altered expression including altered gene expression level by a candidate drug may be taken as an indication of its mode of activity in that it may be classified as roscovitine-like.

Response of a cancer patient to treatment with a particular course of therapy can be highly variable. For example, a patient may be sensitive to treatment with a particular therapy and therefore exhibit reduced tumour burden or improved symptoms. Alternatively, a patient may be resistant to treatment and show no or little improvement in response to a particular therapy. Detecting genes whose expression is modified by a CDKI such as roscovitine may also be useful in methods of identifying markers for the prediction of a response to treatment with a CDKI.

Accordingly, in another aspect there is provided a method for identifying genes whose expression in tumours enables a response to treatment with a CDKI such as roscovitine to be predicted, said method comprising:

a) taking a sample from a patient showing sensitivity to treatment with a CDKI such as roscovitine and detecting expression of at least one of the genes as identified herein;

b) taking a sample from a patient showing resistance to treatment with a CDKI such as roscovitine and detecting expression of at least one of the genes as identified herein; and

c) comparing the patterns of gene expression from a) and b) and therefore identifying those genes which correlate with sensitivity and those which correlate with resistance.

Patterns of gene expression from tumours may then be determined and a particular tumour classified as “sensitive” or “resistant” to treatment according to the expression of those marker genes identified according to the above method.

### BRIEF DESCRIPTION OF THE FIGURES

FIG. 1 shows the results of CYC202 treatment of PBMC, identifying those genes whose expression is significantly down regulated at 1.5 hr along with the corresponding data for expression of those genes at later time points.

FIG. 2 shows the results of CYC202 treatment of PBMC, identifying those genes whose expression is significantly down regulated at 3 hr along with the corresponding data for expression of those genes at later time points.

FIG. 3 shows the identity of the genes corresponding to the probes on the Affymetrix Chips whose expression is down regulated at 1.5 hours (i.e. those probes identified in FIG. 1).

FIG. 4 shows the identity of the genes corresponding to the probes on the Affymetrix Chips whose expression is down regulated at 3 hours (i.e. those probes identified in FIG. 2).

FIG. 5 shows the results of CYC202 treatment of HT29 cells, identifying those genes whose expression is significantly down regulated at 1.5 hr along with the corresponding data for expression of those genes at later time points.

FIG. 6 shows the results of CYC202 treatment of HT29 cells, identifying those genes whose expression is significantly down regulated at 3 hr along with the corresponding data for expression of those genes at later time points.

FIG. 7 shows the identity of the genes corresponding to the probes on the Affymetrix Chips whose expression is down regulated at 1.5 hours (i.e. those probes identified in FIG. 5).

FIG. 8 shows the identity of the genes corresponding to the probes on the Affymetrix Chips whose expression is down regulated at 3 hours (i.e. those probes identified in FIG. 6).

FIG. 9 shows the results of CYC202 treatment of A549 cells, identifying those genes whose expression is significantly down regulated at 2 hours along with the corresponding data for expression of those genes at later time points.

FIG. 10 shows the results of CYC202 treatment of A549 cells, identifying those genes whose expression is significantly down regulated at 4 hours along with the corresponding data for expression of those genes at later time points.

FIG. 11 shows the identity of the genes corresponding to the probes on the Affymetrix Chips whose expression is down regulated at 2 hours (i.e. those probes identified in FIG. 9).

FIG. 12 shows the identity of the genes corresponding to the probes on the Affymetrix Chips whose expression is down regulated at 4 hours (i.e. those probes identified in FIG. 10).

FIG. 13 shows the sequences of the primers used for QPCR analysis.

FIG. 14 shows the sequences of optimised primers used for QPCR analysis.

FIG. 15 shows a comparison of microarray data and Q PCR data for ADM

FIG. 16 shows a comparison of microarray data and Q PCR data for FADD

FIG. 17 shows a comparison of microarray data and Q PCR data for PAI1

FIG. 18 shows a comparison of microarray data and Q PCR data for PLAU

FIG. 19 shows a comparison of microarray data and Q PCR data PNUTS

FIG. 20 shows a comparison of microarray data and Q PCR data for TNFSF14

FIG. 21 shows a comparison of microarray data and Q PCR data for C/EBP alpha

FIG. 22 shows a comparison of microarray data and Q PCR data for 20585

FIG. 23 shows a comparison of microarray data and Q PCR data for FUT4

FIG. 24 shows a comparison of microarray data and Q PCR data for E2F6

FIG. 25 shows a comparison of microarray data and Q PCR data for 18747

FIG. 26 shows a comparison of microarray data and Q PCR data for 22147

FIG. 27 shows a comparison of microarray data and Q PCR data for ZK1

FIG. 28 shows a comparison of microarray data and Q PCR data for KIAA1698

FIG. 29 shows a comparison of microarray data and Q PCR data for CCRL2

FIG. 30 shows a comparison of microarray data and Q PCR data for myc

FIG. 31 shows a comparison of microarray data and Q PCR data for Mcl 1

FIG. 32 shows the effect of CYC202 on the expression of PNUTS in blood prepared using the PAXgene system from several donors (lower panel) and the effect of storage on the CYC202-mediated changes in gene expression in a single donor (upper panel).

FIG. 33 shows pharmacokinetic data for patient 02-2-01 (08) showing the data for the full time-course on Day 1 and a single point prior to dose on Day 5. Plasma concentrations of CYC202 are given in μM.

FIG. 34 is a graph showing the fold decrease in expression of PNUTS, after normalisation with 28S rRNA levels.

FIG. 35 is a graph showing the fold decrease in expression of CEBP, after normalisation with 28S rRNA levels.

FIG. 36 is a graph showing the fold decrease in expression of FUT4, after normalisation with 28S rRNA levels.

FIG. 37 is a graph showing the fold decrease in expression of NM_{—}033410, after normalisation with 28S rRNA levels.

FIG. 38: Top half: Biomarker Wizard plot from analysis of fraction 4 from 16 Phase 1b patients on the SAX chip pH9. Only the mass region between 13.5 and 14.6 kDa is shown here. The Day 1 samples prior to start of treatment are shown (u) and the samples from the last day of treatment are shown (t). Lower half: Representative spectra from two patients showing the appearance of an additional peak following treatment.

FIG. 39 shows 2D gels of patient plasma. Neat plasma samples were applied to IPG strips pH4-7 to resolve proteins in the first dimension by charge and then in the second dimension by SDS-PAGE to separate by size. Molecular weight size markers are on the left of each gel. The spots of interest lie just between the 14 and 17 kDa markers shown on the left of each gel. The lower gels represent an enlarged view of a further two patients, showing that the change is reproducible.

FIG. 40 shows a comparison between the original SELDI-TOF MS profiles and the passive elution sample extracted from 2D gels. Gels were run in duplicate and the 2 spots at approximately 14 kDa in each sample (Day 1 or Day 10) were excised and processed in parallel for passive elution or trypsin digestion. Passive elution allows the extraction of proteins from gel slices and permits their analysis on the SELDI-TOF-MS. This is shown in the top 4 profiles, which are compared to the original SELDI profiles of these samples (shown in the bottom two spectra).

FIG. 41: Top half: Biomarker Wizard plot from analysis of fraction 4 from 16 Phase 1b patients on the SAX chip pH9. Only the mass region between 26.5 kDa and 30.5 kDa is shown here. The Day 1 samples prior to start of treatment are shown (u) and the samples from the last day of treatment are shown (t). The log normalised intensity plots the log of peak intensity, normalising the average intensity to 0, thereby expressing the difference between sample groups regardless of absolute intensity. Lower half: Representative spectra from two patients showing a decrease in the first two peaks, which correspond to the first two biomarkers, and the appearance or increase in the 3^{rd} peak, which corresponds to the third biomarker, following treatment.

FIG. 42 shows 2D gels of patient plasma. Neat plasma samples were applied to IPG strips pH4-7 to resolve proteins in the first dimension by charge and then in the second dimension by SDS-PAGE to separate by size. Molecular weight size markers are on the left of each gel. The spots of interest lie just below the 28 kDa marker.

FIG. 43 shows enlarged views of 2D gels for patients 209 and 116.

FIG. 44 shows 2D gel analysis of patient plasma using a pH3-10 IPG strip. The gel was run in duplicate and the spots at 28 kDa were excised and processed in parallel for passive elution or trypsin digestion.

FIG. 45: Top half: Biomarker Wizard plot from analysis of fraction 6 from 16 Phase 1b patients on the H50 chip. Only the mass region between 5 and 8.5 kDa is shown here. The Day 1 samples prior to start of treatment are shown (u) and the samples from the last day of treatment are shown (t). The log normalised intensity plots the log of peak intensity, normalising the average intensity to 0, thereby expressing the difference between sample groups regardless of absolute intensity. Lower half: Representative spectra from three patients showing the appearance of an additional two peaks following treatment.

FIG. 46: Top half: Biomarker Wizard plot from analysis of neat plasma from 16 Phase 1b patients on the CM10 chip. Only the mass region between 6 and 8 kDa is shown here. The Day 1 samples prior to start of treatment are shown in (u) and the samples from the last day of treatment are shown (t). The log normalised intensity plots the log of peak intensity, normalising the average intensity to 0, thereby expressing the difference between sample groups regardless of absolute intensity. Lower half: Representative spectra from three patients showing the appearance of an additional two peaks following treatment.

### DETAILED DESCRIPTION OF THE INVENTION

The practice of the present invention will employ, unless otherwise indicated, conventional techniques of chemistry, molecular biology, cell biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, IRL Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

By “CDKI” is meant an inhibitor of CDK activity. Roscovitine is just one of a number of compounds known to be inhibitors of CDK activity.

By “roscovitine activity” or “roscovitine-like activity” is meant an activity exhibited by roscovitine. For example, roscovitine-like means capable of inhibiting cell cycle progression in late G1/early S or M phase. Preferably, said inhibition of cell cycle progression is through inhibiting CDKs including CDK1, CDK2, CDK5, CDK7 and CDK9. A study of roscovitine activity is reported in McClue et al. Int. J. Cancer, 2002, 102, 463-468.

The term “marker” or “biomarker” of roscovitine activity is used herein to refer to a gene or protein whose expression in a sample derived from a cell or mammal is altered or modulated, for example, up or down regulated, in response to treatment with roscovitine. Where the biomarker is a protein, modulation or alteration of expression encompasses modulation through different post translational modifications.

Also used herein is the term “biomarker cluster” which means a group of distinct protein forms having a similar mass, when separated by SELDI-TOF MS. Biomarker clusters are described in the Examples section herein.

A sample derived from a treated or untreated cell can be a lysate, extract or nucleic acid sample derived from a group of cells which can be from tissue culture or animal or human. For protein analysis, a sample can be a tissue culture supernatant. A cell can be isolated from an individual (e.g. from a blood, serum or plasma sample) or can be part of a tissue sample such as a biopsy.

By “altered expression” is meant an increase, decrease or otherwise modified level or pattern of expression in a sample derived from a treated cell when compared to an untreated, control sample.

The term “expression” refers to the transcription of a gene's DNA template to produce the corresponding mRNA and translation of this mRNA to produce the corresponding gene product (i.e., a peptide, polypeptide, or protein) as well as the “expression” of a protein in one or more forms that may have been modified post translation.

Post translational modifications are covalent processing events that change the properties of a protein by proteolytic cleavage or by addition of a modifying group to one or more amino acids. Common post translational modifications include phosphorylation, acetylation, methylation, acylation, glycosylation, GPI anchor, ubiquitination and so forth. A review of such modifications and methods for detection may be found in Mann et al. Nature Biotechnology March 2003, Vol. 21, pages 255-261.

By “polynucleotide” or “polypeptide” is meant the DNA and protein sequences disclosed herein whose expression is modified in response to roscovitine. The terms also include close variants of those sequences, where the variant possesses the same biological activity as the reference sequence. Such variant sequences include “alleles” (variant sequences found at the same genetic locus in the same or closely-related species), “homologs” (a gene related to a second gene by descent from a common ancestral DNA sequence, and separated by either speciation (“ortholog”) or genetic duplication (“paralog”)), so long as such variants retain the same biological activity as the reference sequence(s) disclosed herein.

The invention is also intended to include detection of genes having silent polymorphisms and conservative substitutions in the polynucleotides and polypeptides disclosed herein, so long as such variants retain the same biological activity as the reference sequence(s) as disclosed herein.

Measuring Altered Expression of Gene and Protein Markers of CDKI Activity

Levels of gene and protein expression may be determined using a number of different techniques.

a) At the RNA Level

Gene expression can be detected at the RNA level. RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland). Typical assay formats utilising ribonucleic acid hybridisation include nuclear run-on assays, RT-PCR, RNase protection assays (Melton et al., Nuc. Acids Res. 12:7035), Northern blotting and In Situ hybridization. Gene expression can also be detected by microarray analysis as described below.

For Northern blotting, RNA samples are first separated by size via electrophoresis in an agarose gel under denaturing conditions. The RNA is then transferred to a membrane, crosslinked and hybridized with a labeled probe. Nonisotopic or high specific activity radiolabeled probes can be used including random-primed, nick-translated, or PCR-generated DNA probes, in vitro transcribed RNA probes, and oligonucleotides. Additionally, sequences with only partial homology (e.g., cDNA from a different species or genomic DNA fragments that might contain an exon) may be used as probes.

Nuclease Protection Assays (including both ribonuclease protection assays and S1 nuclease assays) provide an extremely sensitive method for the detection and quantitation of specific mRNAs. The basis of the NPA is solution hybridization of an antisense probe (radiolabeled or nonisotopic) to an RNA sample. After hybridization, single-stranded, unhybridized probe and RNA are degraded by nucleases. The remaining protected fragments are separated on an acrylamide gel. NPAs allow the simultaneous detection of several RNA species.

In situ hybridization (ISH) is a powerful and versatile tool for the localization of specific mRNAs in cells or tissues. Hybridization of the probe takes place within the cell or tissue. Since cellular structure is maintained throughout the procedure, ISH provides information about the location of mRNA within the tissue sample.

The procedure begins by fixing samples in neutral-buffered formalin, and embedding the tissue in paraffin. The samples are then sliced into thin sections and mounted onto microscope slides. (Alternatively, tissue can be sectioned frozen and post-fixed in paraformaldehyde.) After a series of washes to dewax and rehydrate the sections, a Proteinase K digestion is performed to increase probe accessibility, and a labeled probe is then hybridized to the sample sections. Radiolabeled probes are visualized with liquid film dried onto the slides, while nonisotopically labeled probes are conveniently detected with colorimetric or fluorescent reagents. This latter method of detection is the basis for Fluorescent In Situ Hybridisation (FISH).

Methods for detection which can be employed include radioactive labels, enzyme labels, chemiluminescent labels, fluorescent labels and other suitable labels.

Typically, RT-PCR is used to amplify RNA targets. In this process, the reverse transcriptase enzyme is used to convert RNA to complementary DNA (cDNA) which can then be amplified to facilitate detection. Relative quantitative RT-PCR involves amplifying an internal control simultaneously with the gene of interest. The internal control is used to normalize the samples. Once normalized, direct comparisons of relative abundance of a specific mRNA can be made across the samples. Commonly used internal controls include, for example, GAPDH, HPRT, actin and cyclophilin.

Many DNA amplification methods are known, most of which rely on an enzymatic chain reaction (such as a polymerase chain reaction, a ligase chain reaction, or a self-sustained sequence replication) or from the replication of all or part of the vector into which it has been cloned.

Many target and signal amplification (TAS) methods have been described in the literature, for example, general reviews of these methods in Landegren, U. et al., Science 242:229-237 (1988) and Lewis, R., Genetic Engineering News 10:1, 54-55 (1990).

PCR is a nucleic acid amplification method described inter alia in U.S. Pat. Nos. 4,683,195 and 4,683,202. PCR can be used to amplify any known nucleic acid in a diagnostic context (Mok et al., 1994, Gynaecologic Oncology 52:247-252). Self-sustained sequence replication (3SR) is a variation of TAS, which involves the isothermal amplification of a nucleic acid template via sequential rounds of reverse transcriptase (RT), polymerase and nuclease activities that are mediated by an enzyme cocktail and appropriate oligonucleotide primers (Guatelli et al., 1990, Proc. Natl. Acad. Sci. USA 87:1874). Ligation amplification reaction or ligation amplification system uses DNA ligase and four oligonucleotides, two per target strand. This technique is described by Wu, D. Y. and Wallace, R. B., 1989, Genomics 4:560. In the Qβ Replicase technique, RNA replicase for the bacteriophage Qβ, which replicates single-stranded RNA, is used to amplify the target DNA, as described by Lizardi et al., 1988, Bio/Technology 6:1197.

Quantitative PCR (Q-PCR) is a technique which allows relative amounts of transcripts within a sample to be determined. A suitable method for performing QPCR is described herein.

Alternative amplification technology can be exploited in the present invention. For example, rolling circle amplification (Lizardi et al., 1998, Nat Genet 19:225) is an amplification technology available commercially (RCAT™) which is driven by DNA polymerase and can replicate circular oligonucleotide probes with either linear or geometric kinetics under isothermal conditions. A further technique, strand displacement amplification (SDA; Walker et al., 1992, Proc. Natl. Acad. Sci. USA 80:392) begins with a specifically defined sequence unique to a specific target.

Suitable probes for detecting the markers of roscovitine activity identified herein may conveniently be packaged in the form of a test kit in a suitable container. In such kits the probe may be bound to a solid support where the assay format for which the kit is designed requires such binding. The kit may also contain suitable reagents for treating the sample to be probed, hybridising the probe to nucleic acid in the sample, control reagents, instructions, and the like. Suitable kits may comprise, for example, primers for a QPCR reaction or labelled probes for performing FISH.

b) At the Polypeptide Level

Altered gene or protein expression may also be detected by measuring the polypeptides encoded by the gene markers of roscovitine activity. This may be achieved by using molecules which bind to the polypeptides encoded by any one of the genes identified herein as a marker of roscovitine activity. Suitable molecules/agents which bind either directly or indirectly to the polypeptides in order to detect the presence of the protein include naturally occurring molecules such as peptides and proteins, for example antibodies, or they may be synthetic molecules.

Methods for production of antibodies are known by those skilled in the art. If polyclonal antibodies are desired, a selected mammal (e.g., mouse, rabbit, goat, horse, etc.) is immunised with an immunogenic polypeptide bearing an epitope(s) from a polypeptide. Serum from the immunised animal is collected and treated according to known procedures. If serum containing polyclonal antibodies to an epitope from a polypeptide contains antibodies to other antigens, the polyclonal antibodies can be purified by immunoaffinity chromatography. Techniques for producing and processing polyclonal antisera are known in the art. In order to generate a larger immunogenic response, polypeptides or fragments thereof may be haptenised to another polypeptide for use as immunogens in animals or humans.

Monoclonal antibodies directed against epitopes in polypeptides can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against epitopes in the polypeptides of the invention can be screened for various properties; i.e., for isotype and epitope affinity.

An alternative technique involves screening phage display libraries where, for example the phage express scFv fragments on the surface of their coat with a large variety of complementarity determining regions (CDRs). This technique is well known in the art.

For the purposes of this invention, the term “antibody”, unless specified to the contrary, includes fragments of whole antibodies which retain their binding activity for a target antigen. Such fragments include Fv, F(ab′) and F(ab′)₂ fragments, as well as single chain antibodies (scFv). Furthermore, the antibodies and fragments thereof may be humanised antibodies, for example as described in EP-A-239400.

Standard laboratory techniques such as immunoblotting as described above can be used to detect altered levels of markers of roscovitine activity, as compared with untreated cells in the same cell population.

Gene expression may also be determined by detecting changes in post-translational processing of polypeptides or post-transcriptional modification of nucleic acids. For example, differential phosphorylation of polypeptides, the cleavage of polypeptides or alternative splicing of RNA, and the like may be measured. Levels of expression of gene products such as polypeptides, as well as their post-translational modification, may be detected using proprietary protein assays or techniques such as 2D polyacrylamide gel electrophoresis.

Antibodies may be used in detecting markers of roscovitine activity identified herein in biological samples by a method which comprises: (a) providing an antibody of the invention; (b) incubating a biological sample with said antibody under conditions which allow for the formation of an antibody-antigen complex; and (c) determining whether antibody-antigen complex comprising said antibody is formed.

Suitable samples include extracts of tissues such as brain, breast, ovary, lung, colon, pancreas, testes, liver, muscle and bone tissues or from neoplastic growths derived from such tissues. Other suitable examples include blood or urine samples.

Antibodies that specifically bind to protein markers of roscovitine activity can be used in diagnostic methods and kits that are well known to those of ordinary skill in the art to detect or quantify the markers of roscovitine activity proteins in a body fluid or tissue. Results from these tests can be used to diagnose or predict the occurrence or recurrence of a cancer and other cell cycle progression-mediated diseases or to assess the effectiveness of drug dosage and treatment.

Antibodies can be assayed for immunospecific binding by any method known in the art. The immunoassays which can be used include but are not limited to competitive and non-competitive assay systems using techniques such as western blots, immunohistochemistry, radioimmunoassays, ELISA, sandwich immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays. Such assays are routine in the art (see, for example, Ausubel et al., eds, 1994, Current Protocols in Molecular Biology, Vol. 1, John Wiley & Sons, Inc., New York, which is incorporated by reference herein in its entirety).

Antibodies for use in the invention may be bound to a solid support and/or packaged into kits in a suitable container along with suitable reagents, controls, instructions and the like.

Other methods include 2D-PAGE although this is not suitable for large-scale screening. Newer techniques include matrix-assisted laser desorption ionization time of flight mass spectrometry (MALDI-TOF MS). In MALDI-TOF analysis, proteins in a complex mixture are affixed to a solid metallic matrix, desorbed with a pulsed laser beam to generate gas-phase ions that traverse a field-free flight tube, and are then separated according to their mass-dependent velocities. Individual proteins and peptides can be identified through the use of informatics tools to search protein and peptide sequence databases. Surface-enhanced laser desorption/ionisation time of flight MS (SELDI-TOF MS) is an affinity-based MS method in which proteins are selectively adsorbed to a chemically modified solid surface, impurities are removed by washing, an energy-absorbing matrix is applied, and the proteins are identified by laser desorption mass analysis.

In order to identify protein biomarkers, SELDI-TOF-MS can be used for the detection of the appearance/loss of either intact proteins or fragments of specific proteins. In addition SELDI-TOF-MS can also be used for detection of post translational modifications of proteins due to the difference in mass caused by the addition/removal of chemical groups. Thus phosphorylation of a single residue will cause a mass shift of 80 Da due to the phosphate group. A data base of molecular weights that can be attributed to post-translational modifications is freely accessible on the internet (http://www.abrf.org/index.cfin/dm.home?avgmass=all). Moreover specific polypeptides can be captured by affinity-based approaches using SELDI-TOF-MS by employing antibodies that specifically recognise a post-translationally modified form of the protein, or that can recognise all forms of the protein equally well.

Arrays

Array technology and the various techniques and applications associated with it is described generally in numerous textbooks and documents. These include Lemieux et al., 1998, Molecular Breeding 4:277-289; Schena and Davis. Parallel Analysis with Biological Chips. in PCR Methods Manual (eds. M. Innis, D. Gelfand, J. Sninsky); Schena and Davis, 1999, Genes, Genomes and Chips. In DNA Microarrays: A Practical Approach (ed. M. Schena), Oxford University Press, Oxford, UK, 1999); The Chipping Forecast (Nature Genetics special issue; January 1999 Supplement); Mark Schena (Ed.), Microarray Biochip Technology, (Eaton Publishing Company); Cortes, 2000, The Scientist 14(17):25; Gwynne and Page, Microarray analysis: the next revolution in molecular biology, Science, 1999, Aug. 6; Eakins and Chu, 1999, Trends in Biotechnology, 17:217-218, and also at various world wide web sites.

Array technology overcomes the disadvantages with traditional methods in molecular biology, which generally work on a “one gene in one experiment” basis, resulting in low throughput and the inability to appreciate the “whole picture” of gene function. Currently, the major applications for array technology include the identification of sequence (gene/gene mutation) and the determination of expression level (abundance) of genes. Gene expression profiling may make use of array technology, optionally in combination with proteomics techniques (Celis et al., 2000, FEBS Lett, 480(1):2-16; Lockhart and Winzeler, 2000, Nature 405(6788):827-836; Khan et al., 1999, 20(2):223-9). Other applications of array technology are also known in the art; for example, gene discovery, cancer research (Marx, 2000, Science 289: 1670-1672; Scherf et alet al., 2000, Nat Genet 24(3):236-44; Ross et al., 2000, Nat Genet 2000, 24(3):227-35), SNP analysis (Wang et al., 1998, Science 280(5366):1077-82), drug discovery, pharmacogenomics, disease diagnosis (for example, utilising microfluidics devices: Chemical & Engineering News, Feb. 22, 1999, 77(8):27-36), toxicology (Rockett and Dix (2000), Xenobiotica 30(2):155-77; Afshari et al., 1999, Cancer Res 59(19):4759-60) and toxicogenomics (a hybrid of functional genomics and molecular toxicology). The goal of toxicogenomics is to find correlations between toxic responses to toxicants and changes in the genetic profiles of the objects exposed to such toxicants (Nuwaysir et al., 1999, Molecular Carcinogenesis 24:153-159).

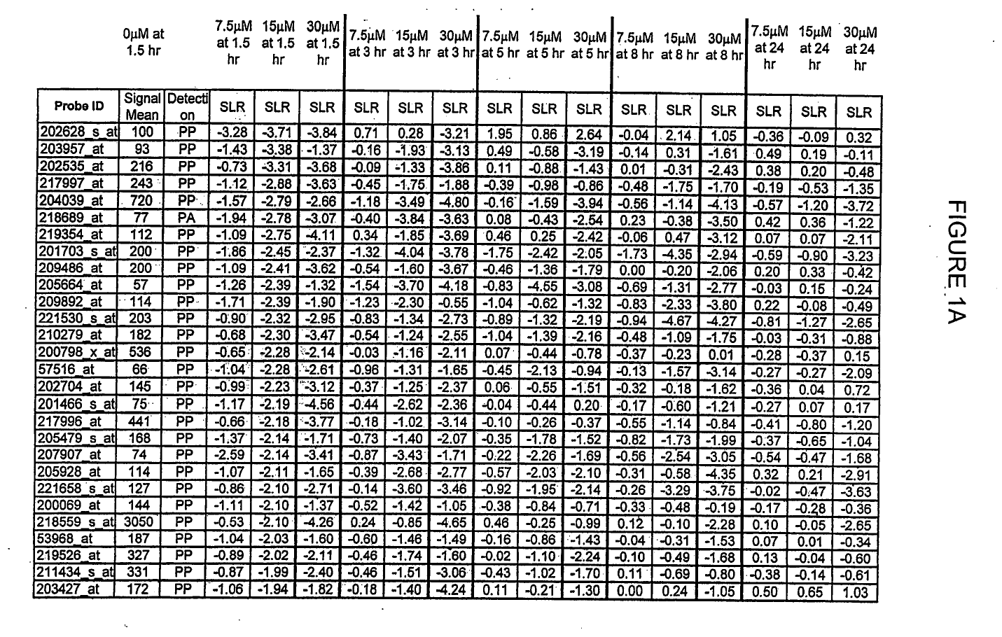

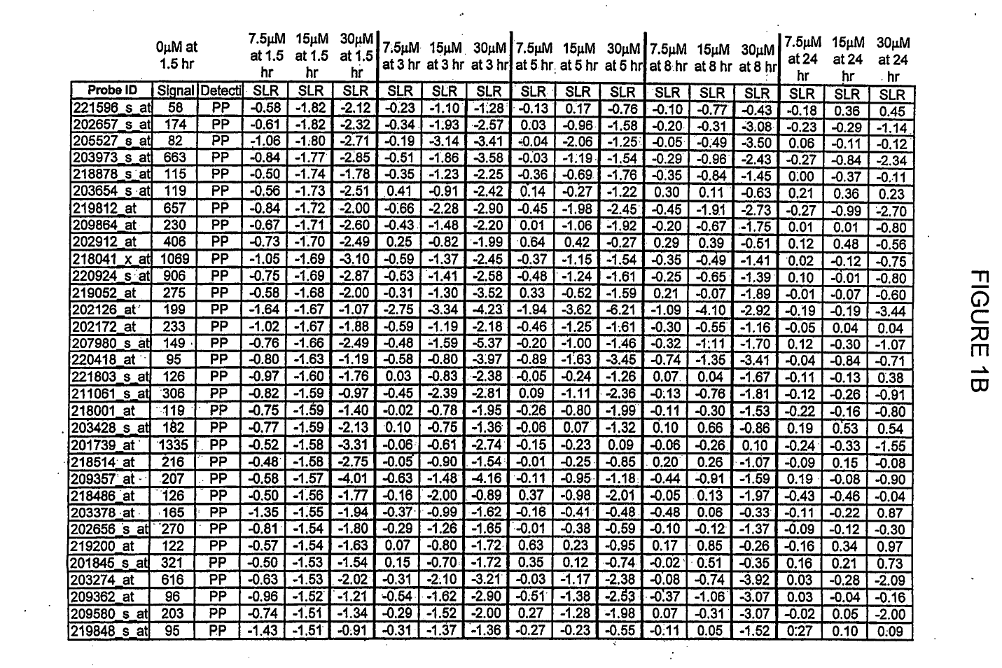

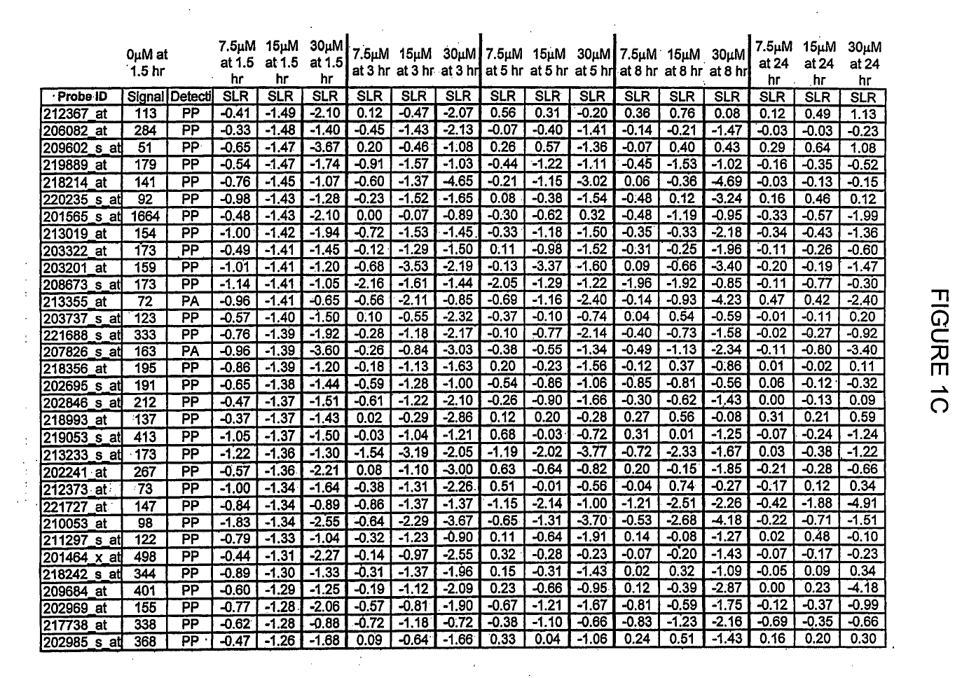

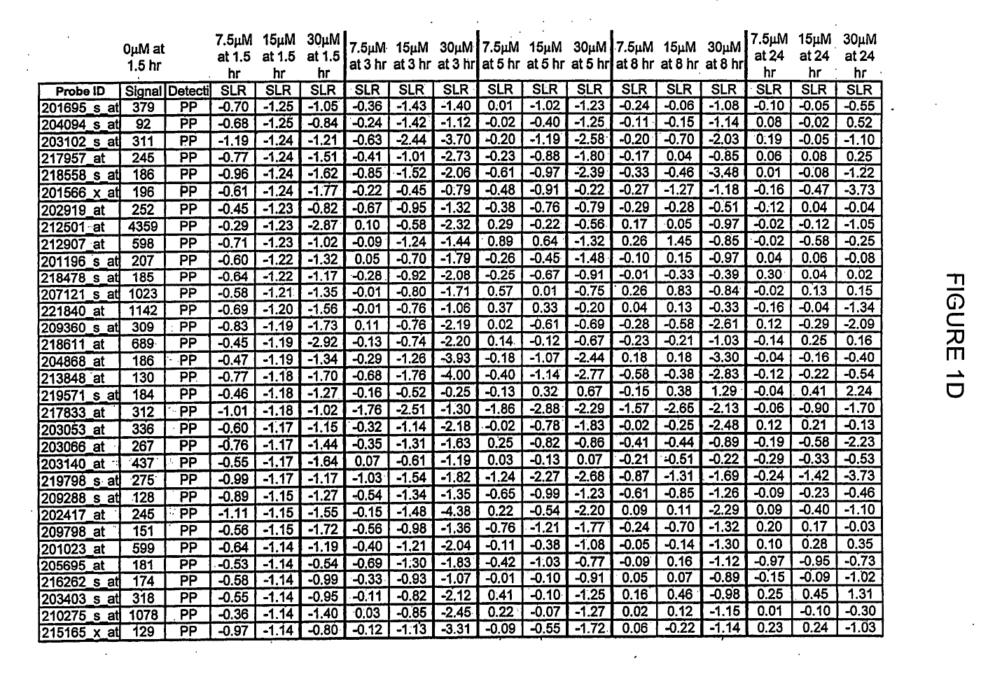

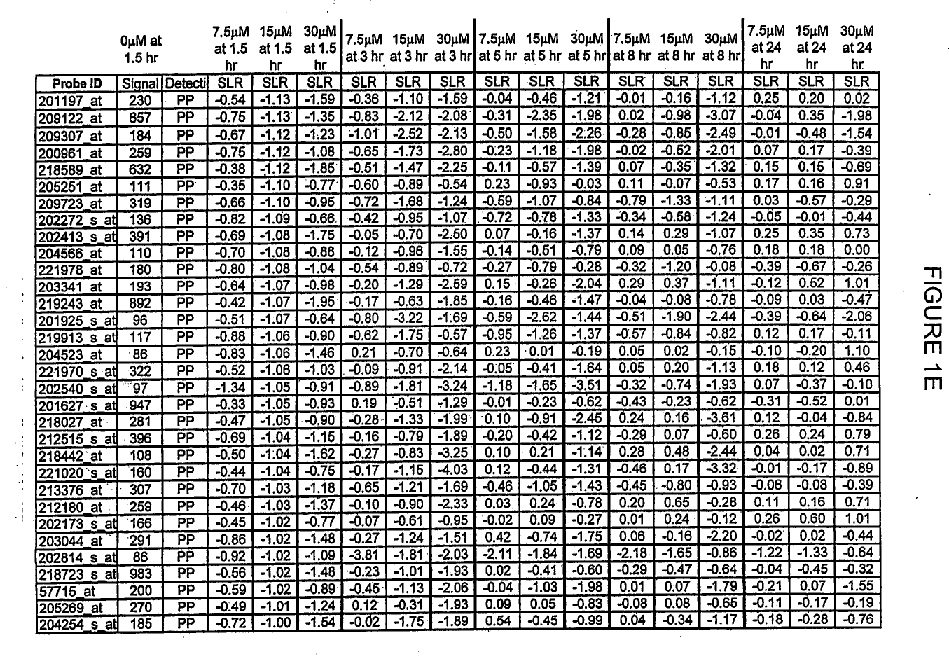

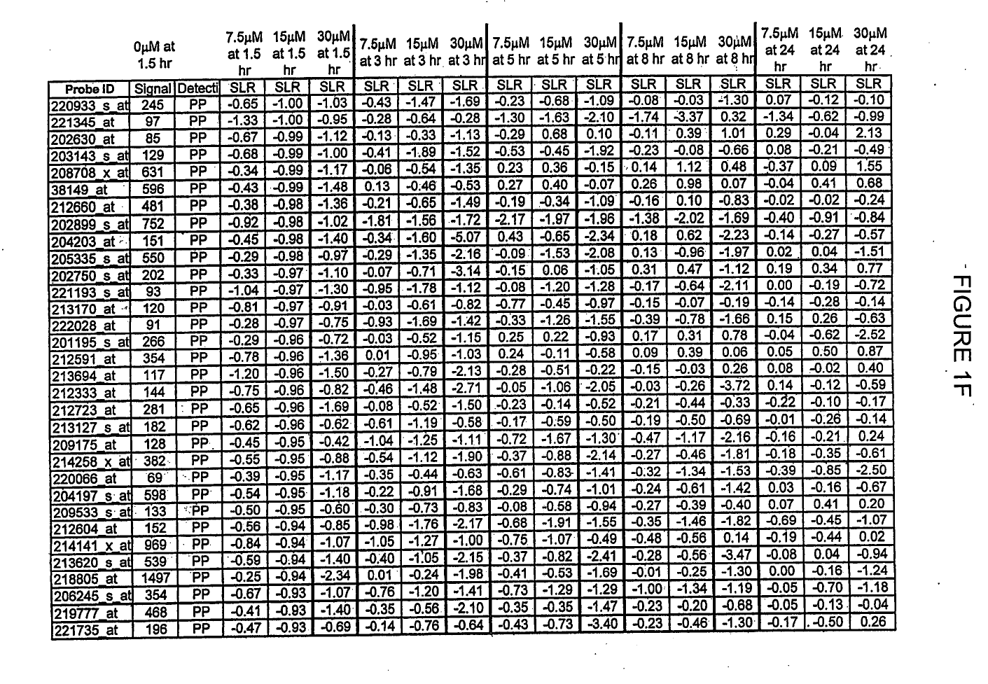

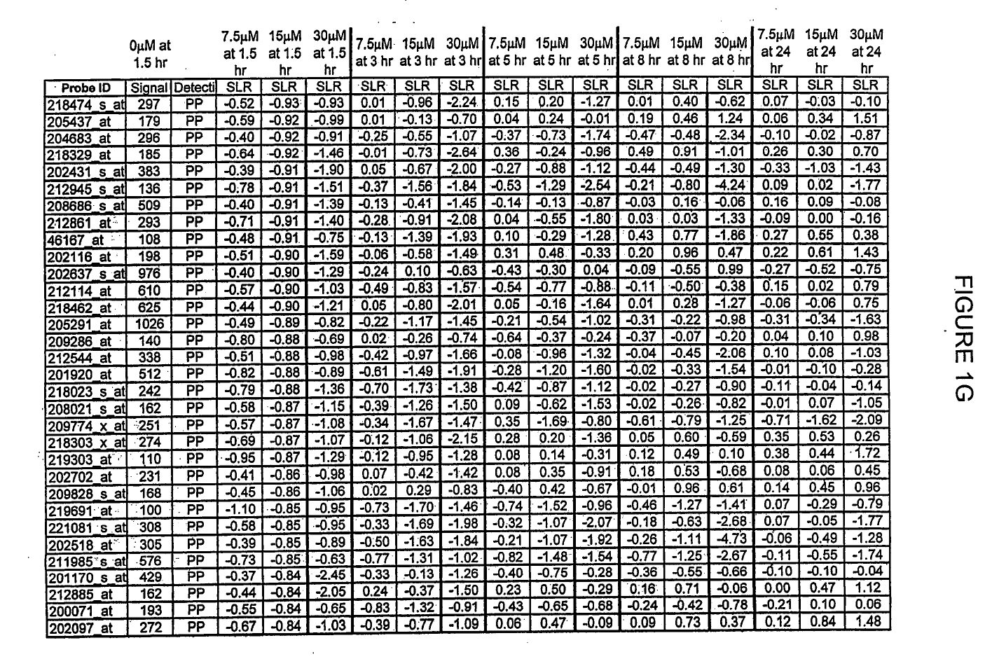

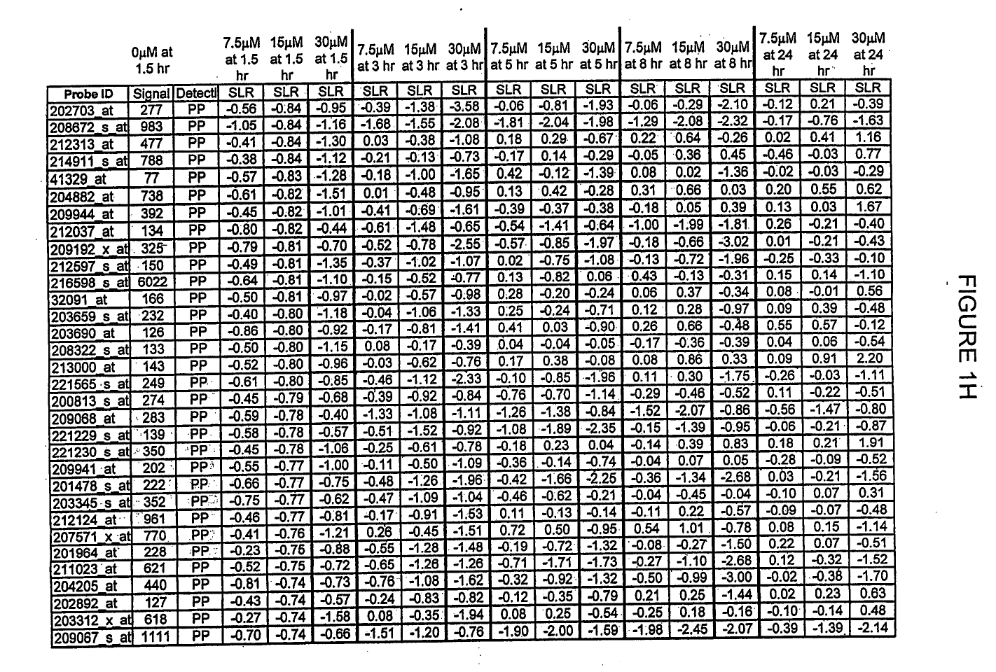

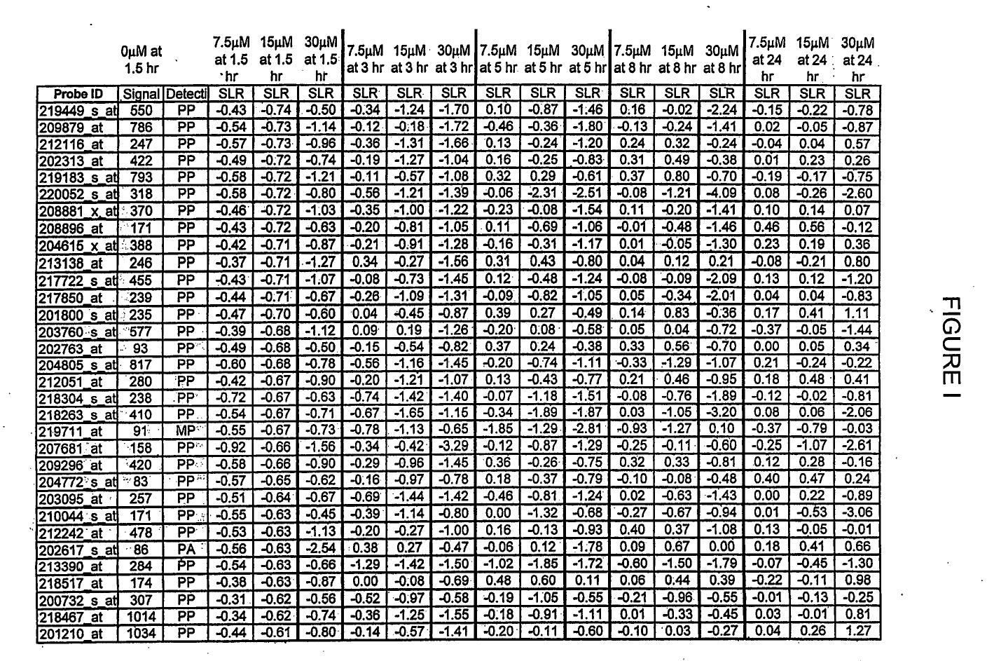

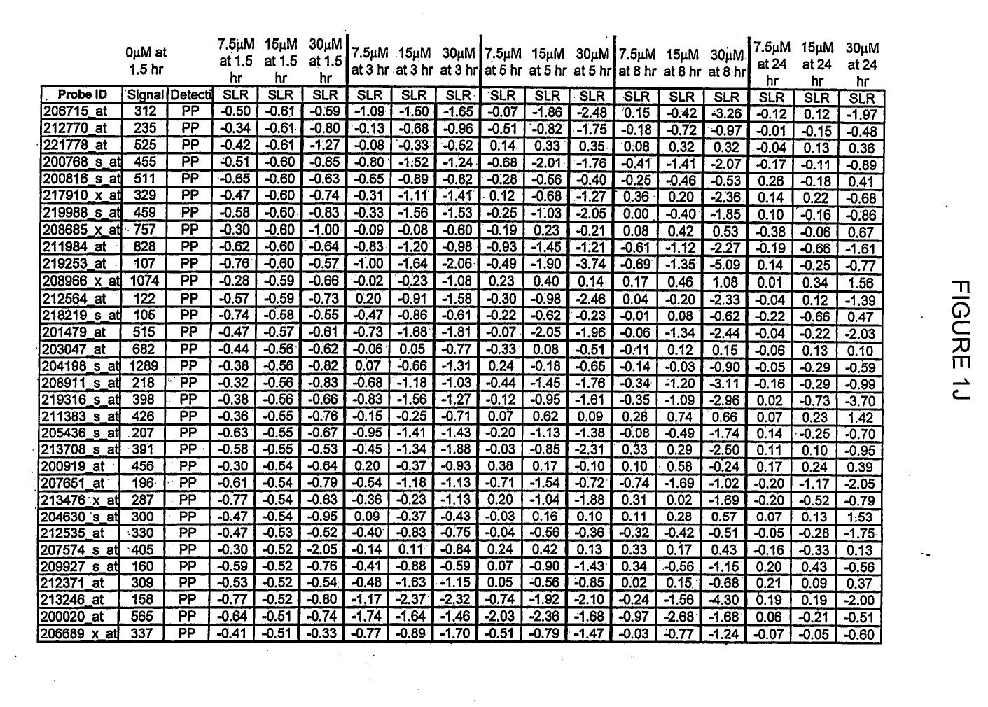

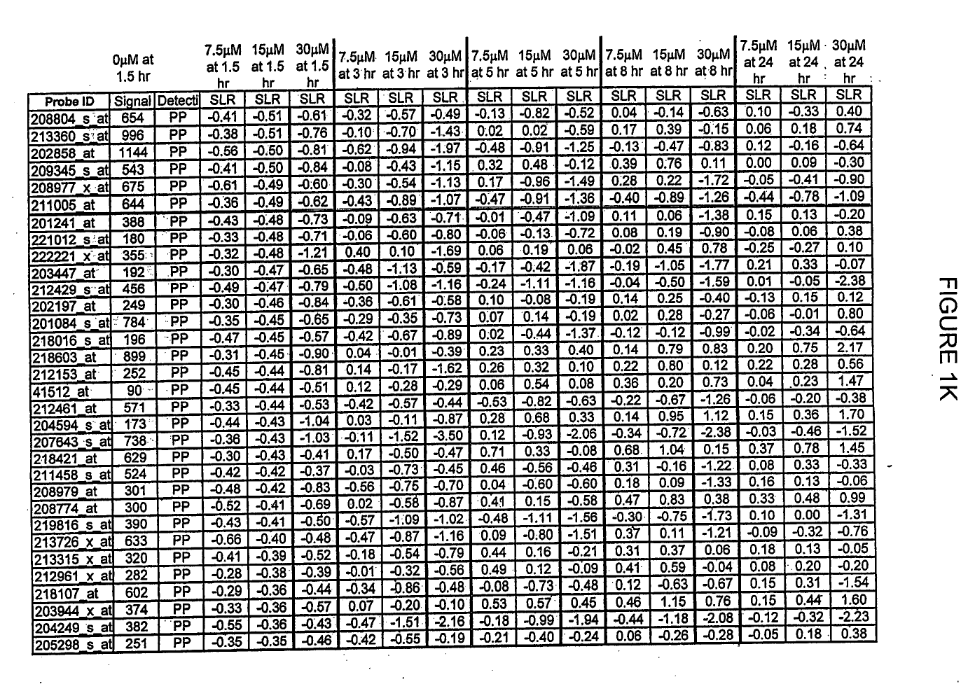

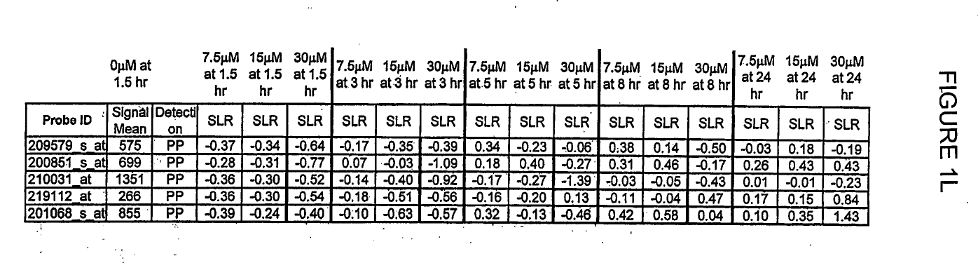

In the context of the present invention, array technology can be used, for example, in the analysis of the expression of one or more of the protein markers of roscovitine activity identified herein. In one embodiment, array technology may be used to assay the effect of a candidate compound on a number of the markers of roscovitine activity identified herein simultaneously. Accordingly, another aspect of the present invention is to provide microarrays that include at least one, at least two or at least several of the nucleic acids identified in any of FIGS. 1 to 12, or fragments thereof, or protein or antibody arrays.

In general, any library or group of samples may be arranged in an orderly manner into an array, by spatially separating the members of the library or group. Examples of suitable libraries for arraying include nucleic acid libraries (including DNA, cDNA, oligonucleotide, etc. libraries), peptide, polypeptide and protein libraries, as well as libraries comprising any molecules, such as ligand libraries, among others. Accordingly, where reference is made to a “library” in this document, unless the context dictates otherwise, such reference should be taken to include reference to a library in the form of an array. In the context of the present invention, a “library” may include a sample of markers of roscovitine activity as identified herein.

The samples (e.g., members of a library) are generally fixed or immobilised onto a solid phase, preferably a solid substrate, to limit diffusion and admixing of the samples. In a preferred embodiment, libraries of DNA binding ligands may be prepared. In particular, the libraries may be immobilised to a substantially planar solid phase, including membranes and non-porous substrates such as plastic and glass. Furthermore, the samples are preferably arranged in such a way that indexing (i.e., reference or access to a particular sample) is facilitated. Typically the samples are applied as spots in a grid formation. Common assay systems may be adapted for this purpose. For example, an array may be immobilised on the surface of a microplate, either with multiple samples in a well, or with a single sample in each well. Furthermore, the solid substrate may be a membrane, such as a nitrocellulose or nylon membrane (for example, membranes used in blotting experiments). Alternative substrates include glass, or silica based substrates. Thus, the samples are immobilised by any suitable method known in the art, for example, by charge interactions, or by chemical coupling to the walls or bottom of the wells, or the surface of the membrane. Other means of arranging and fixing may be used, for example, pipetting, drop-touch, piezoelectric means, ink-jet and bubblejet technology, electrostatic application, etc. In the case of silicon-based chips, photolithography may be utilised to arrange and fix the samples on the chip.

The samples may be arranged by being “spotted” onto the solid substrate; this may be done by hand or by making use of robotics to deposit the sample. In general, arrays may be described as macroarrays or microarrays, the difference being the size of the sample spots. Macroarrays typically contain sample spot sizes of about 300 microns or larger and may be easily imaged by existing gel and blot scanners. The sample spot sizes in microarrays are typically less than 200 microns in diameter and these arrays usually contain thousands of spots. Thus, microarrays may require specialized robotics and imaging equipment, which may need to be custom made. Instrumentation is described generally in a review by Cortese, 2000, The Scientist 14(11):26.

Techniques for producing immobilised libraries of DNA molecules have been described in the art. Generally, most prior art methods described how to synthesise single-stranded nucleic acid molecule libraries, using for example masking techniques to build up various permutations of sequences at the various discrete positions on the solid substrate. U.S. Pat. No. 5,837,832, the contents of which are incorporated herein by reference, describes an improved method for producing DNA arrays immobilised to silicon substrates based on very large scale integration technology. In particular, U.S. Pat. No. 5,837,832 describes a strategy called “tiling” to synthesize specific sets of probes at spatially-defined locations on a substrate which may be used to produced the immobilised DNA libraries of the present invention. U.S. Pat. No. 5,837,832 also provides references for earlier techniques that may also be used.

Arrays of peptides (or peptidomimetics) may also be synthesised on a surface in a manner that places each distinct library member (e.g., unique peptide sequence) at a discrete, predefined location in the array. The identity of each library member is determined by its spatial location in the array. The locations in the array where binding interactions between a predetermined molecule (e.g., a target or probe) and reactive library members occur is determined, thereby identifying the sequences of the reactive library members on the basis of spatial location. These methods are described in U.S. Pat. No. 5,143,854; WO 90/15070 and WO 92/10092; Fodor et al., 1991, Science 251:767; Dower and Fodor, 1991, Ann. Rep. Med. Chem. 26:271.

To aid detection, targets and probes may be labelled with any readily detecFigure reporter, for example, a fluorescent, bioluminescent, phosphorescent, radioactive, etc reporter. Such reporters, their detection, coupling to targets/probes, etc are discussed elsewhere in this document. Labelling of probes and targets is also disclosed in Shalon et al., 1996, Genome Res 6(7):639-45.

Specific examples of DNA arrays include the following:

Format I: probe cDNA (˜500-˜5,000 bases long) is immobilized to a solid surface such as glass using robot spotting and exposed to a set of targets either separately or in a mixture. This method is widely considered as having been developed at Stanford University (Ekins and Chu, 1999, Trends in Biotechnology, 17:217-218).

Format II: an array of oligonucleotide (˜20-˜25-mer oligos) or peptide nucleic acid (PNA) probes is synthesized either in situ (on-chip) or by conventional synthesis followed by on-chip immobilization. The array is exposed to labeled sample DNA, hybridized, and the identity/abundance of complementary sequences are determined. Such a DNA chip is sold by Affymetrix, Inc., under the GeneChip® trademark.

Examples of some commercially available microarray formats are set out, for example, in Marshall and Hodgson, 1998, Nature Biotechnology 16(1):27-31.

Data analysis is also an important part of an experiment involving arrays. The raw data from a microarray experiment typically are images, which need to be transformed into gene expression matrices—Figureswhere rows represent for example genes, columns represent for example various samples such as tissues or experimental conditions, and numbers in each cell for example characterize the expression level of the particular gene in the particular sample. These matrices have to be analyzed further, if any knowledge about the underlying biological processes is to be extracted. Methods of data analysis (including supervised and unsupervised data analysis as well as bioinformatics approaches) are disclosed in Brazma and Vilo J, 2000, FEBS Lett 480(1):17-24.

As disclosed above, proteins, polypeptides, etc may also be immobilised in arrays. For example, antibodies have been used in microarray analysis of the proteome using protein chips (Borrebaeck C A, 2000, Immunol Today 21(8):379-82). Polypeptide arrays are reviewed in, for example, MacBeath and Schreiber, 2000, Science, 289(5485):1760-1763.

Diagnostics and Prognostics

The invention also includes use of the markers of roscovitine activity, antibodies to those proteins, and compositions comprising those proteins and/or their antibodies in diagnosis or prognosis of diseases characterized by proliferative activity, particularly in individuals being treated with roscovitine. As used herein, the term “prognostic method” means a method that enables a prediction regarding the progression of a disease of a human or animal diagnosed with the disease, in particular, cancer. In particular, cancers of interest with respect to roscovitine treatment include breast, lung, gastric, head and neck, colorectal, renal, pancreatic, uterine, hepatic, bladder, endometrial and prostate cancers and leukemias.

The term “diagnostic method” as used herein means a method that enables a determination of the presence or type of cancer in or on a human or animal. Suitably the marker allows success of roscovitine treatment to be assessed. As discussed above, suitable diagnostics include probes directed to any of the genes as identified herein such as, for example, QPCR primers, FISH probes and so forth.

The present invention will now be described with reference to the following examples.

### EXAMPLES

### Example 1

### Identification of Genes Expressed in CYC202 Treated Cells

Methods

Cell Culture

Peripheral Blood Mononuclear Cells (PBMC) were purified by centrifugation in Vacutainer CPT tubes (Becton Dickinson, N.J., USA) according to the manufacturer's instructions. The cells were seeded at a density of approximately 2×10⁷ cells in 40 ml RPMI medium containing 10% foetal calf serum and penicillin-streptomycin.

HT29 and A549 cells, which are derived from human colon and lung tumours respectively, were seeded at approximately 20% confluency, which was equivalent to 1.5×10⁶ cells per 10 cm plate in DMEM containing 10% FCS, so that they were actively proliferating upon treatment with the compound the following day.

All cell cultures were incubated overnight in a 37° C. incubator in the presence of 5% CO₂, prior to treatment with CYC202 or the vehicle control, DMSO. PBMC and HT29 cells were treated with CYC202 at 7.5, 15 or 30 μM and samples taken at 1.5, 3, 5, 8 and 24 hours. A549 cells were treated with CYC202 at 15, 45 and 75 μM and samples were taken at 2, 4, 8 and 24 hours.

RNA Extraction

RNA was extracted from the A549 cells used in the microarray experiment using TRIZOL reagent (Invitrogen) according to the manufacturer's instructions. For all other cell lines, total RNA was extracted from the cultures with the RNeasy Midi Kit (Qiagen, Hilden, Germany) according to the manufacturer's instructions. Cells were lysed in Buffer RLT, then passed through Qiashredder columns prior to RNA extraction. Samples were treated with RNase-free DNase (Qiagen) while bound to the columns. RNA was eluted in RNase-free water and quantified using RiboGreen (Molecular Probes, Leiden, The Netherlands). Aliquots of the samples were run on agarose gels to check the integrity and quality of the RNA.

Synthesis of Double-Stranded cDNA

10 μg total RNA was used as starting material for the cDNA preparation. The first and second strand cDNA synthesis was performed using the SuperScript II System (Invitrogen, Carlsbad, Calif.) according to the manufacturer's instructions except using an oligo-dT primer containing a T7 RNA polymerase promoter site. Labelled cRNA was prepared using the BioArray High Yield RNA Transcript Labelling Kit (Enzo). Biotin labelled CTP and UTP (Enzo) were used in the reaction together with unlabeled NTP's. Following the IVT reaction, the unincorporated nucleotides were removed using RNeasy columns (Qiagen, Hilden, Germany).

Microarray Hybridisation and Scanning

The cRNA was fragmented by metal-induced hydrolysis according to Affymetrix Technical Manual, resulting in fragments of between 35 and 200 bases. 15 μg of cRNA was fragmented at 94° C. for 35 min in a fragmentation buffer containing 40 mM Tris-acetate pH 8.1, 100 mM KOAc, 30 mM MgOAc. Prior to hybridisation, the fragmented cRNA in a 6×SSPE-T hybridisation buffer (1 M NaCl, 10 mM Tris pH 7.6, 0.005% Triton), was heated to 95° C. for 5 min and subsequently to 45° C. for 5 min before loading onto the Affymetrix probe array cartridge.

RNA extracted from A549 cells was used with the Hu Gene FL Cartridge while RNA from HT-29 cells and PBMC was loaded onto Hu Gene U133A Cartridges. The probe array was then incubated for 16 h at 45° C. at constant rotation (60 rpm). The washing and staining procedure was performed in the Affymetrix Fluidics Station. The probe array was exposed to 10 washes in 6×SSPE-T at 25° C. followed by 4 washes in 0.5×SSPE-T at 50° C. The biotinylated cRNA was detected with an antibody amplification step using normal goat IgG as blocking reagent, final concentration 0.1 mg/ml (Sigma) and biotinylated anti-streptavidin antibody (goat), final concentration 3 mg/ml (Vector Laboratories). This was followed by a staining step with a streptavidin-phycoerythrin conjugate, final concentration 2 mg/ml (Molecular Probes, Eugene, Oreg.) in 6×SSPE-T for 30 min at 25° C. and 10 washes in 6×SSPE-T at 25° C. The probe arrays were scanned at 560 nm using a confocal laser-scanning microscope (Hewlett Packard GeneArray Scanner G2500A). The readings from the quantitative scanning were analysed by the Affymetrix Gene Expression Analysis Software.

Analysis of Microarray Data

Data analysis was performed using the Affymetrix Software Packages; MAS ver. 5.0, MicroDB ver. 3.0, and DMT ver. 3.0. All raw analyses were Global Scaled to 150 units, and subsequently compared by Pairwise Comparison analysis.

Selection of Genes of Interest

Data from the Affymetrix Chips were analysed at individual time points following CYC202 treatment. The genes identified as markers of CYC202 treatment are those that were present in the RNA isolated from cells treated with DMSO but whose expression was determined to be down regulated in the RNA isolated from cells treated with all concentrations of CYC202.

Expression data obtained in microarrays using samples derived from PBMC treated with CYC202 at 7.5, 15 and 30 micromolar for 1.5, 3, 5, 8 and 24 hrs is given in the following Figures.

FIG. 1 shows the results of CYC202 treatment of PBMC, identifying those genes whose expression is significantly down regulated at 1.5 hr along with the corresponding data for expression of those genes at later time points.

FIG. 2 shows the results of CYC202 treatment of PBMC, identifying those genes whose expression is significantly down regulated at 3 hr along with the corresponding data for expression of those genes at later time points.

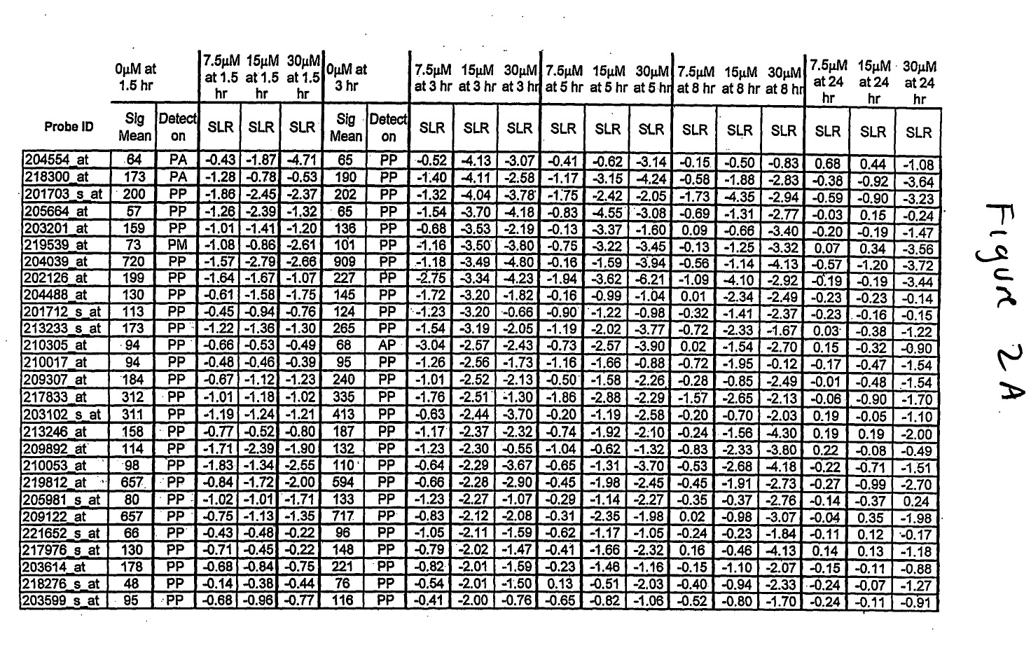

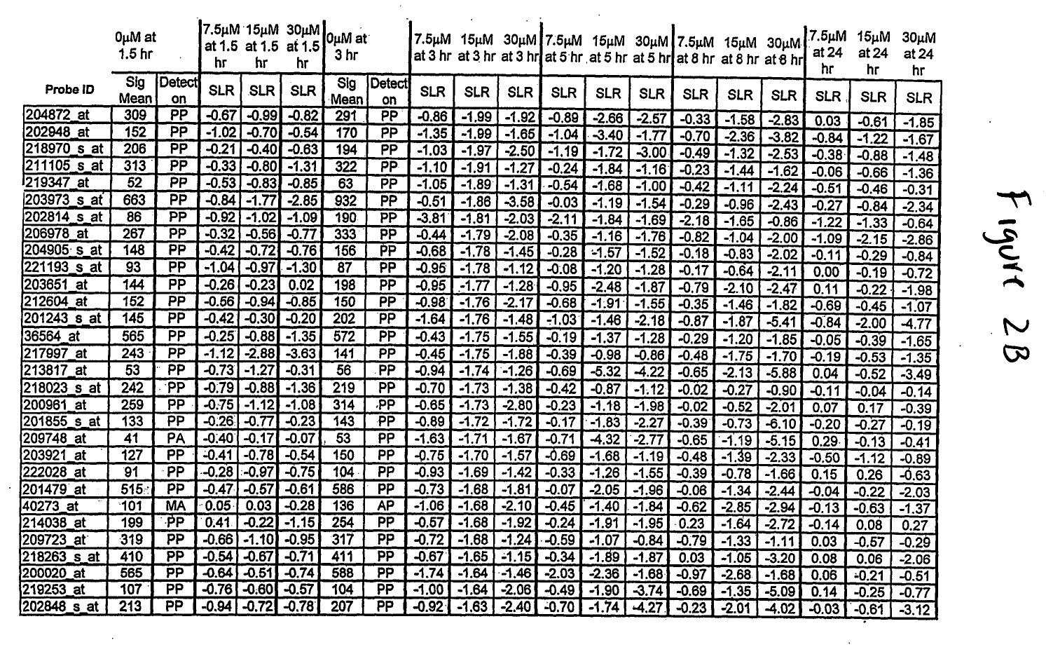

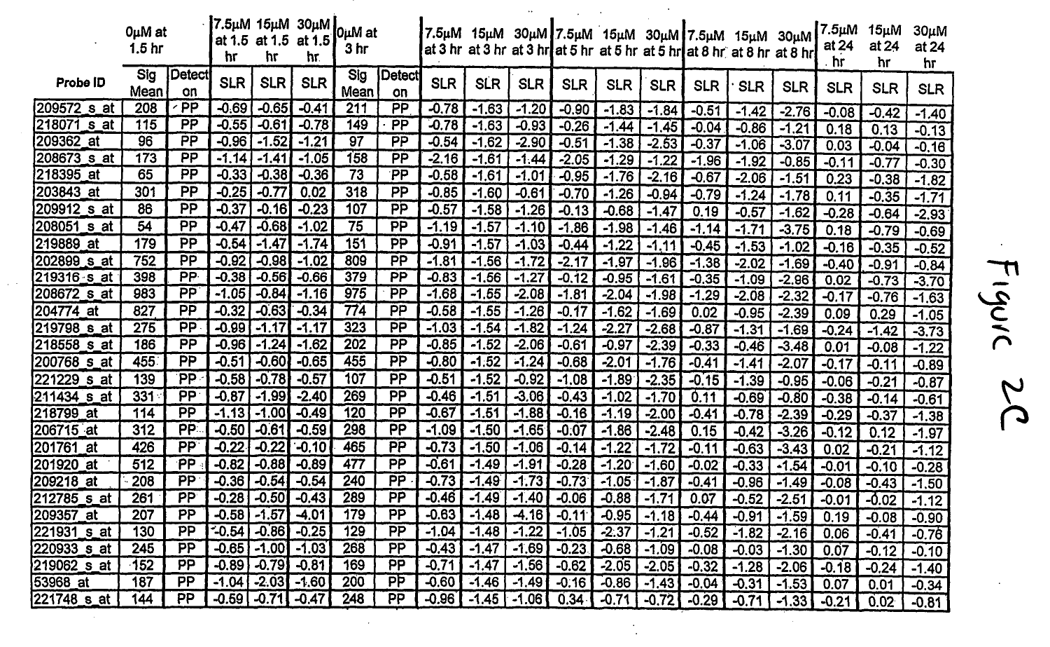

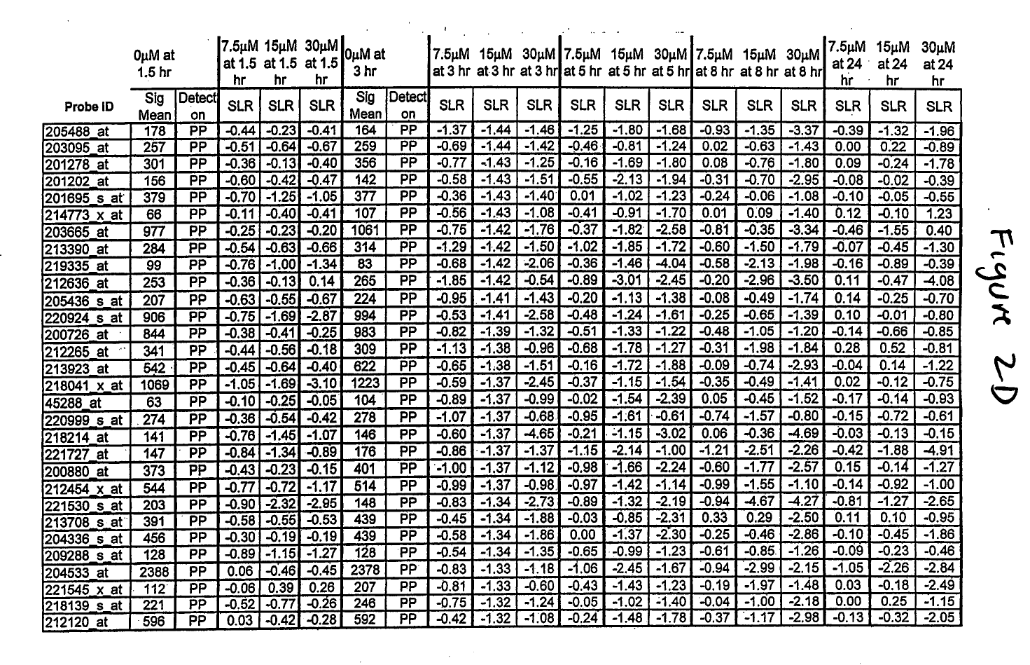

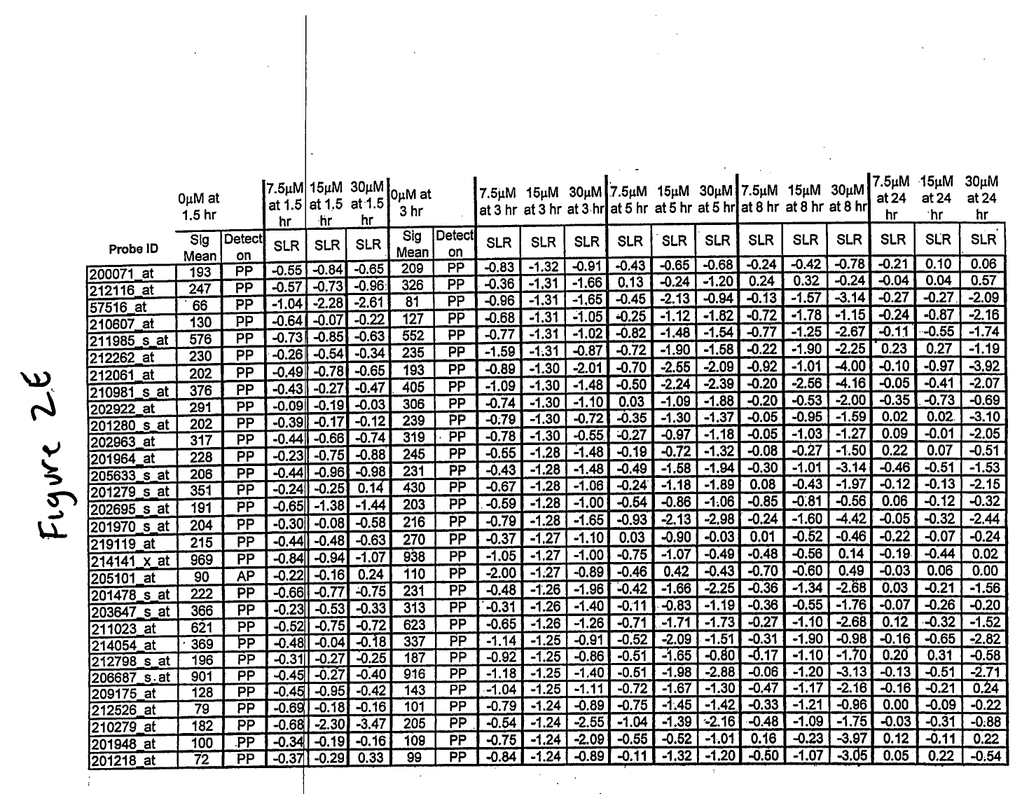

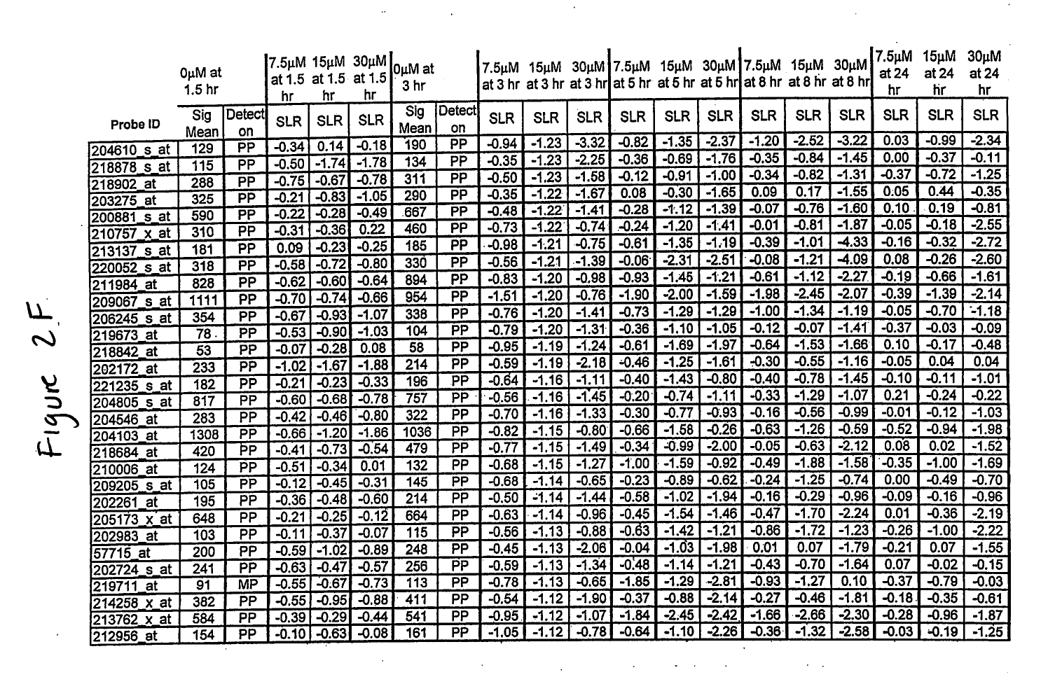

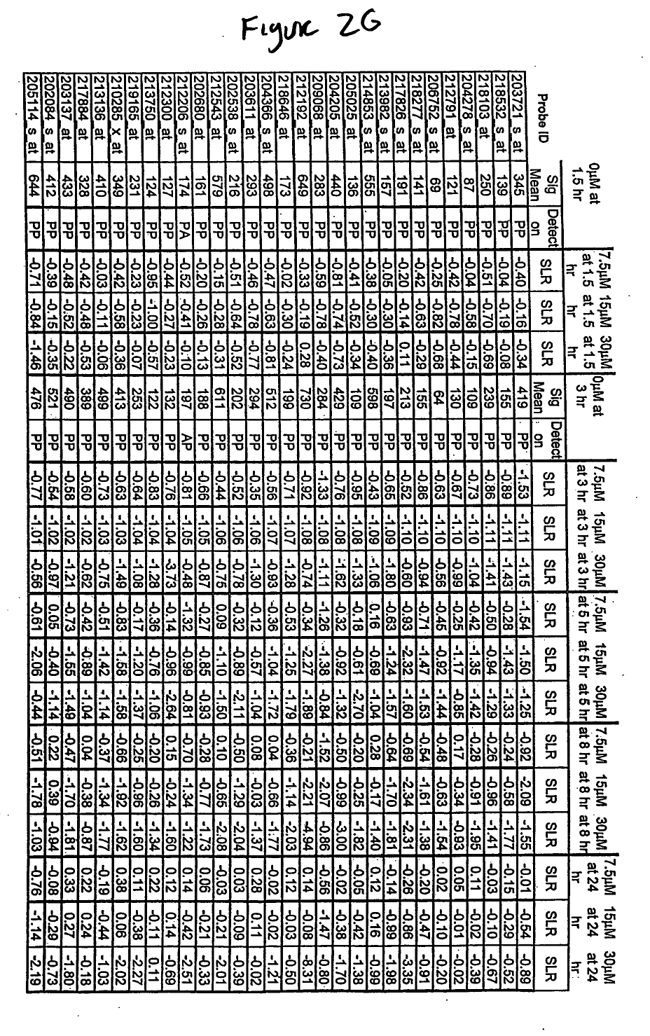

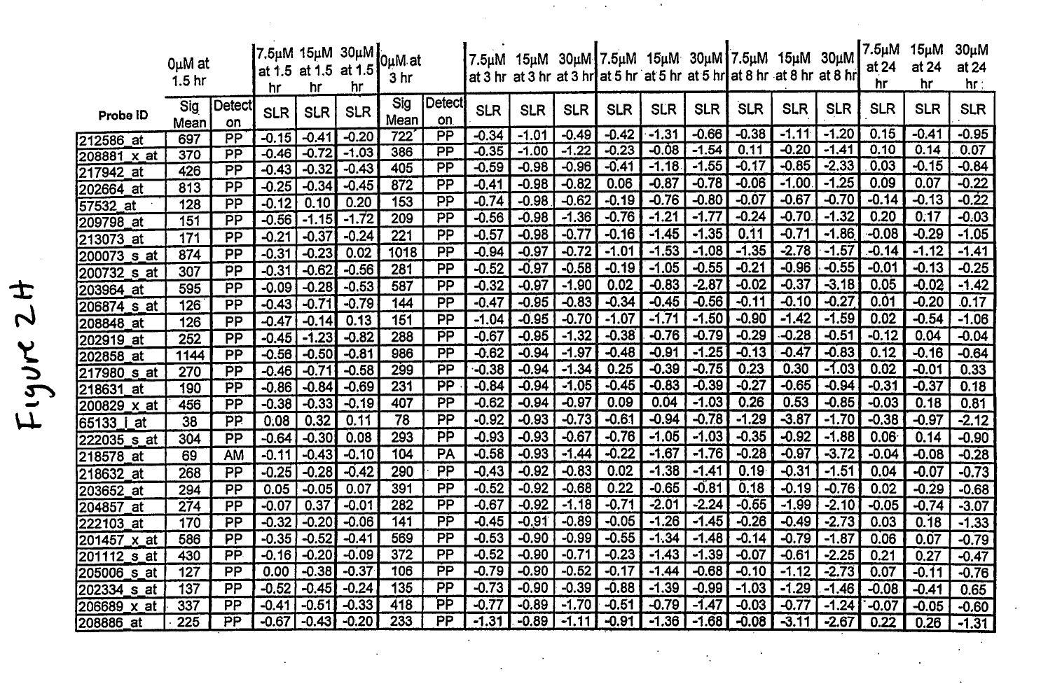

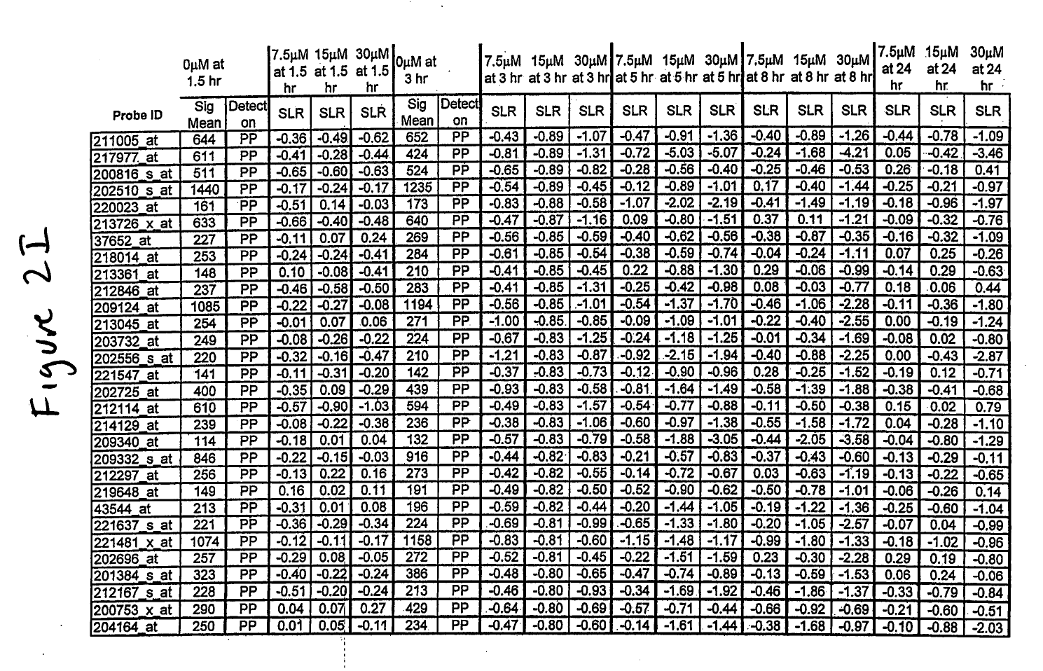

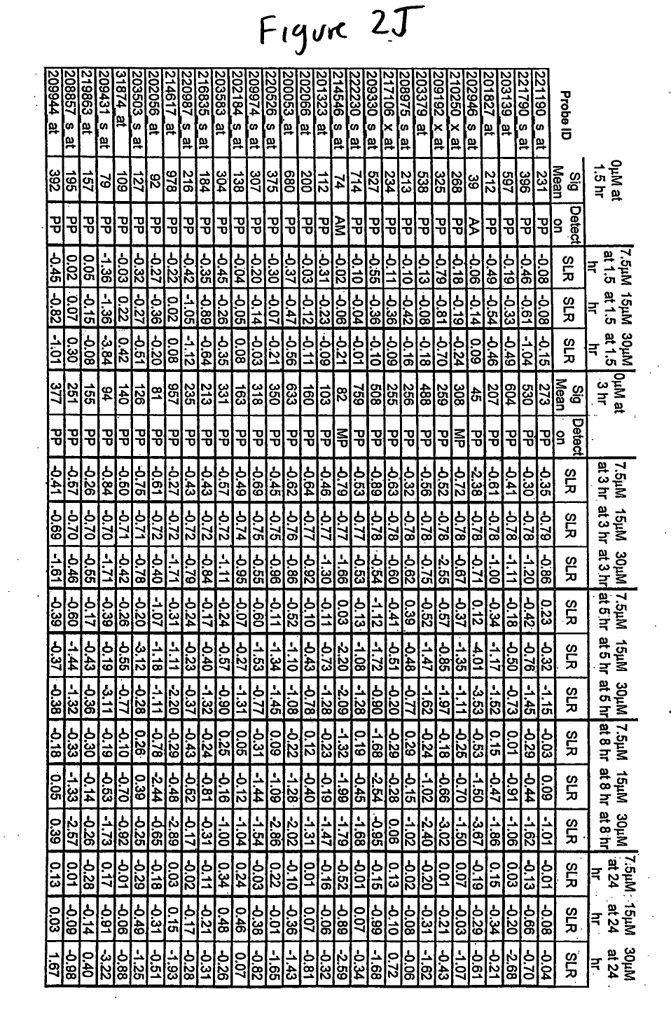

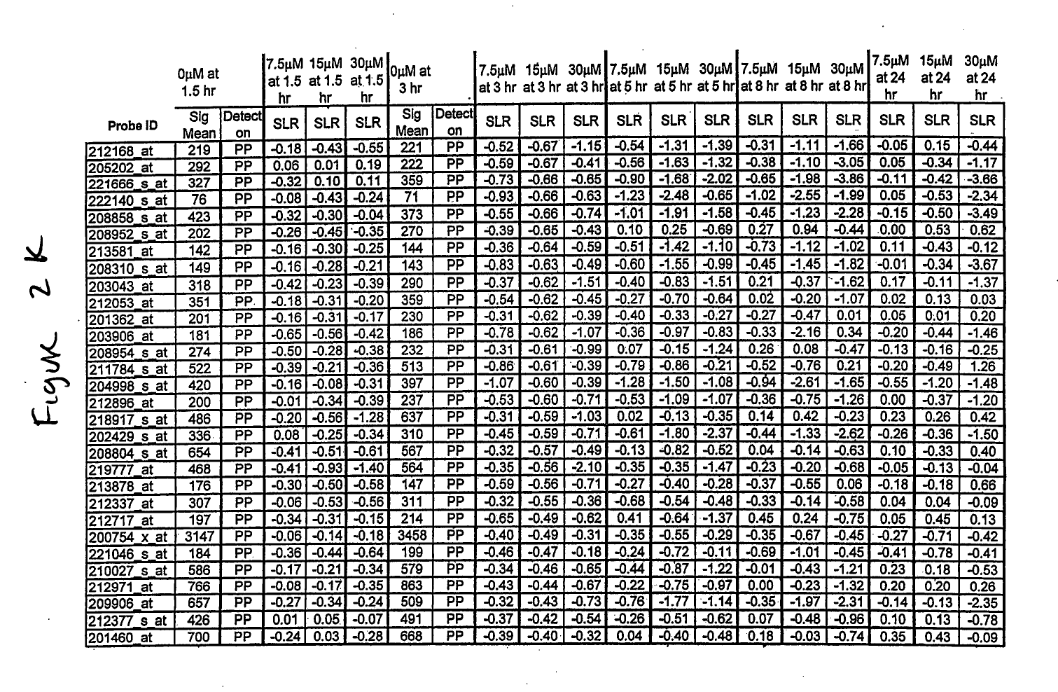

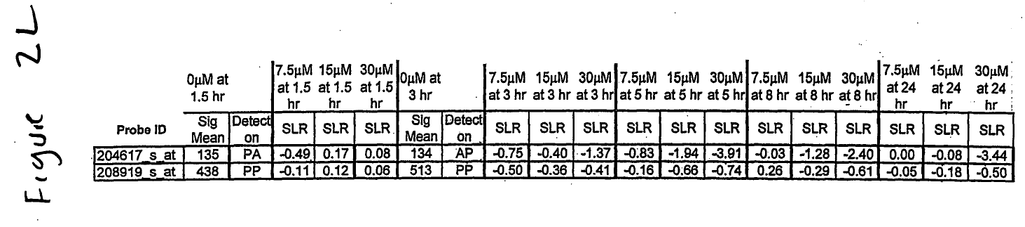

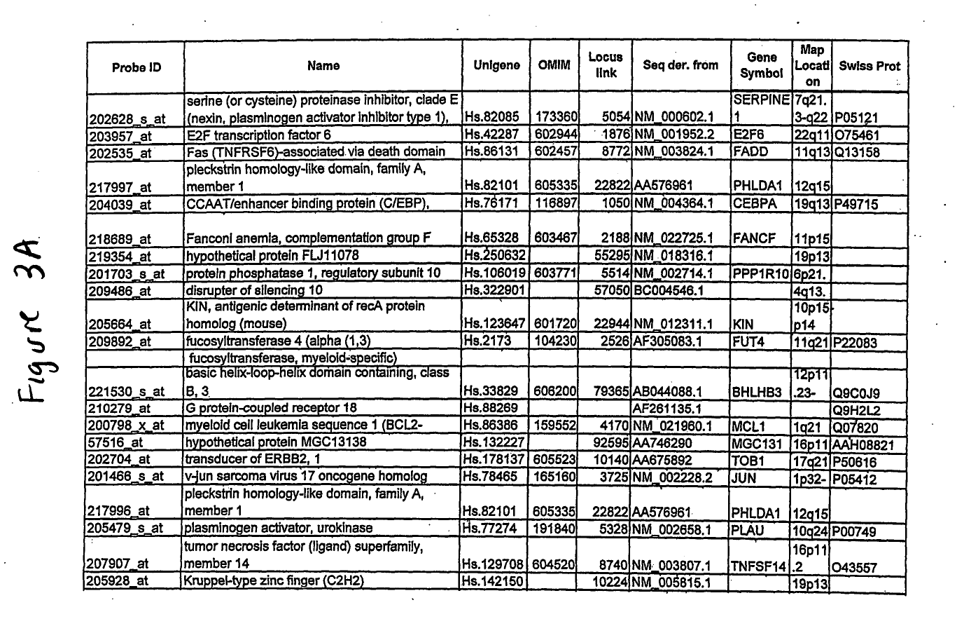

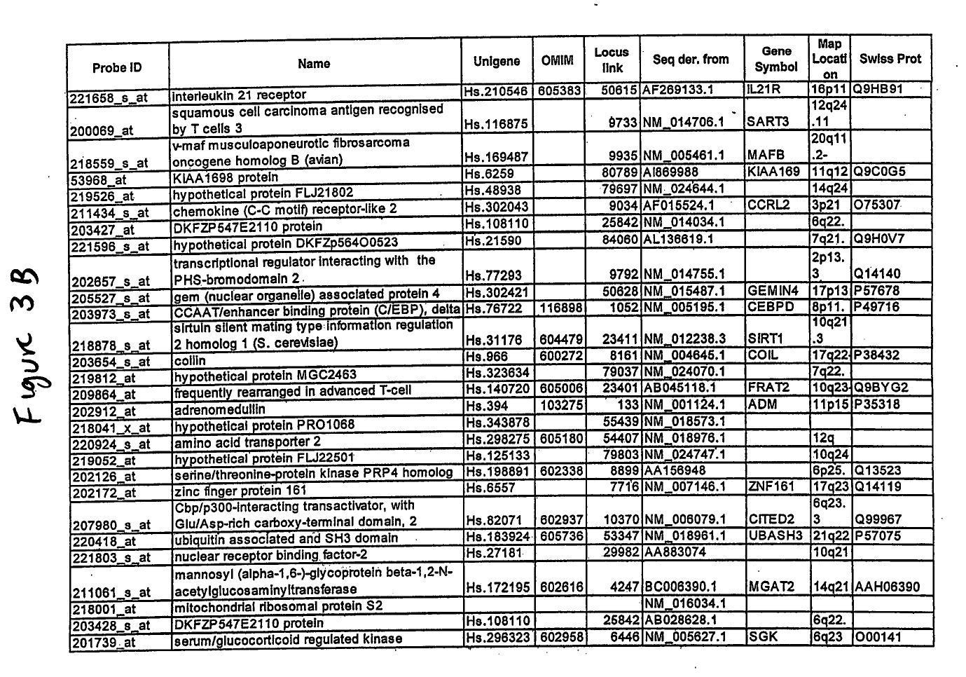

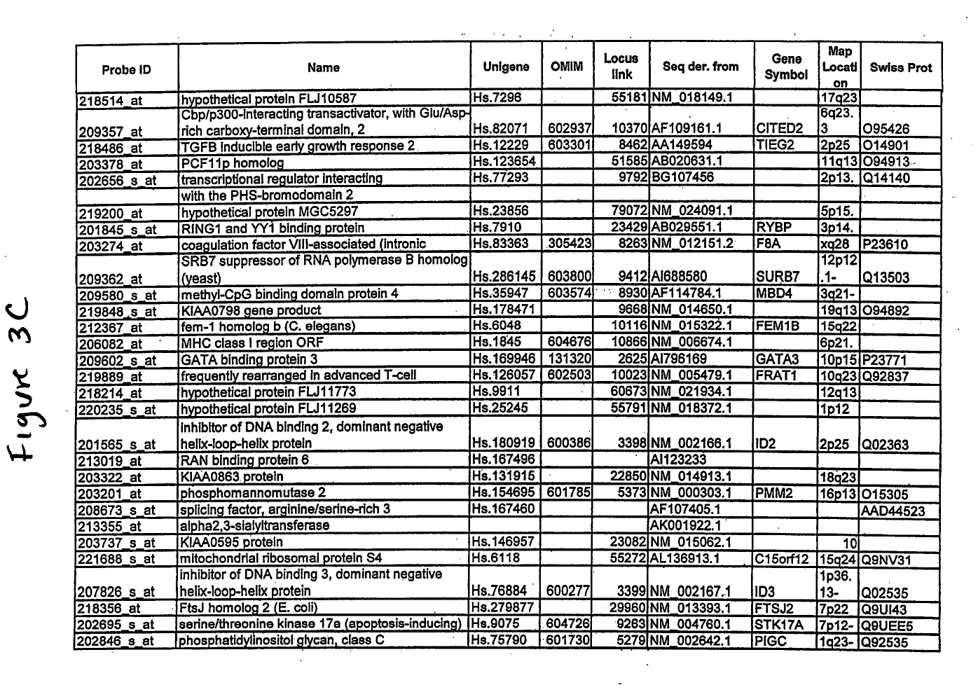

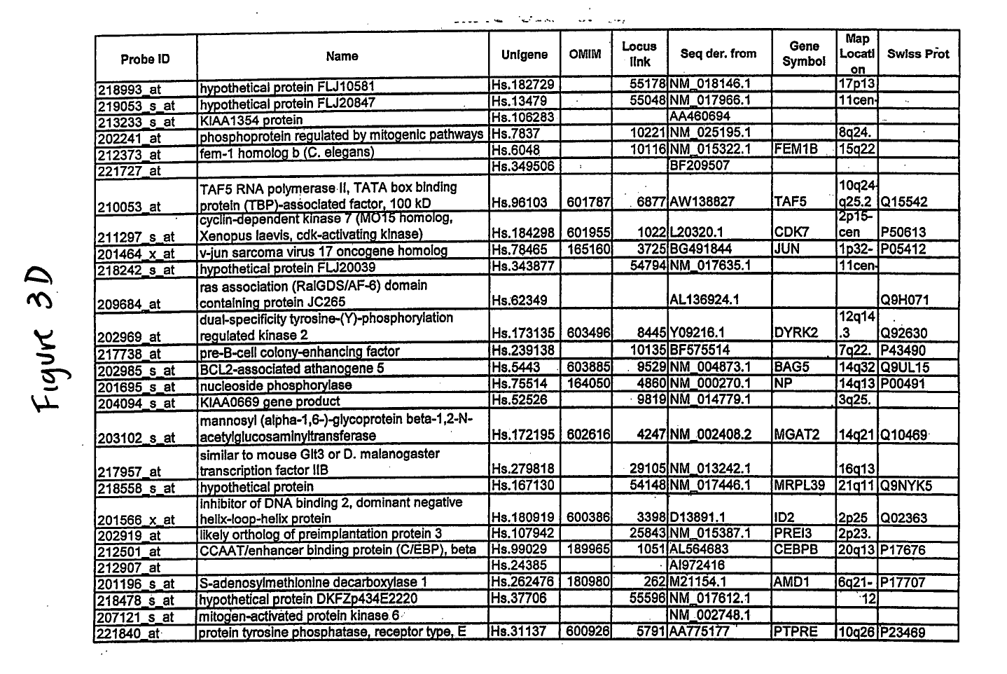

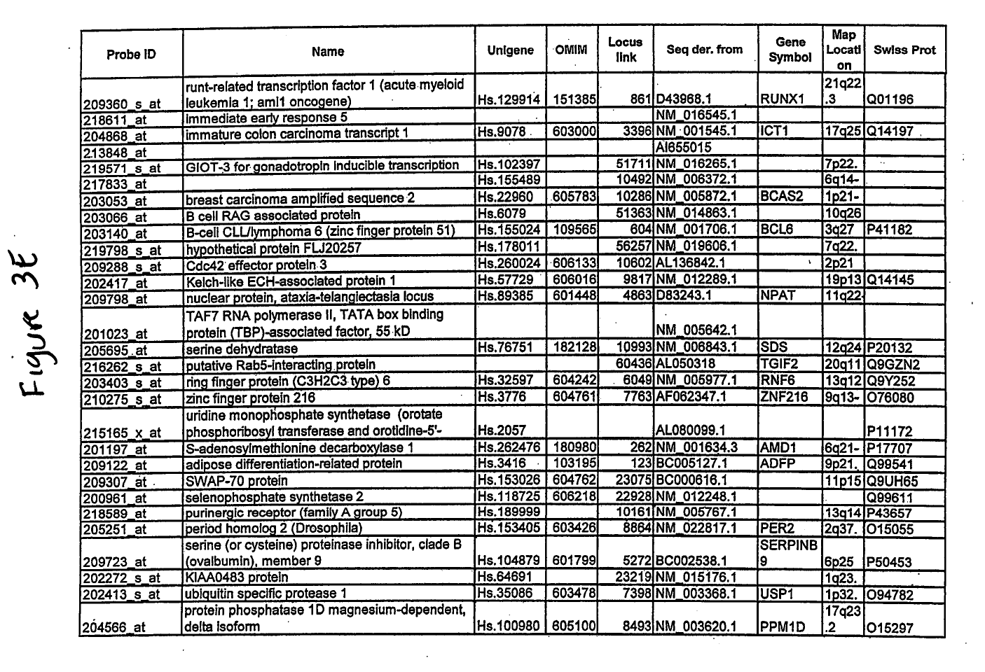

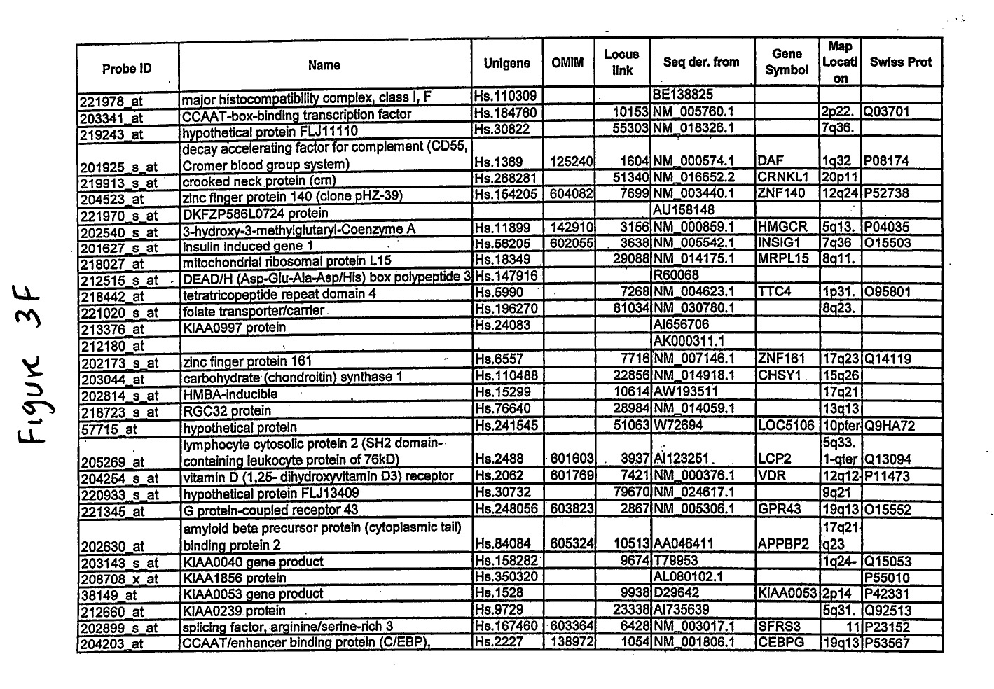

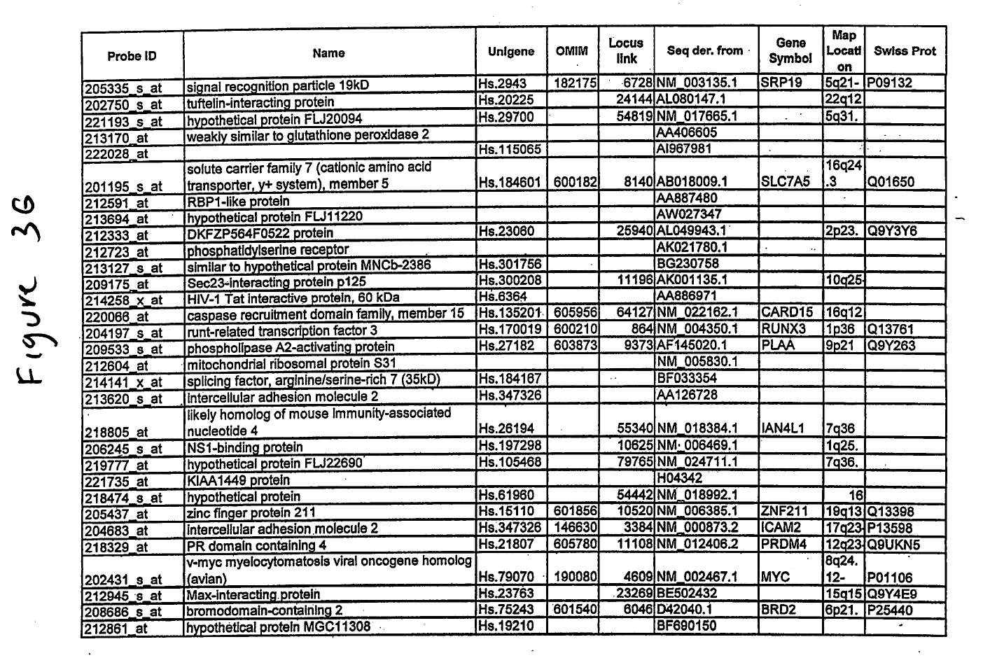

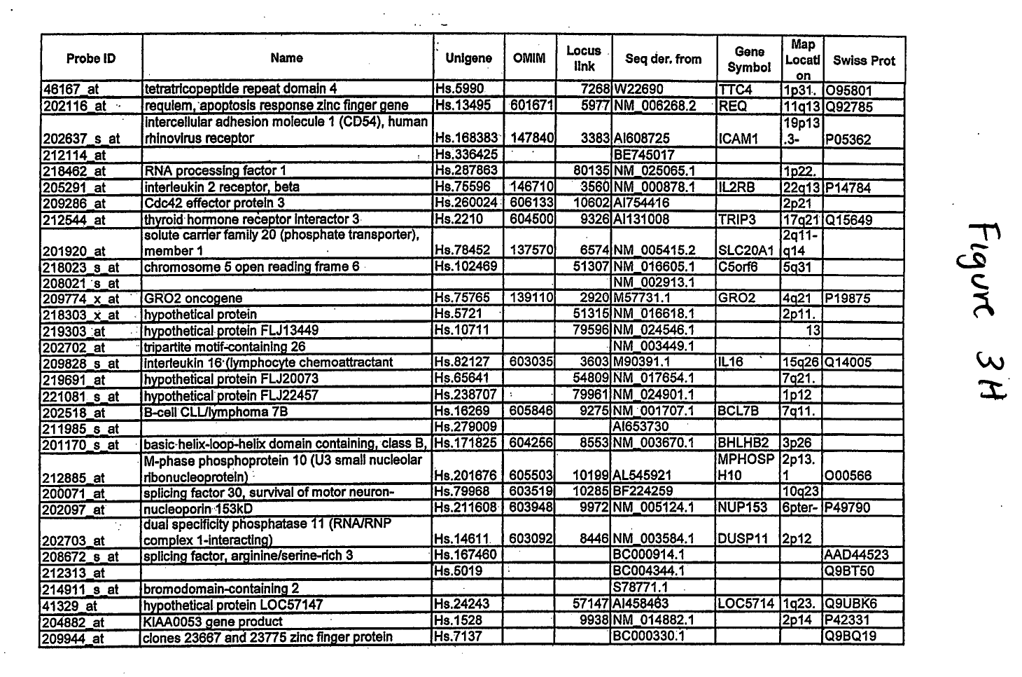

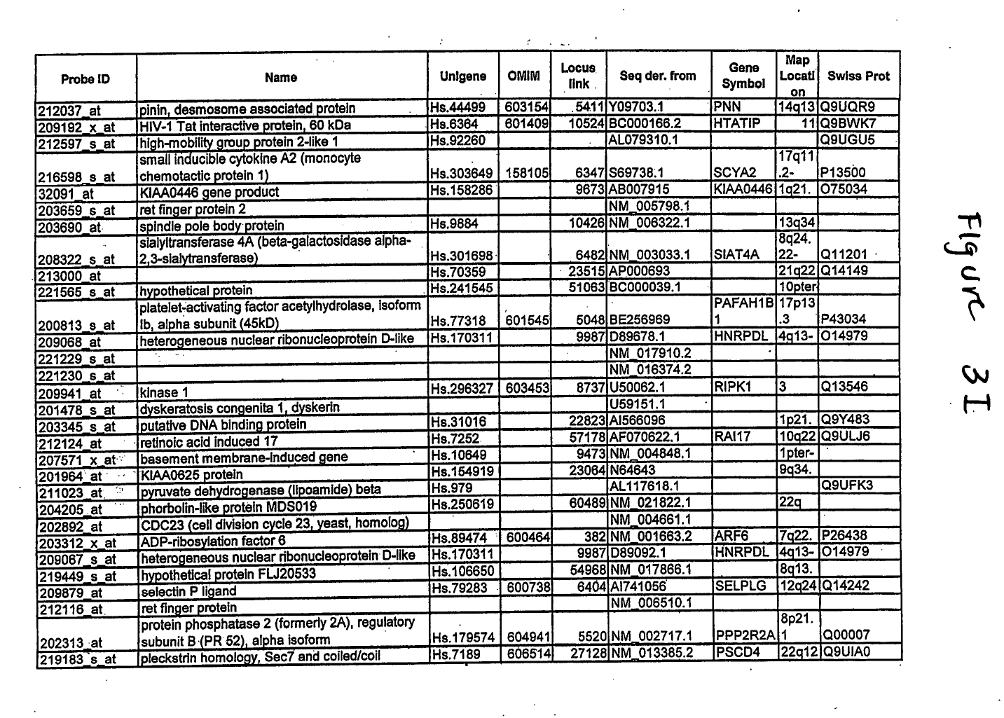

FIG. 3 shows the identity of the genes corresponding to the probes on the Affymetrix Chips whose expression is down regulated at 1.5 hours (i.e. those probes identified in FIG. 1)

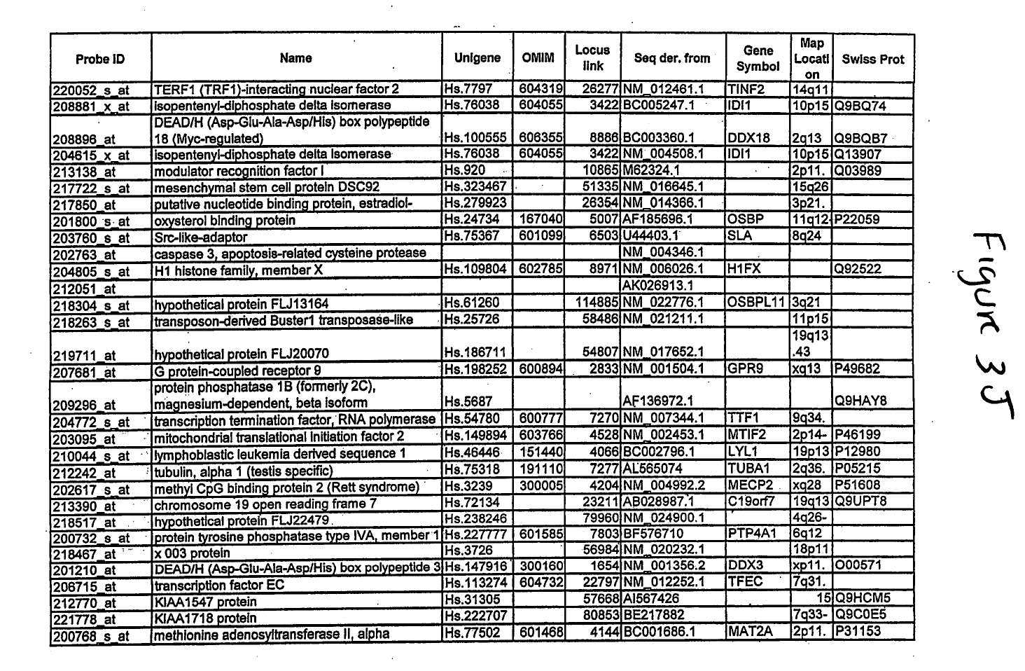

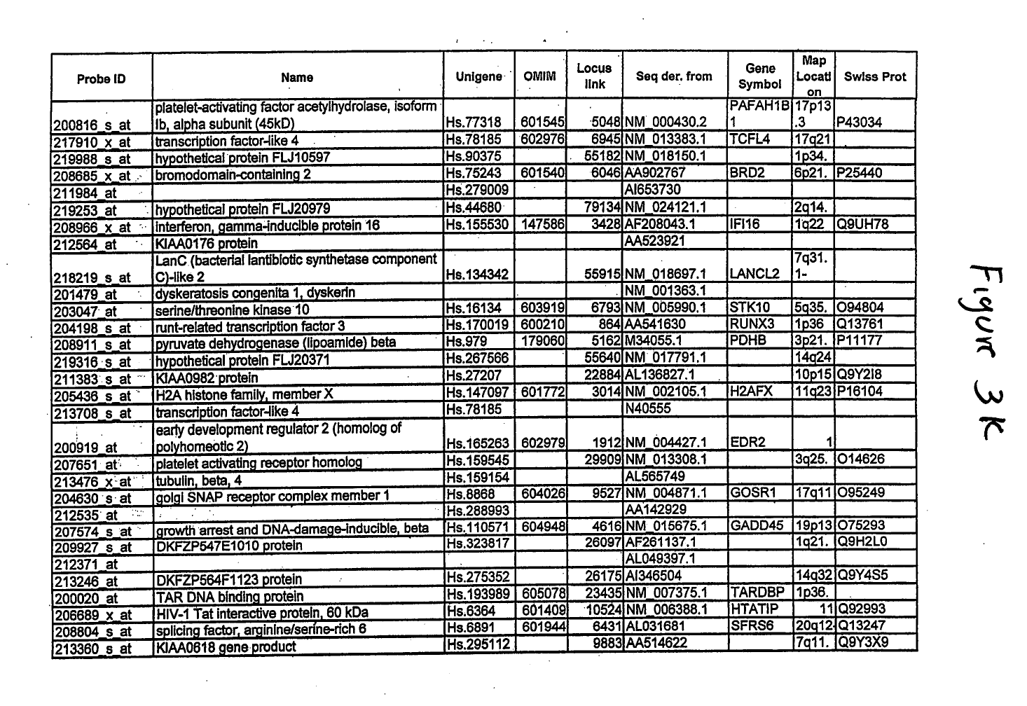

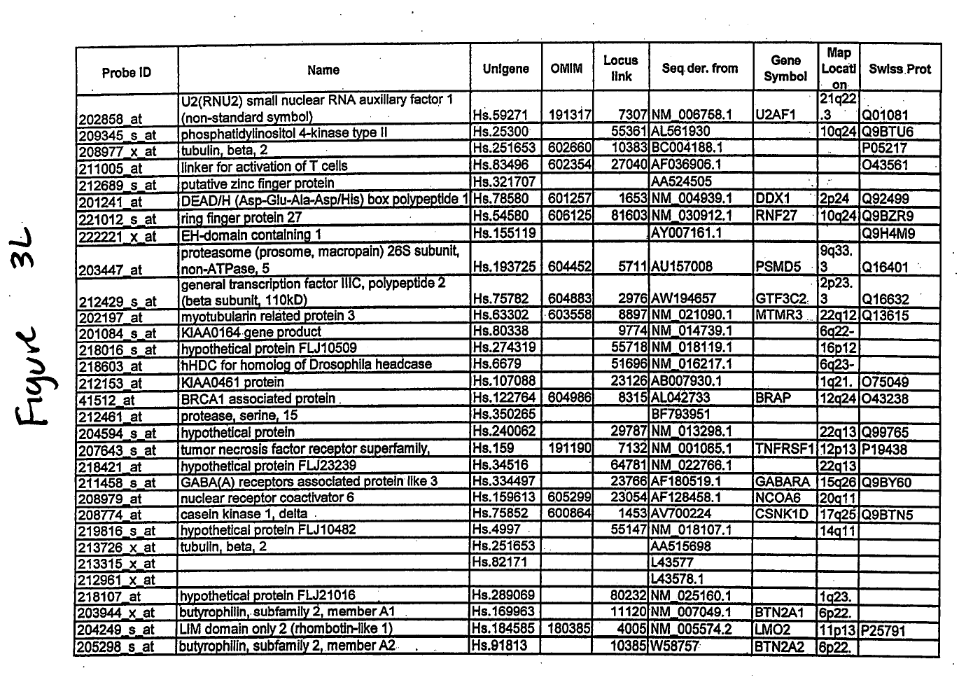

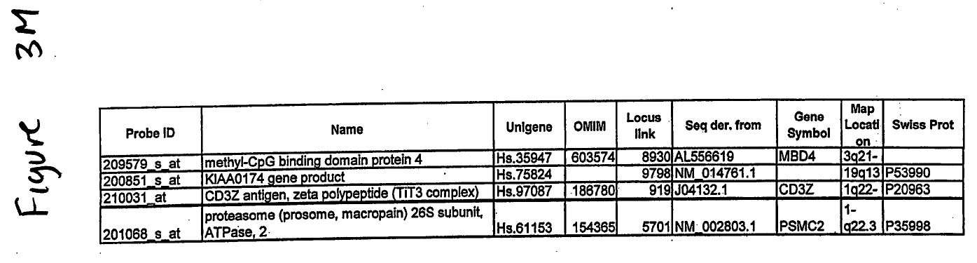

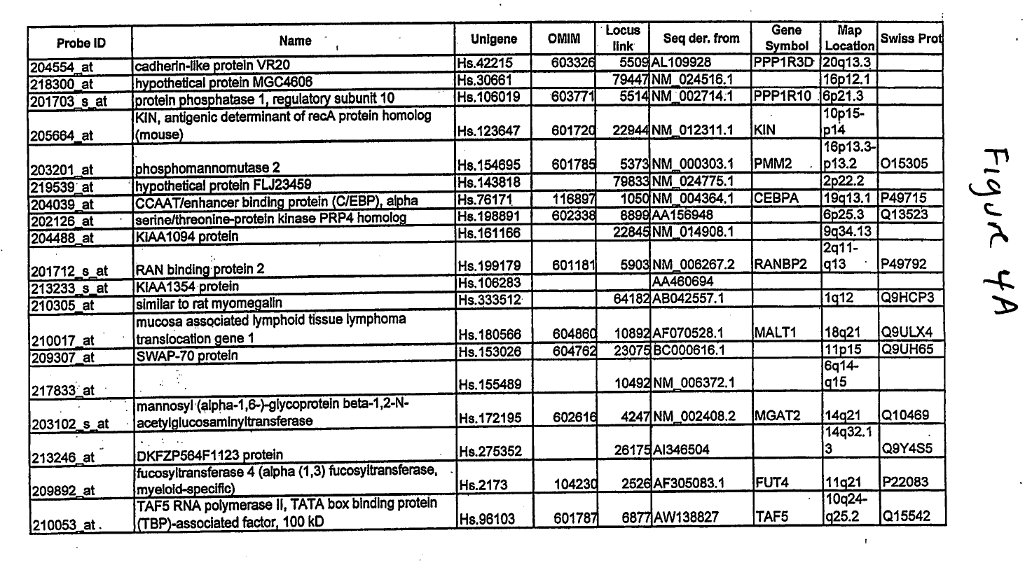

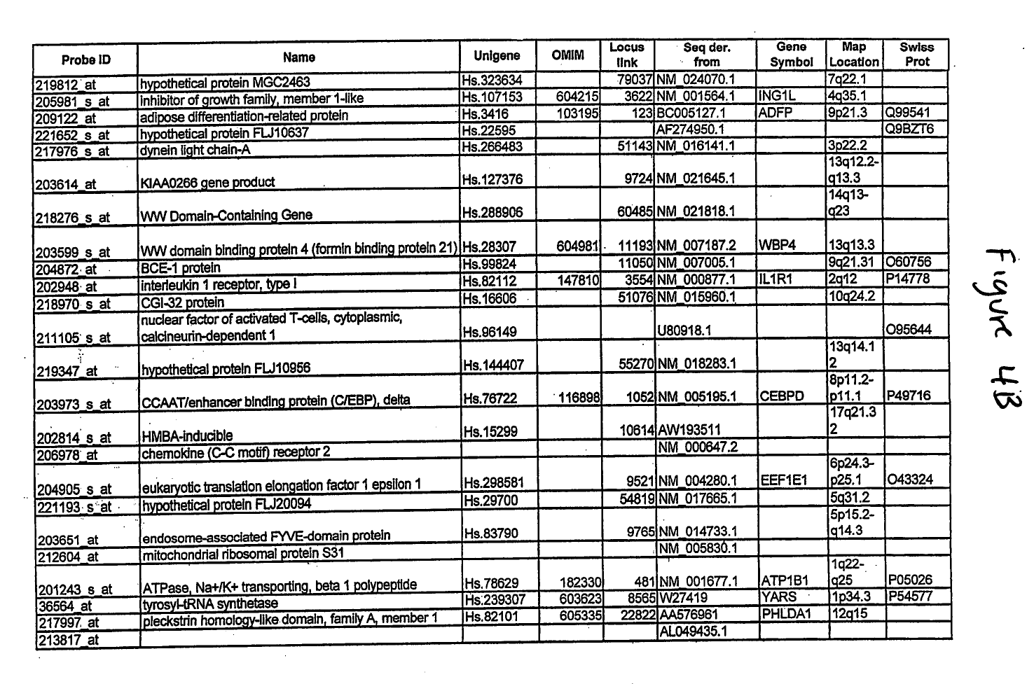

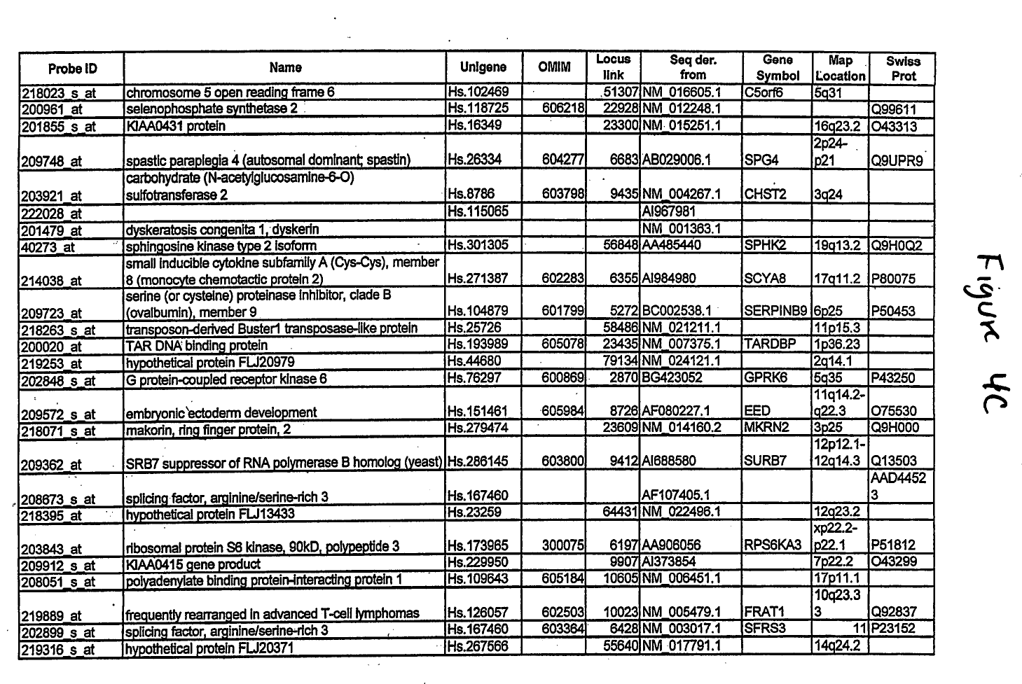

FIG. 4 shows the identity of the genes corresponding to the probes on the Affymetrix Chips whose expression is down regulated at 3 hours (i.e. those probes identified in FIG. 2).

Expression data obtained in microarrays using samples derived from HT29 cells treated with CYC202 at 7.5, 15 and 30 micromolar for 1.5, 3, 5, 8 and 24 hrs is given in the following Figures.

FIG. 5 shows the results of CYC202 treatment of HT29 cells, identifying those genes whose expression is significantly down regulated at 1.5 hr along with the corresponding data for expression of those genes at later time points.

FIG. 6 shows the results of CYC202 treatment of HT29 cells, identifying those genes whose expression is significantly down regulated at 3 hr along with the corresponding data for expression of those genes at later time points.

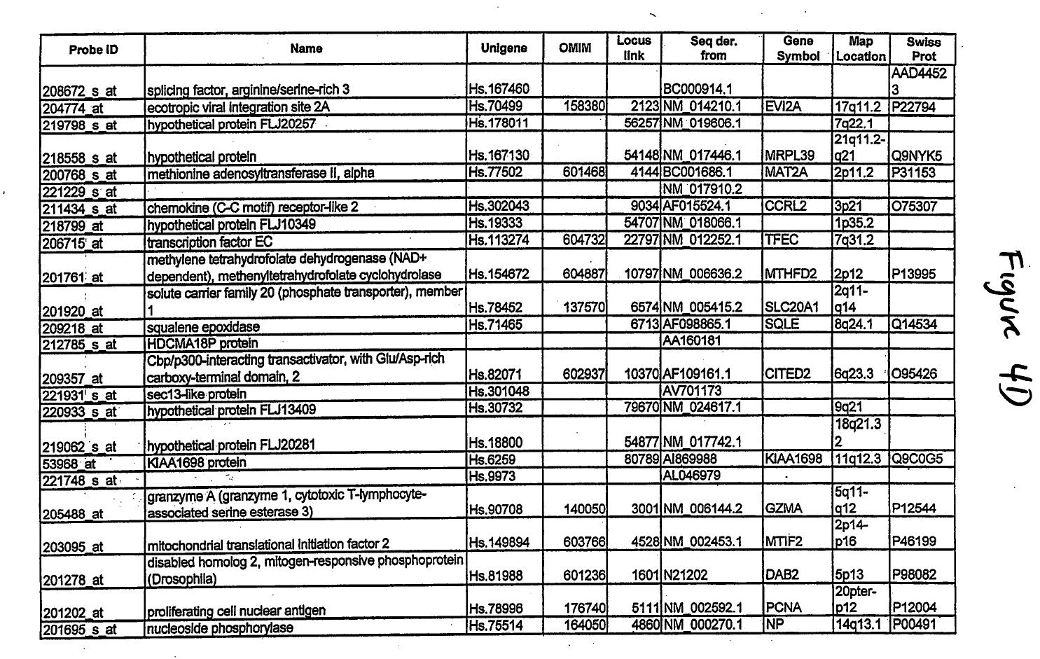

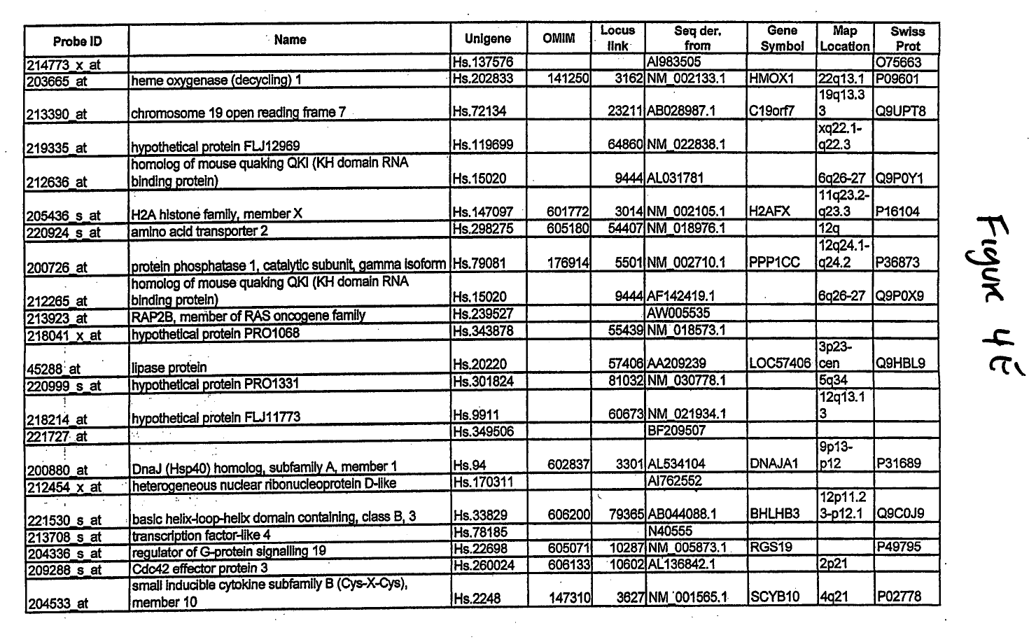

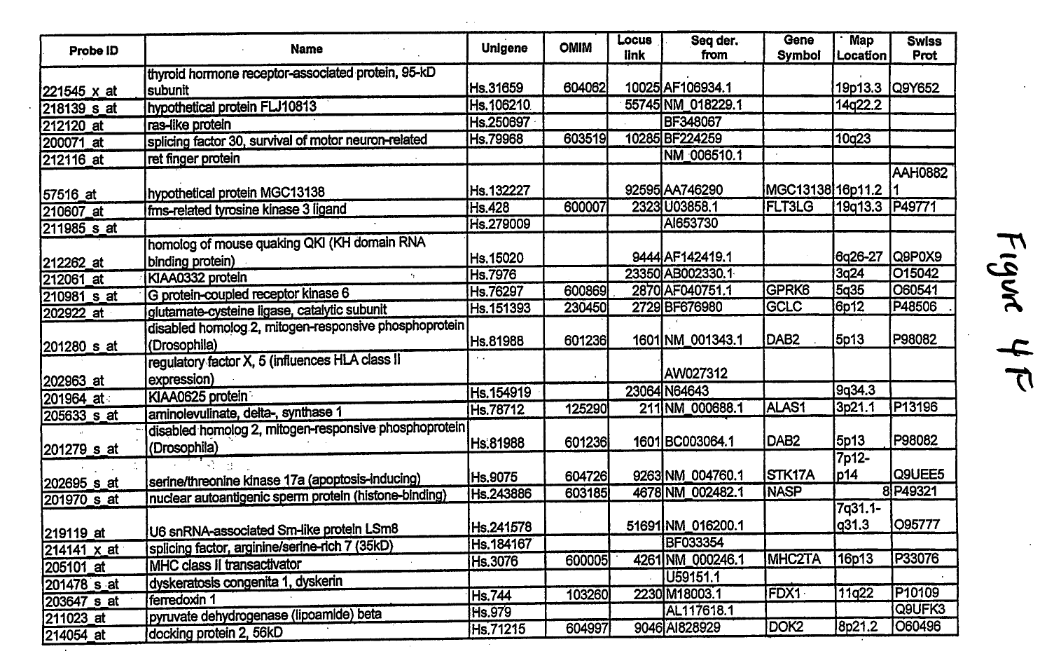

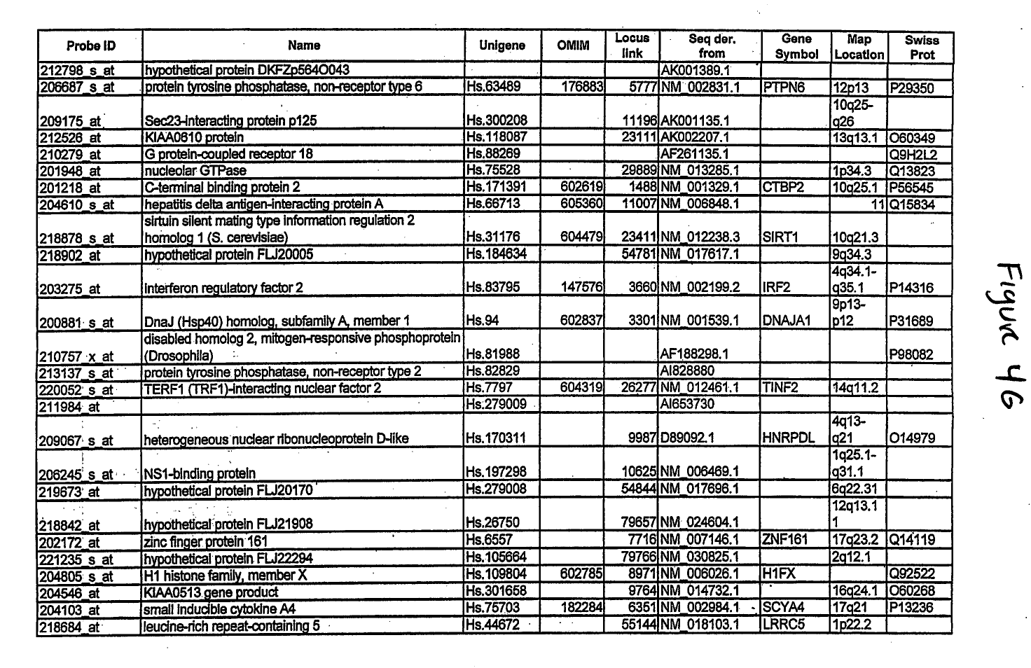

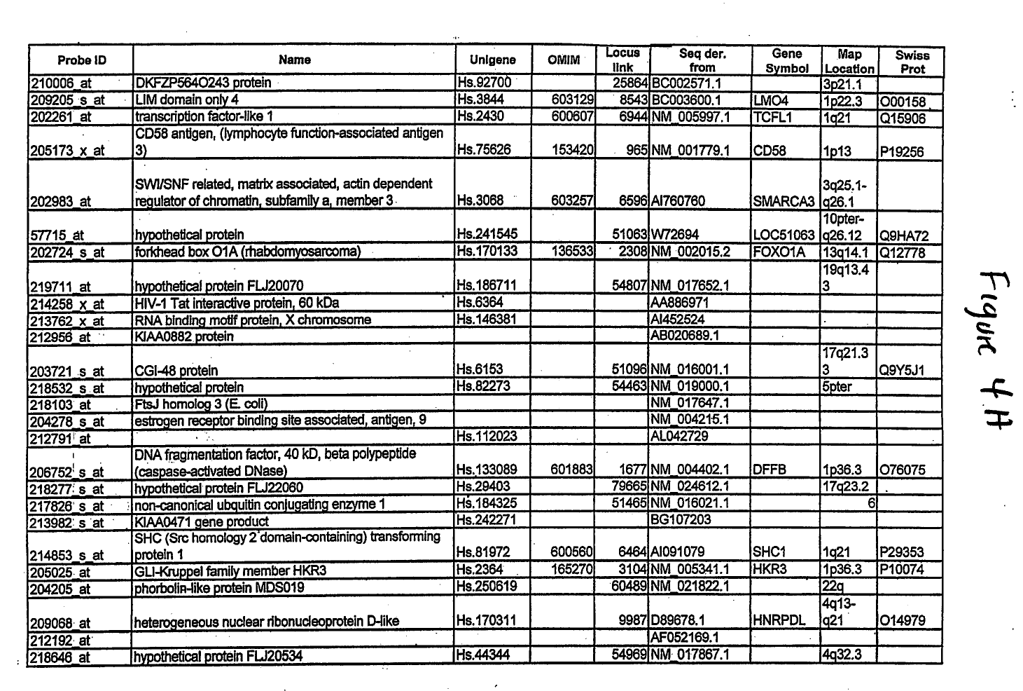

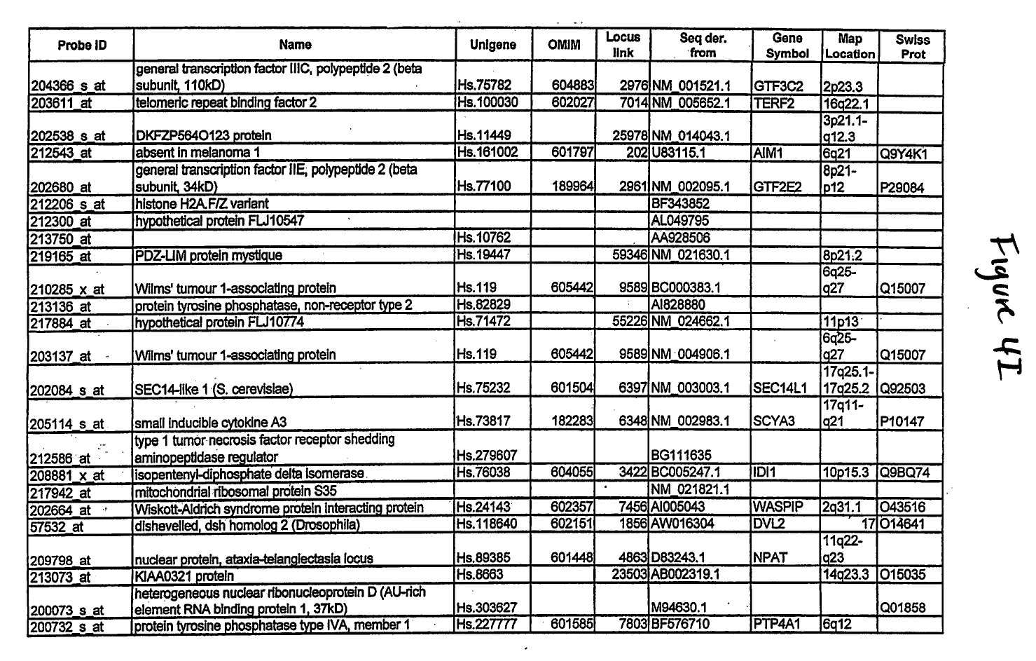

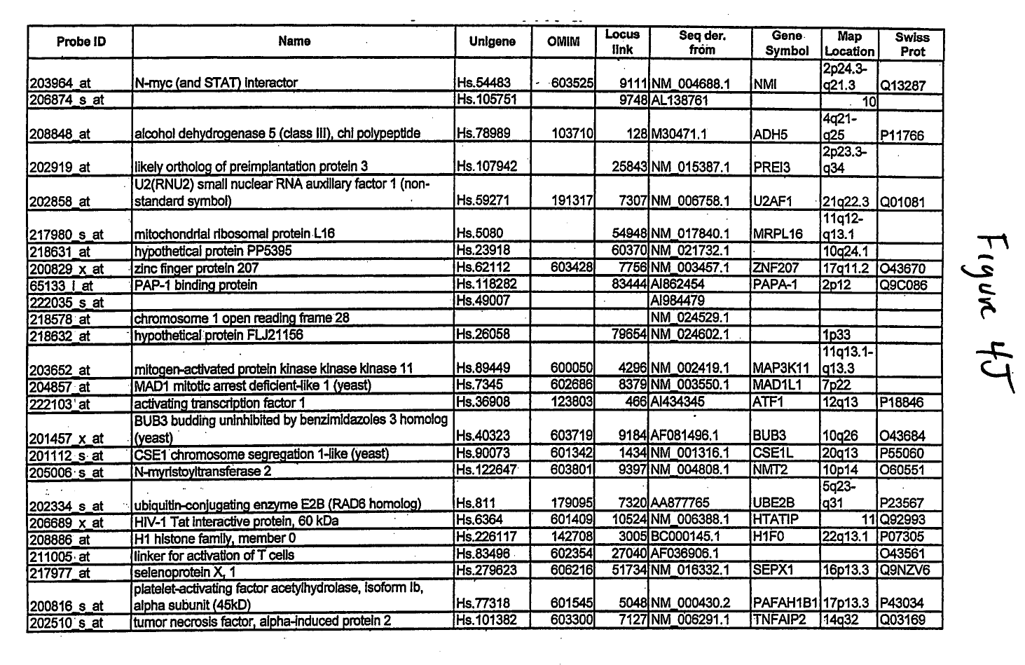

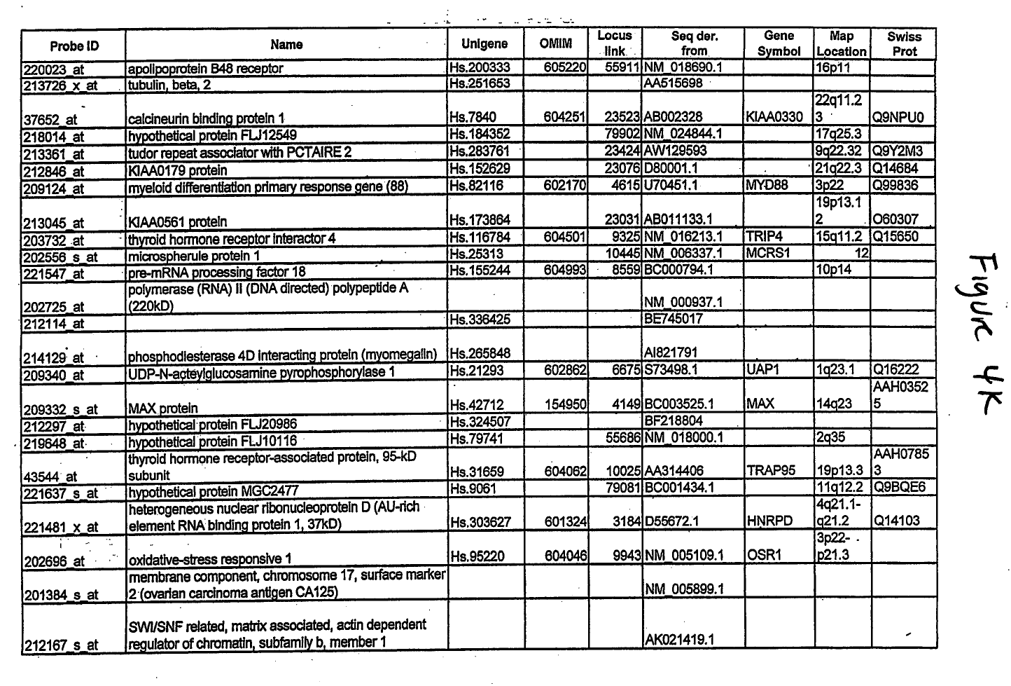

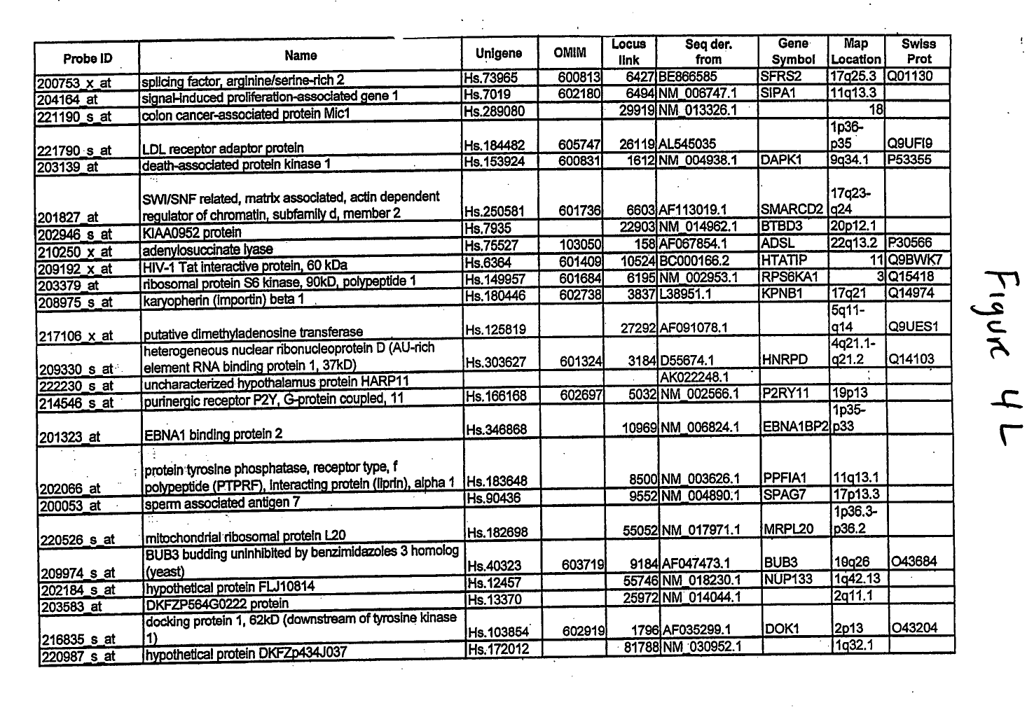

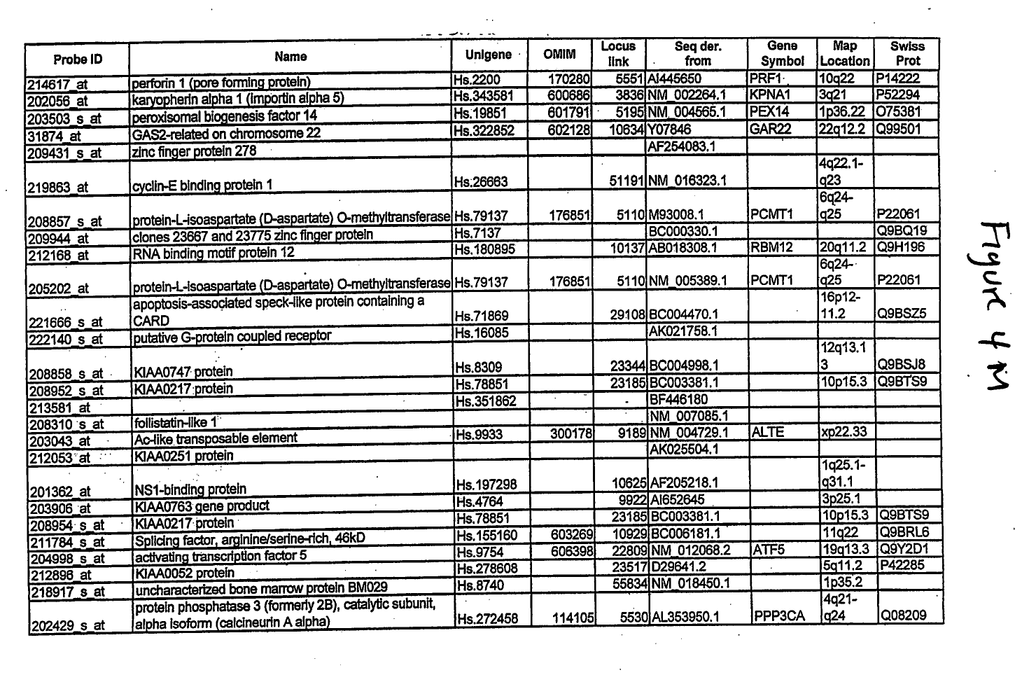

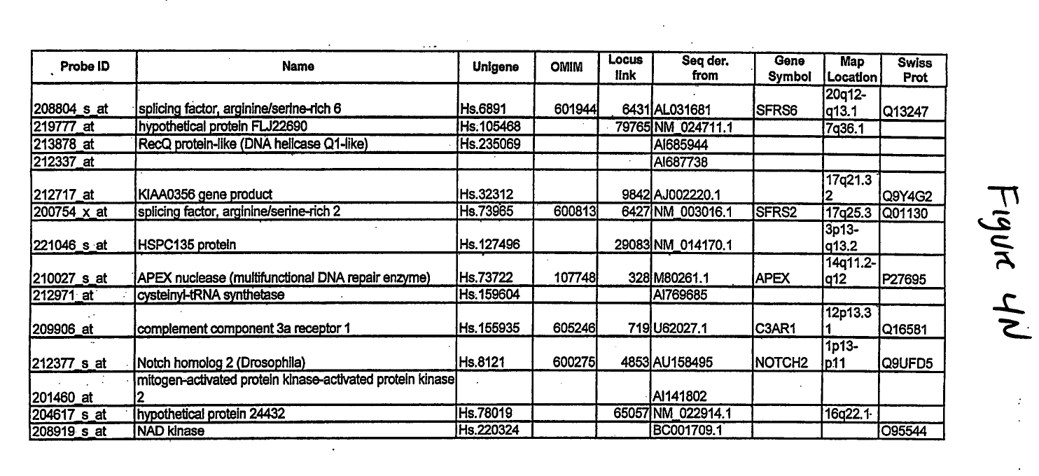

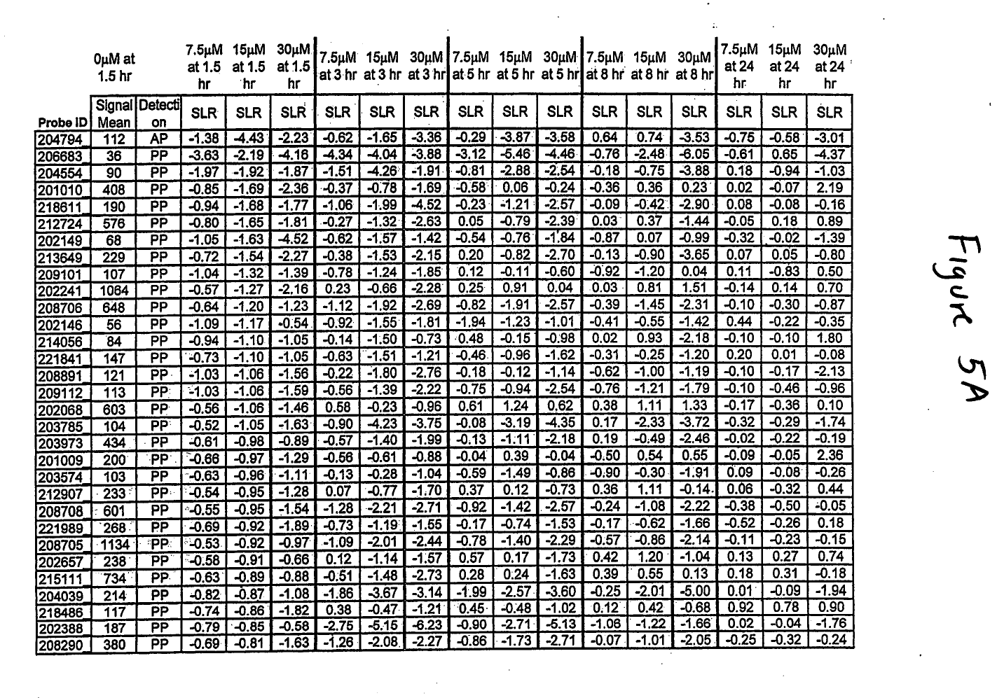

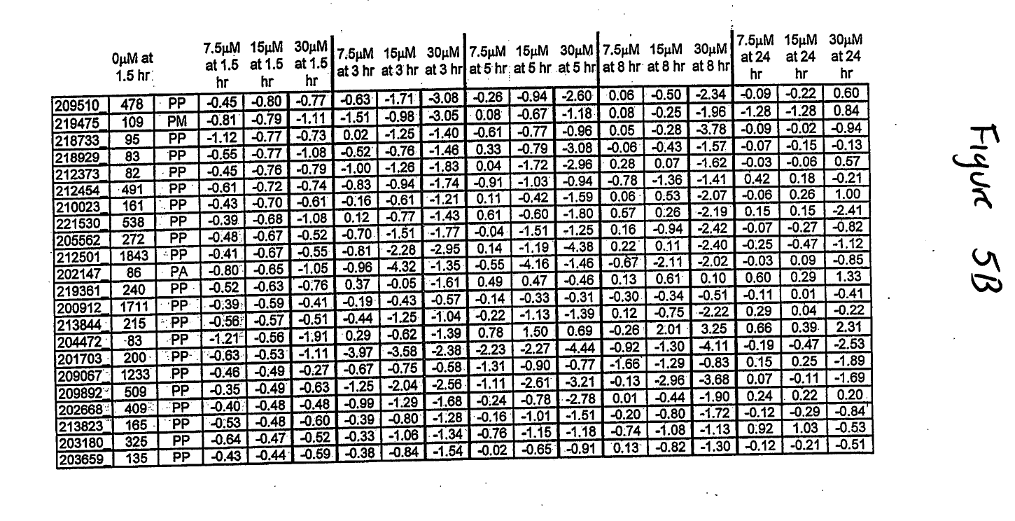

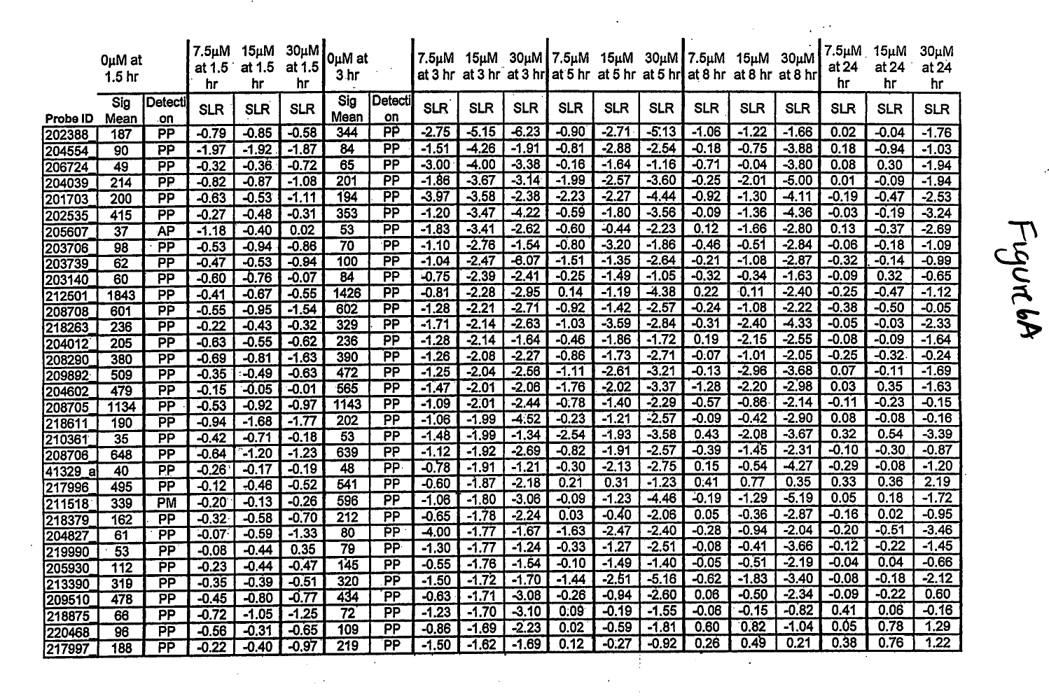

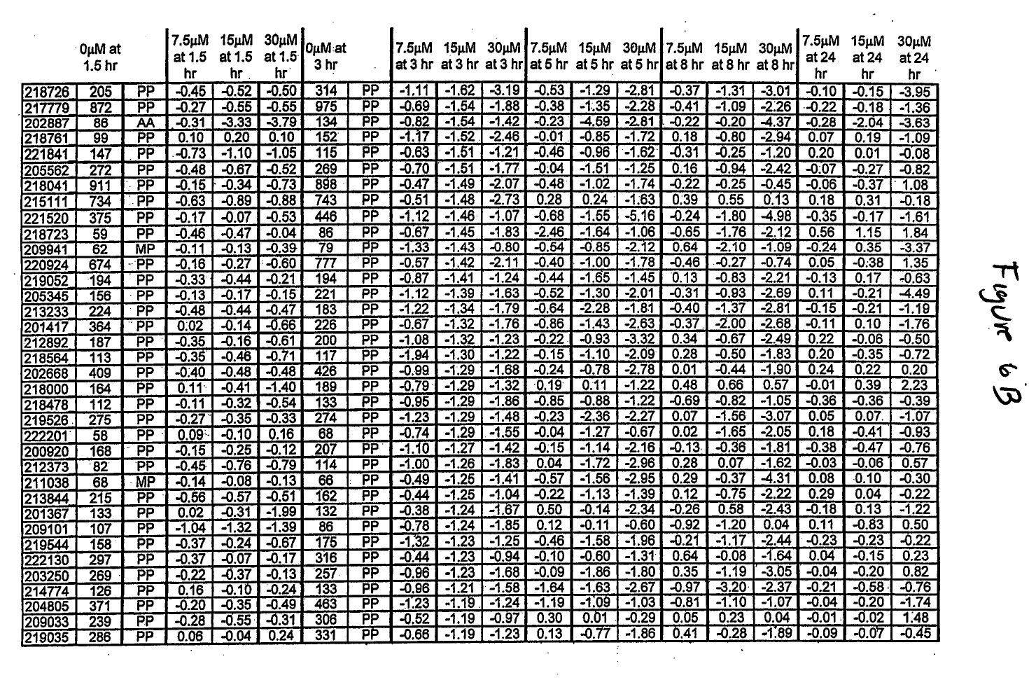

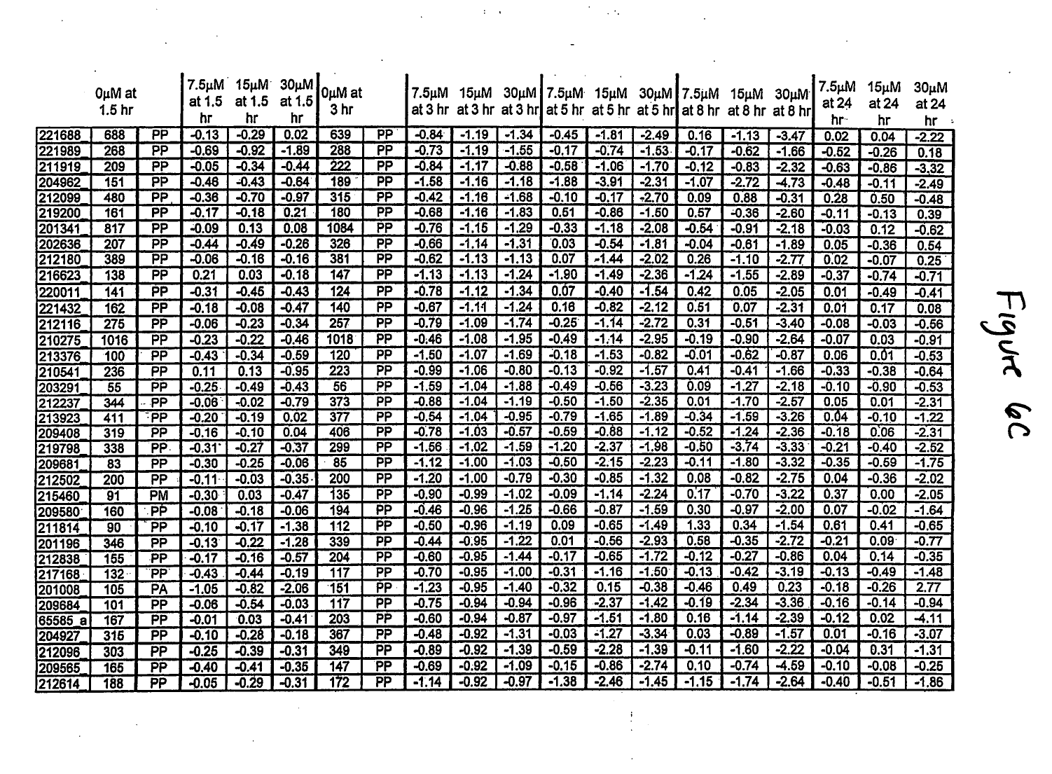

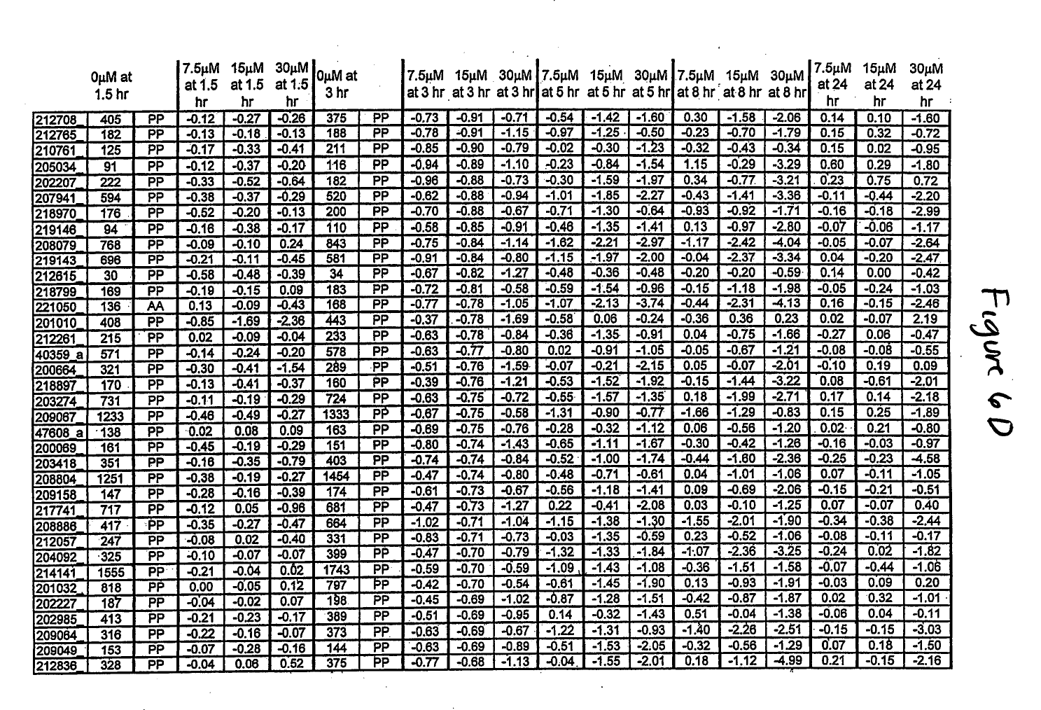

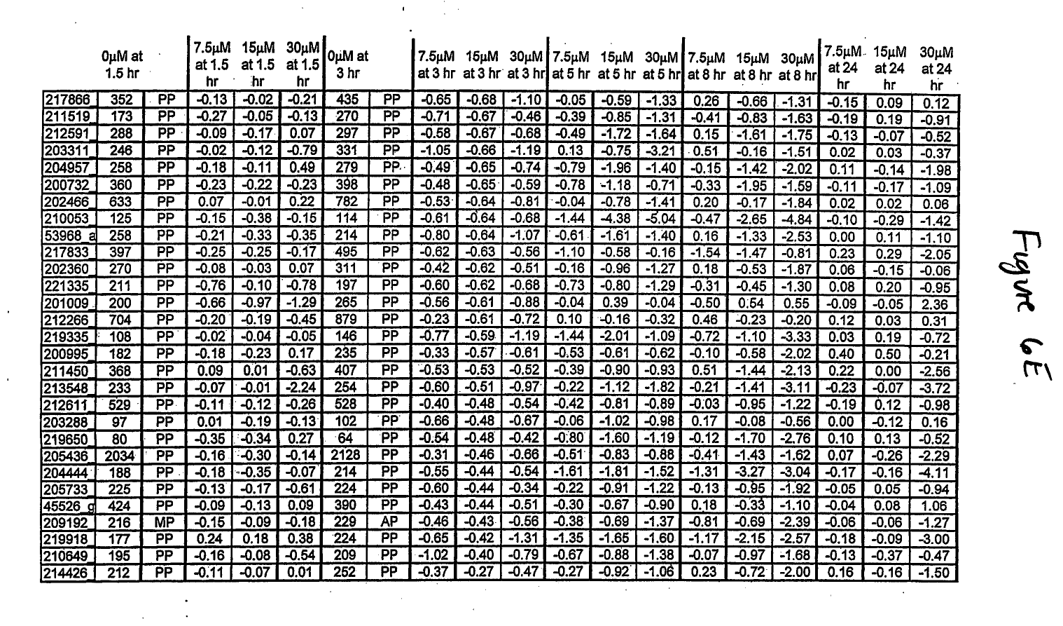

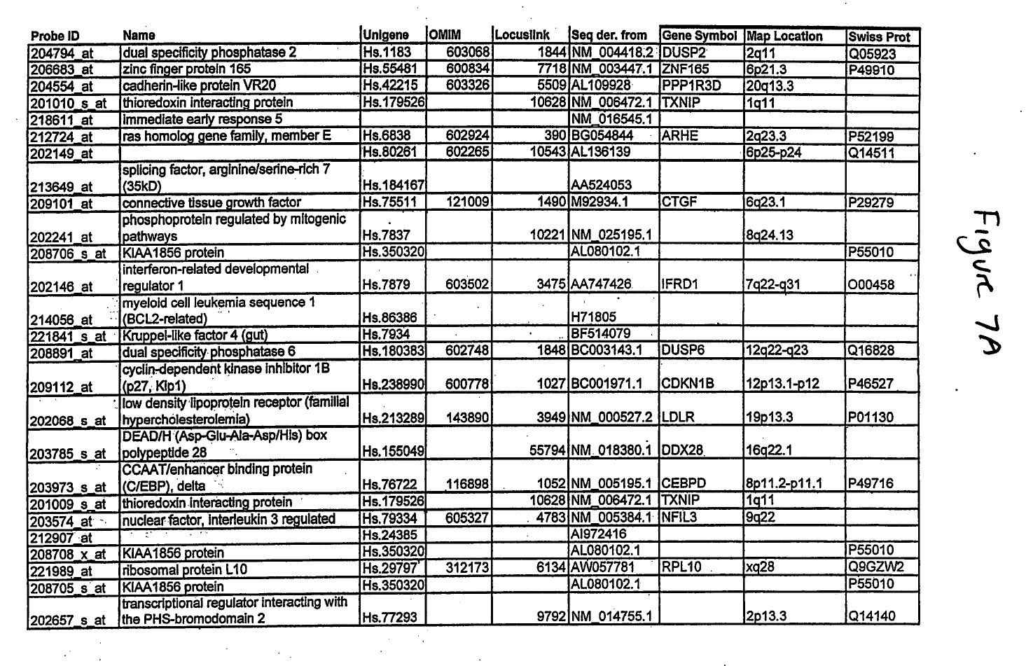

FIG. 7 shows the identity of the genes corresponding to the probes on the Affymetrix Chips whose expression is down regulated at 1.5 hours (i.e. those probes identified in FIG. 5).

FIG. 8 shows the identity of the genes corresponding to the probes on the Affymetrix Chips whose expression is down regulated at 3 hours (i.e. those probes identified in FIG. 6).

Expression data obtained in microarrays using samples derived from A549 cells treated with CYC202 at 15, 45 and 75 micromolar for 2, 4, 8 and 24 hrs is given in the following Figures.

FIG. 9 shows the results of CYC202 treatment of A549 cells, identifying those genes whose expression is significantly down regulated at 2 hr along with the corresponding data for expression of those genes at later time points.

FIG. 10 shows the results of CYC202 treatment of A549 cells, identifying those genes whose expression is significantly down regulated at 4 hr along with the corresponding data for expression of those genes at later time points.

FIG. 11 shows the identity of the genes corresponding to the probes on the Affymetrix Chips whose expression is down regulated at 2 hours (i.e. those probes identified in FIG. 9).

FIG. 12 shows the identity of the genes corresponding to the probes on the Affymetrix Chips whose expression is down regulated at 4 hours (i.e. those probes identified in FIG. 10).

### Example 2

### Confirming Microarray Data Using Real-Time Quantitative PCR

Real-Time Quantitative PCR (QPCR)

Total RNA samples obtained from the same three cell lines were used for verification of gene expression levels observed on the microarrays. Quantitative RT-PCR was performed on a Roche LightCycler machine (Roche, UK). Primers were chosen to exclude the possibility of obtaining products arising from trace contamination of the RNA samples with genomic DNA.

In addition, RNA from whole blood samples was analysed by QPCR. Blood from volunteers was collected into Vacutainer Heparin CPT tubes. The blood was then treated with various concentrations of CYC202 or DMSO, and incubated at 37° C. in an incubator in the presence of 5% CO₂ for 1.5 hours, with inverting of the tubes every 30 minutes. The blood was then transferred into PAXgene tubes (PreAnalytiX), inverted several times, and placed at −20° C. after an initial incubation of 2 hours at room temperature to allow lysis of the cells. When ready for analysis, blood was left to thaw at room temperature for approximately 2 hours, and then processed according to the manufacturer's instructions. The optional DNase digestion step was included. RNA samples were quantified and then used directly in QPCR assays.

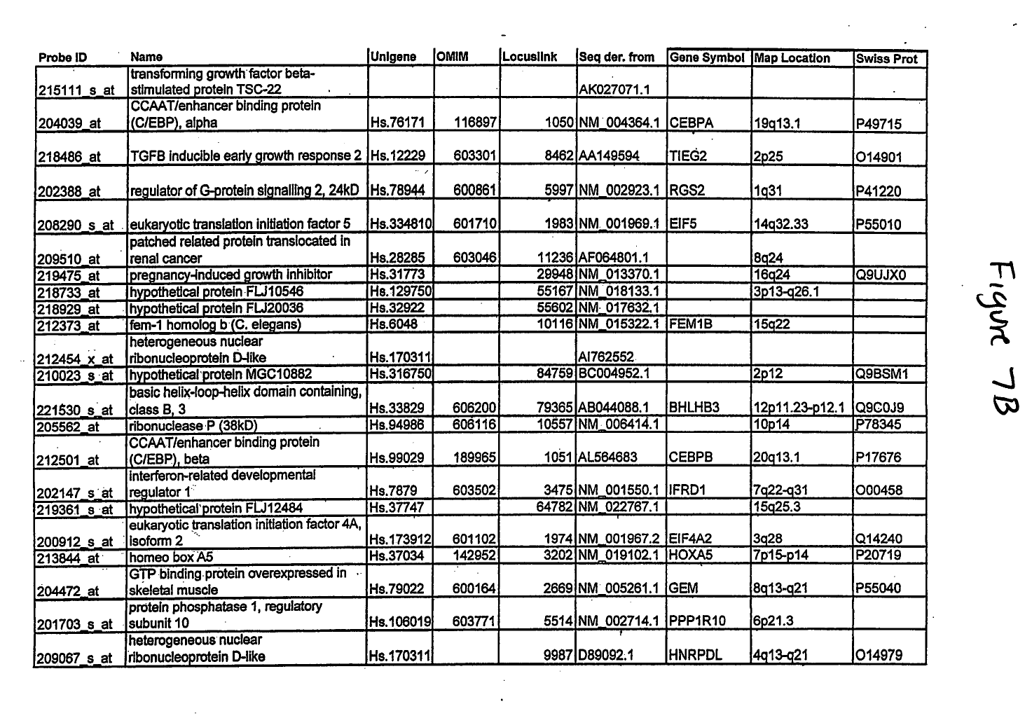

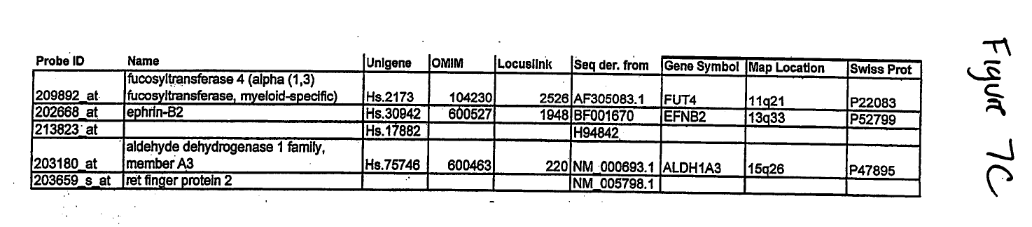

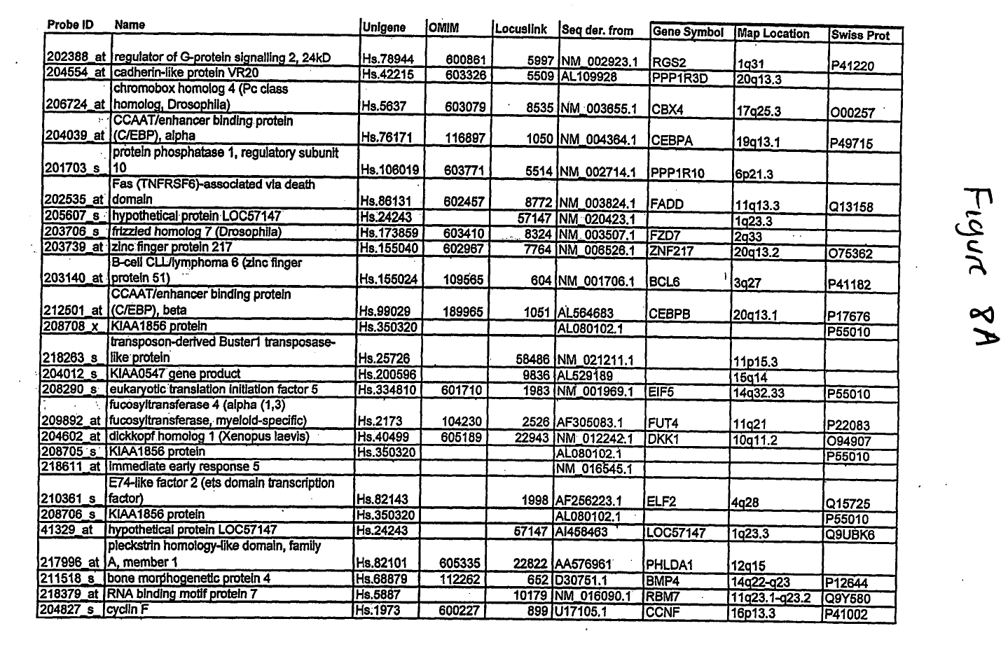

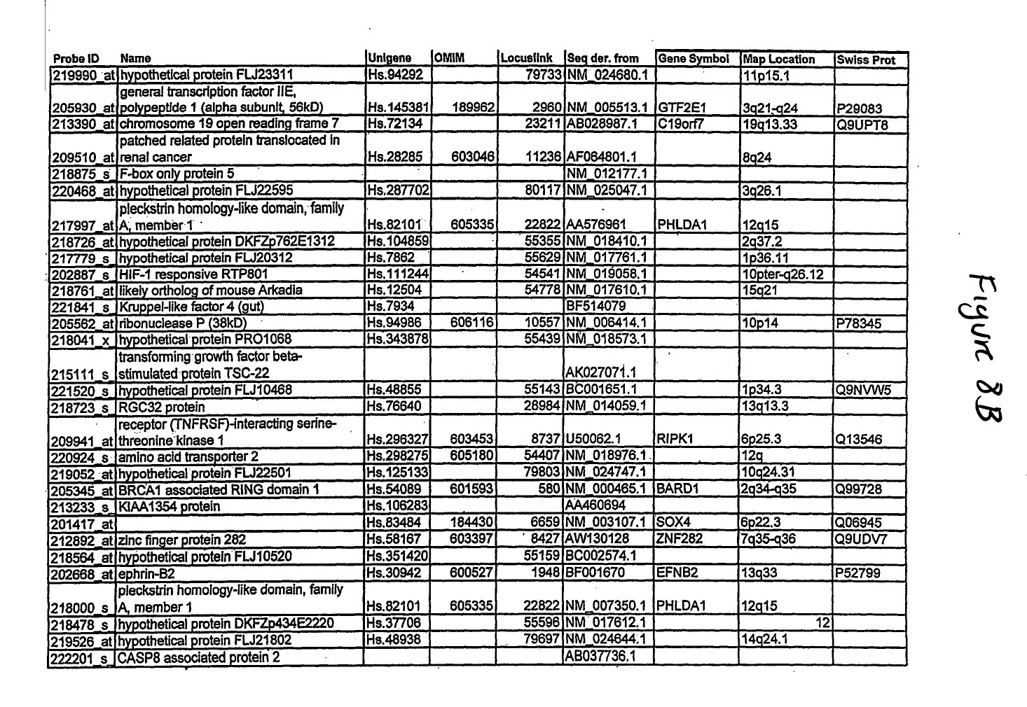

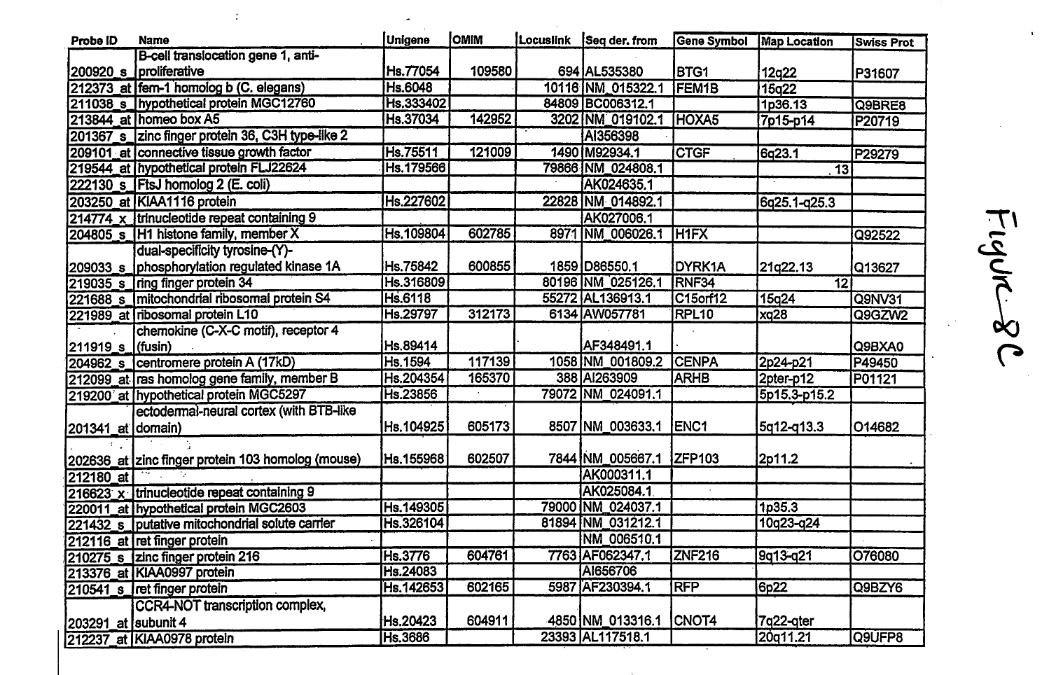

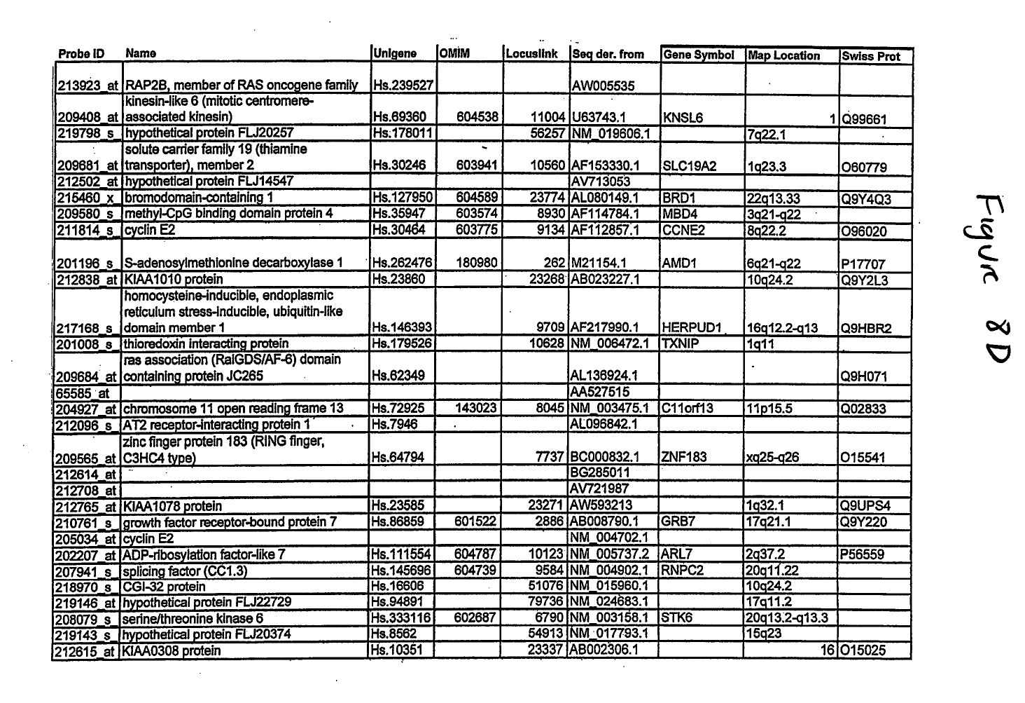

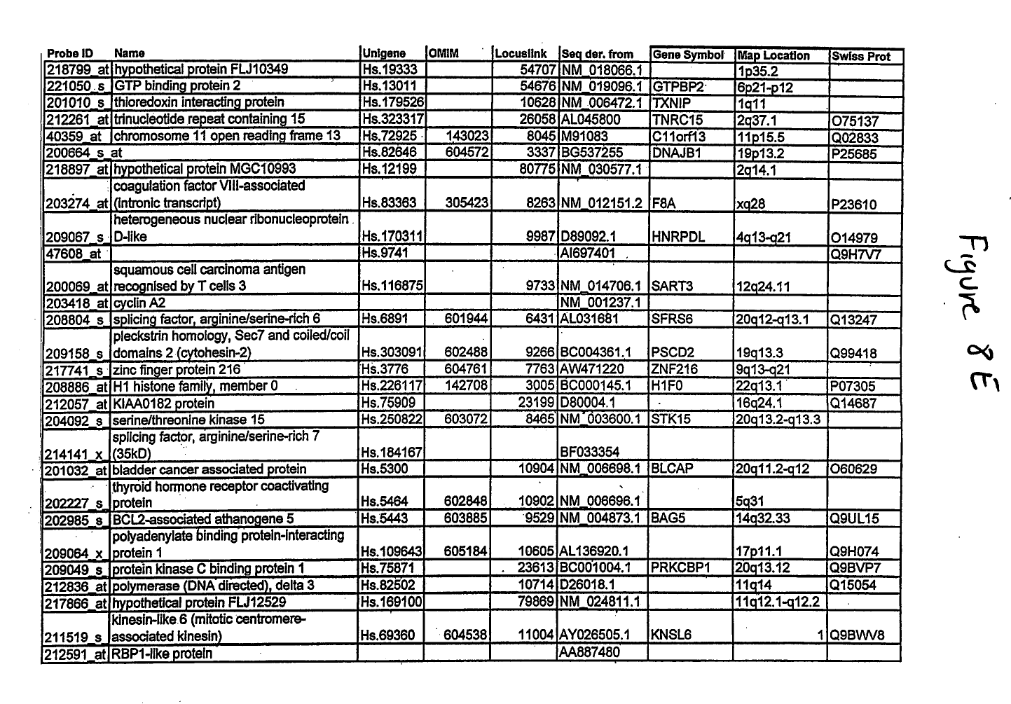

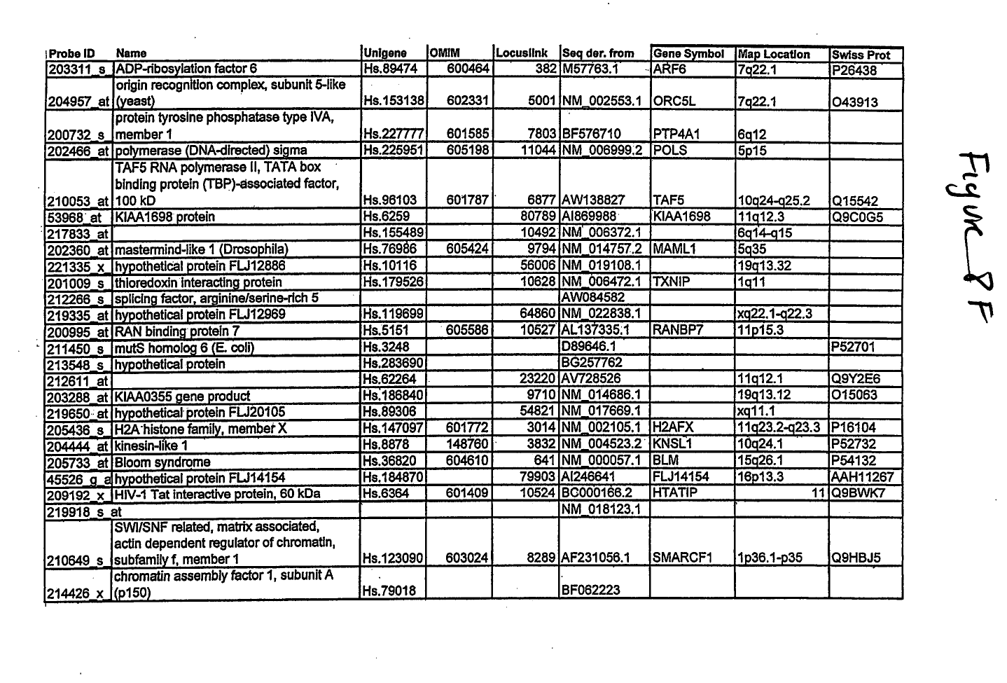

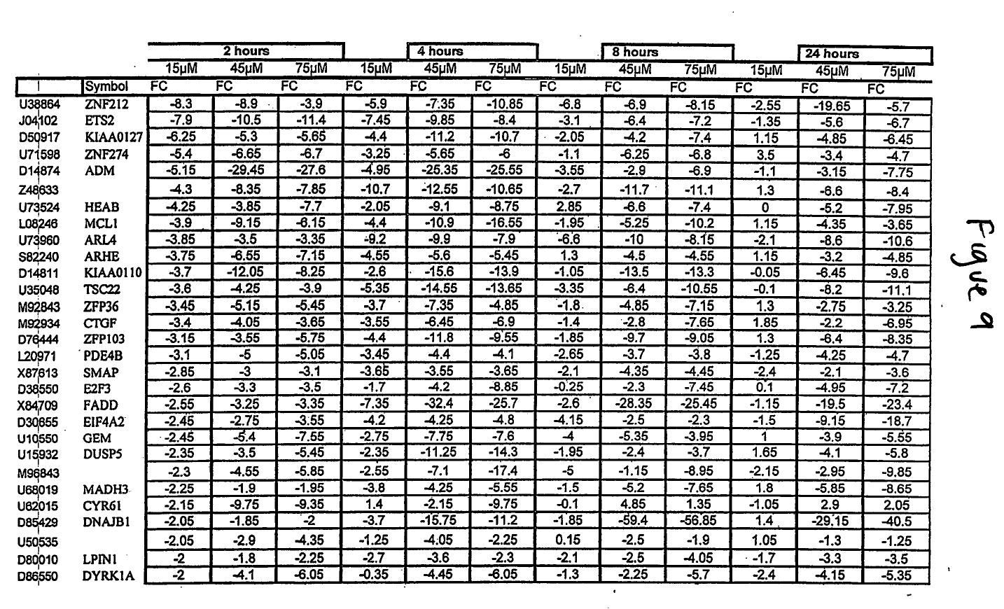

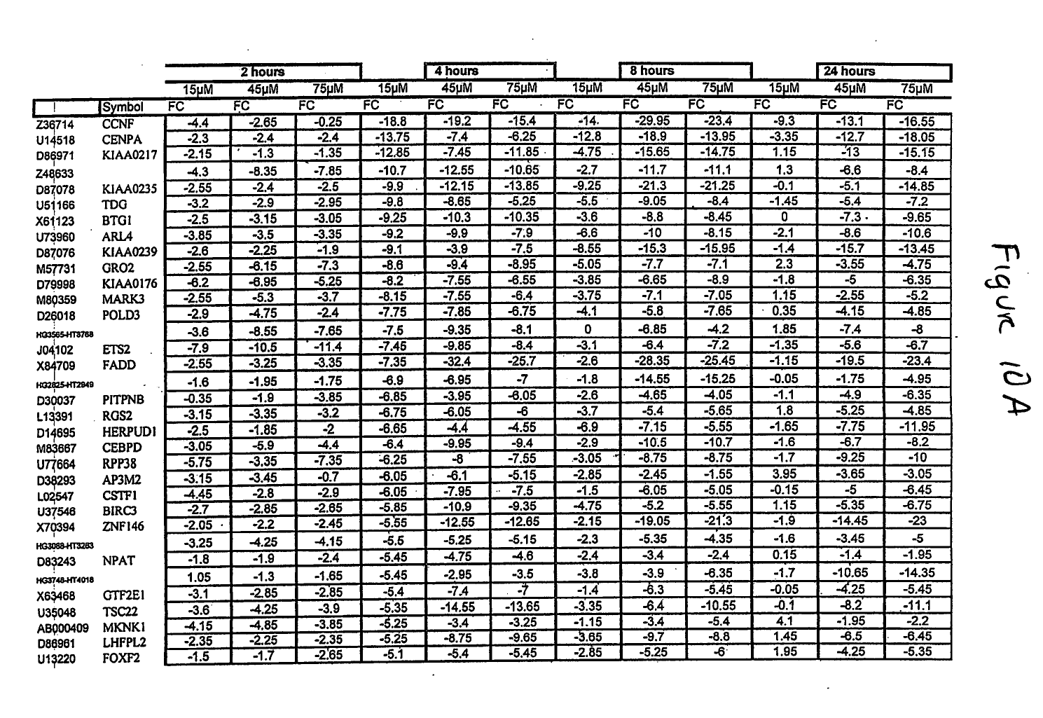

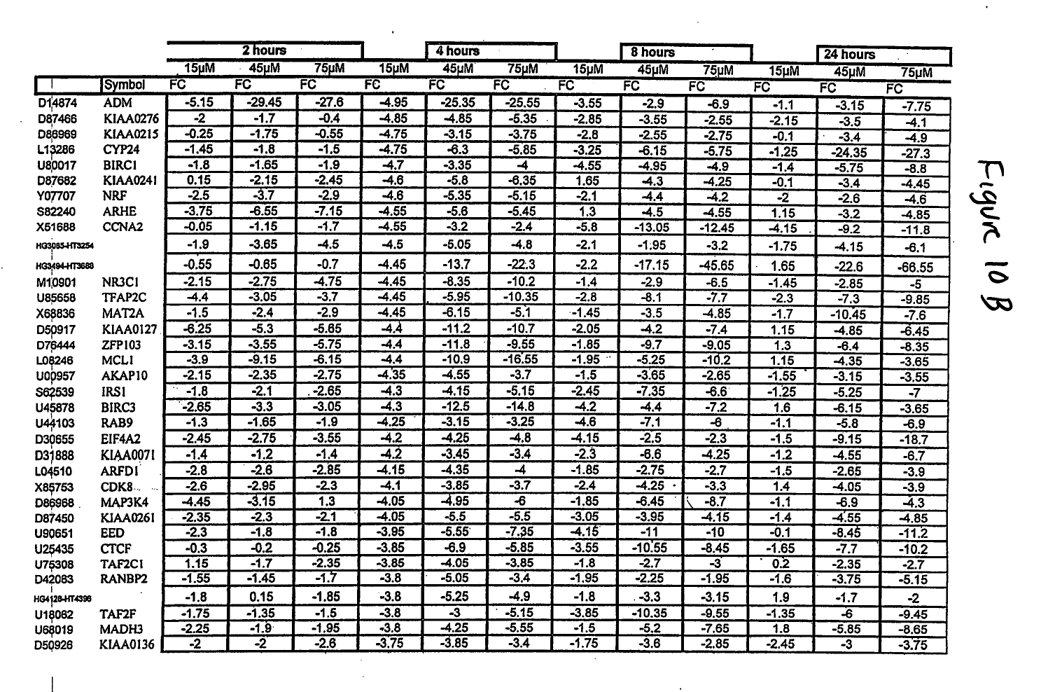

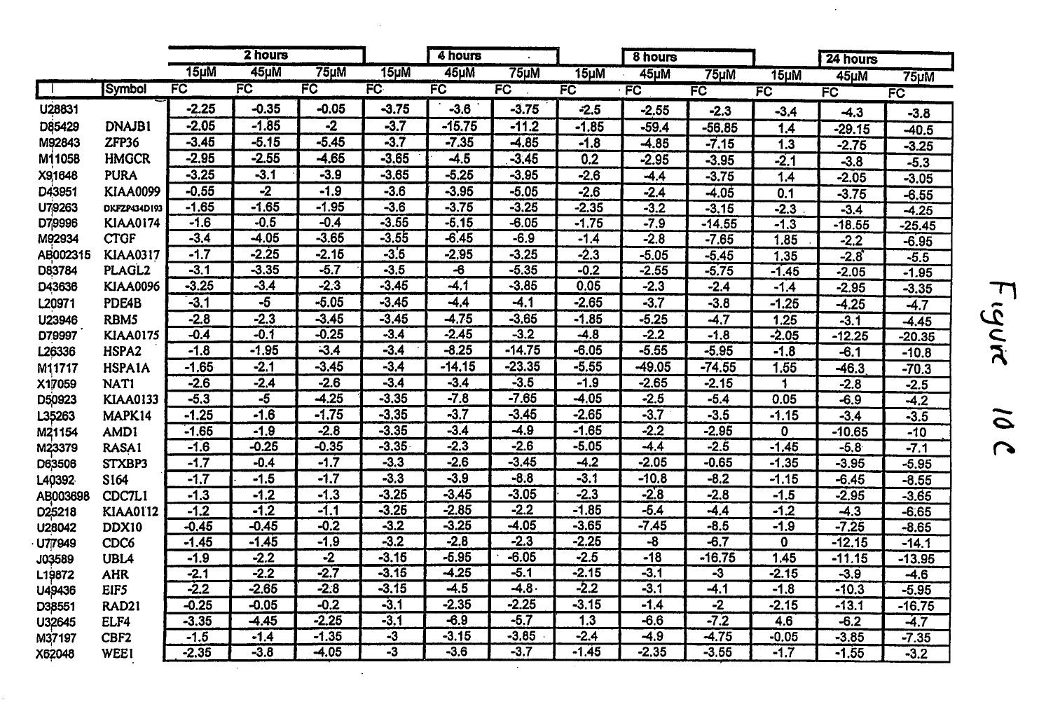

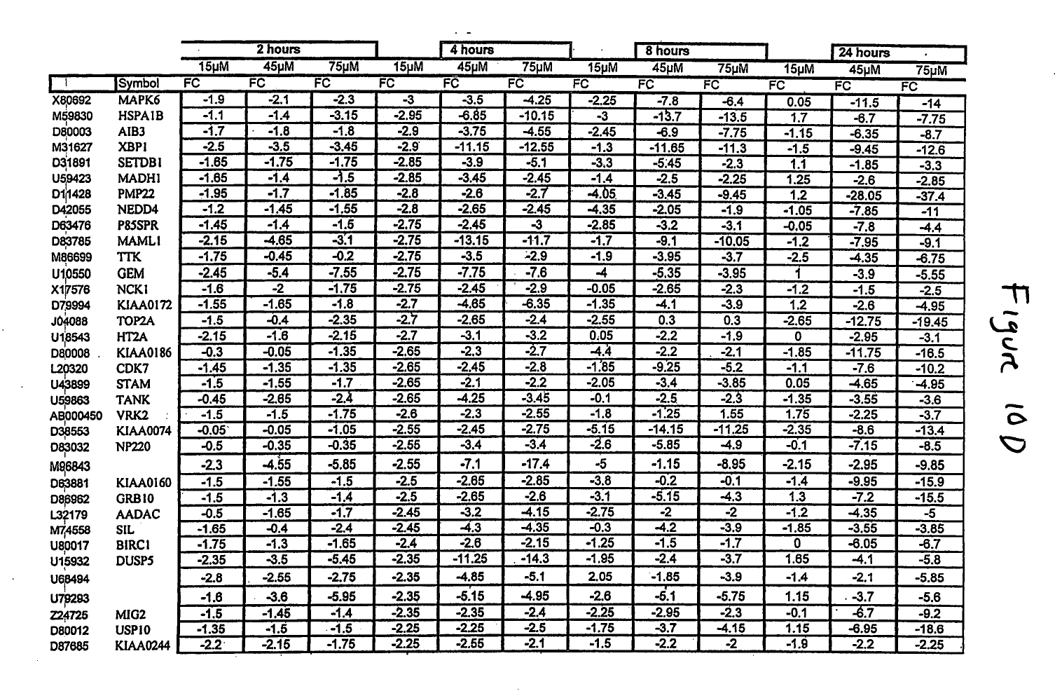

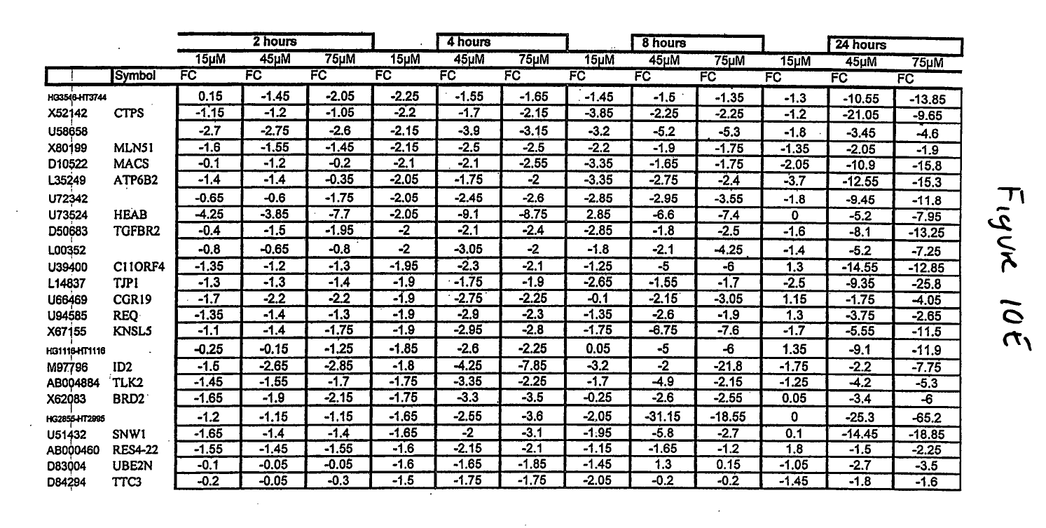

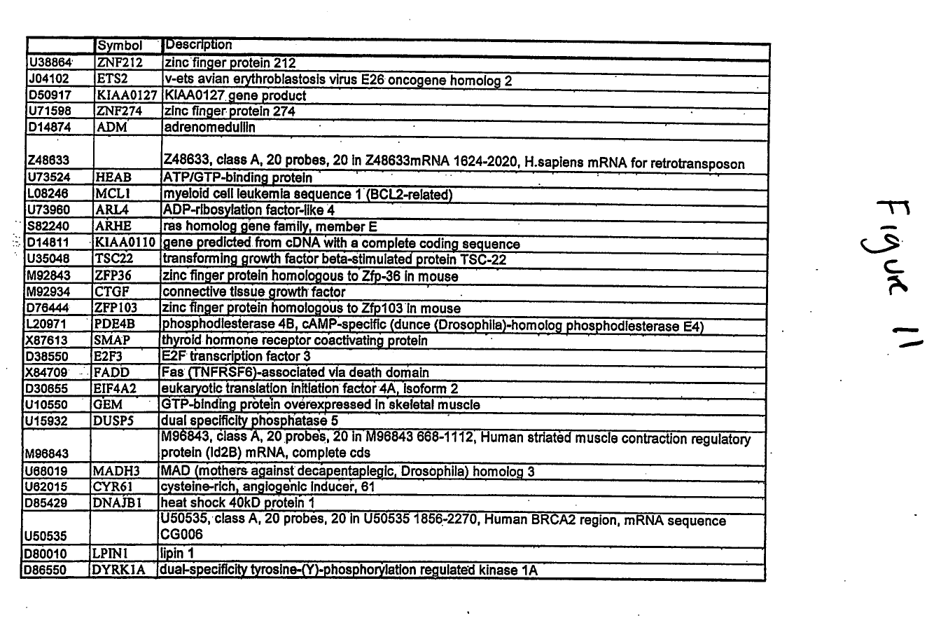

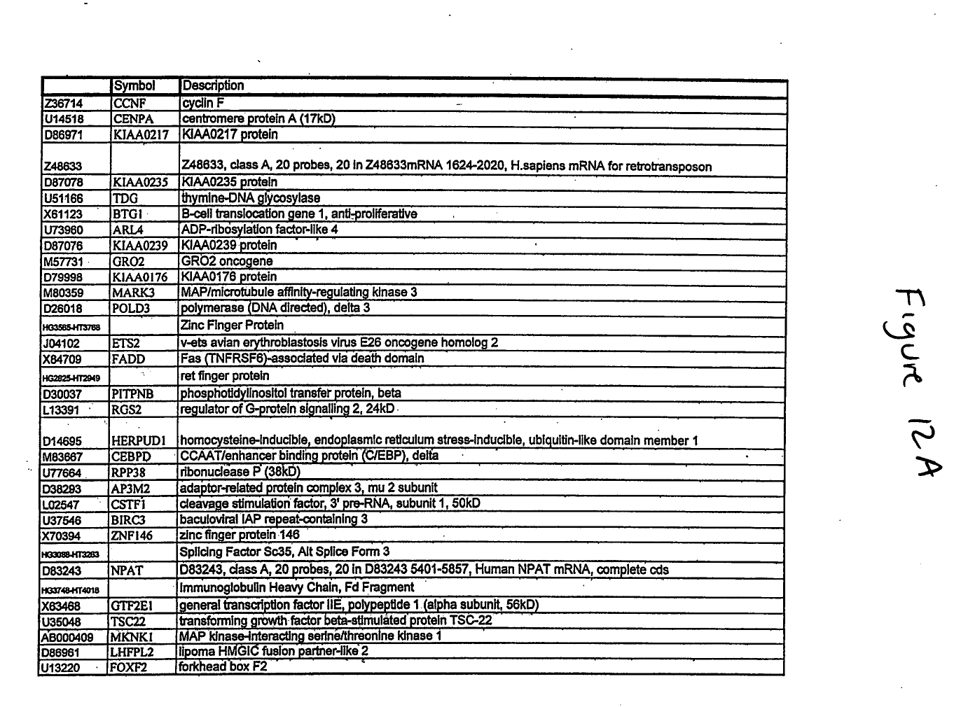

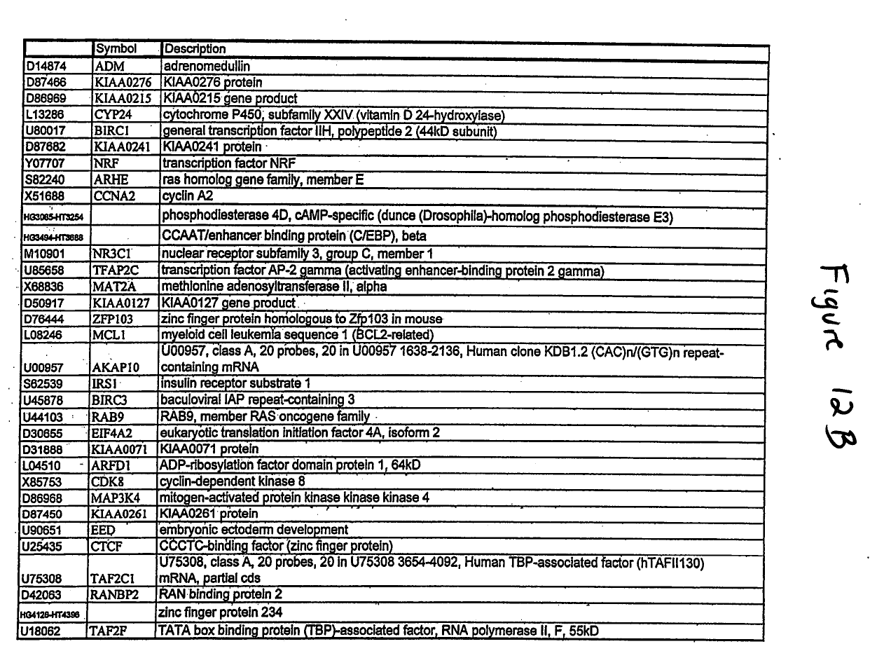

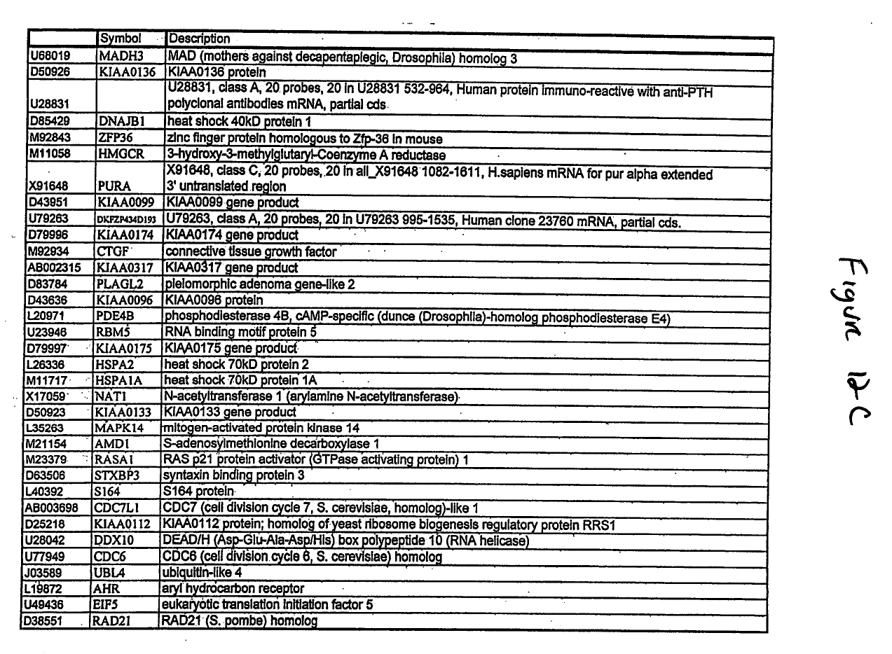

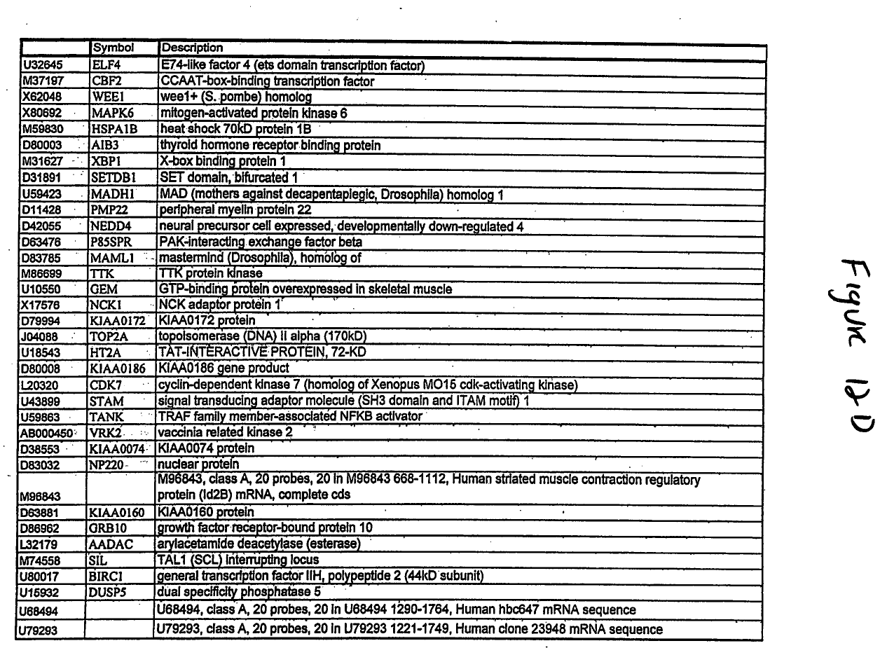

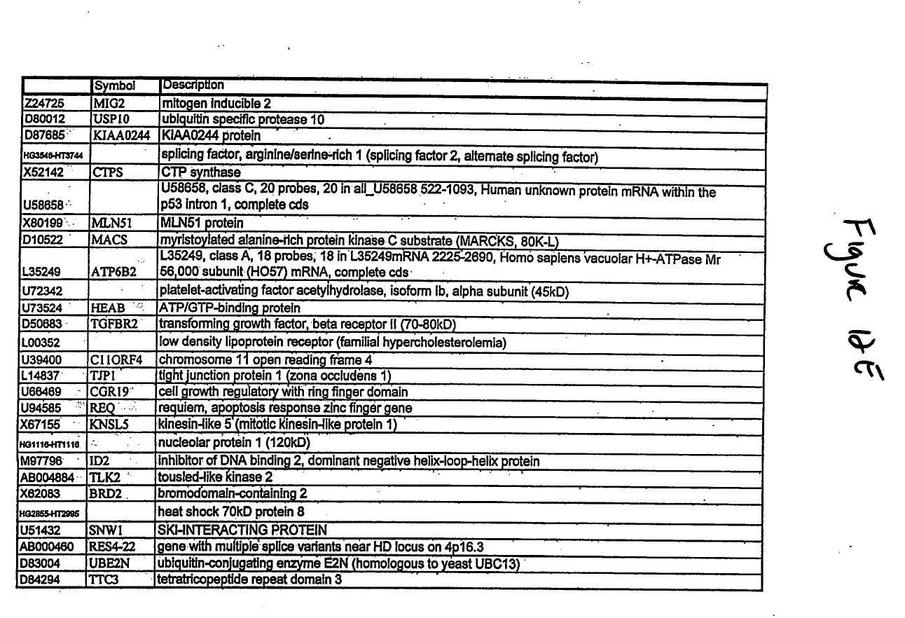

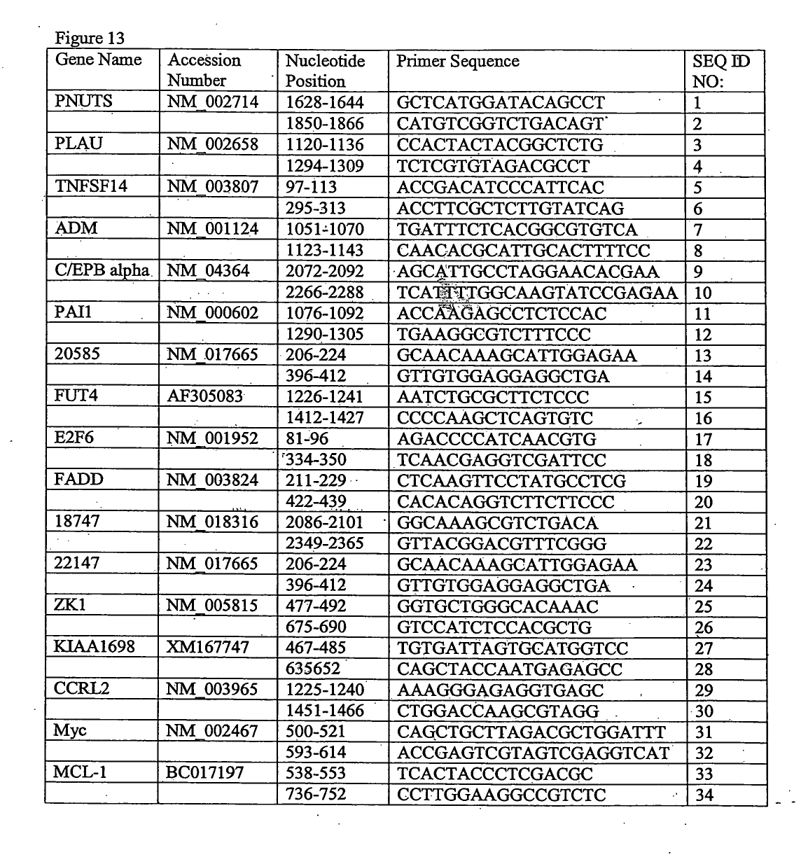

Primers are listed in FIG. 13.

For one-step reverse transcription, the RNA Master SYBR Green I kit (Roche, UK) was used. In a 20 μl reaction volume, 10 ng or 1 μg total RNA was added to 7.5 μl RNA Master SYBR Green I, 3.25 mM Mn(OAc)₂ and 0.3 μM each primer. Reaction conditions were as follows: an RT step for 20 minutes at 61° C., followed by a denaturation step for 2 minutes at 95° C., an amplification step consisting of 45 cycles of 95° C. for 5 seconds, 55° C. for 5 seconds and 72° C. for 13 seconds, followed by a melting curve analysis step to distinguish between primer-dimers and product, comprising of 95° C. for 5 seconds, 65° C. for 15 seconds and increasing to 95° C. at the rate of 0.1° C./second, and finally finishing with a cooling step of 40° C. for 30 seconds.

Analysis of QPCR Data

All samples were normalized to 1 μg/μl, and equivalent total amounts of RNA were used in the assay (either 100 ng or 1 μg, depending on the target gene of interest). Samples were prepared in duplicate and PCR reactions were also run in duplicate. The formula for calculating the fold change in expression levels in the presence of compound compared with the DMSO vehicle control was calculated as follows:
2^{ΔCt}
where 2 is the maximum efficiency of each PCR reaction and ΔCt is the change in crossing point values (sample Ct−DMSO control Ct)

In addition, where the expression levels of housekeeper genes have been measured in these samples, the data has been normalized using a derivative of this formula:
2^{ΔCt}target/2^{ΔCt}housekeeper
Results

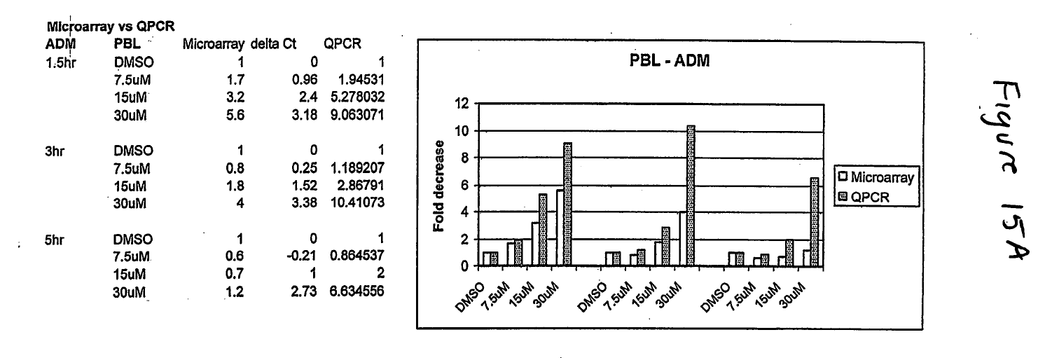

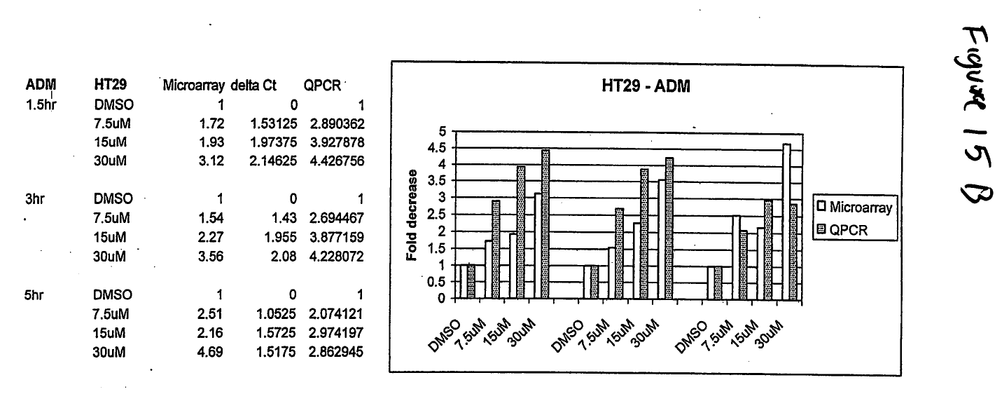

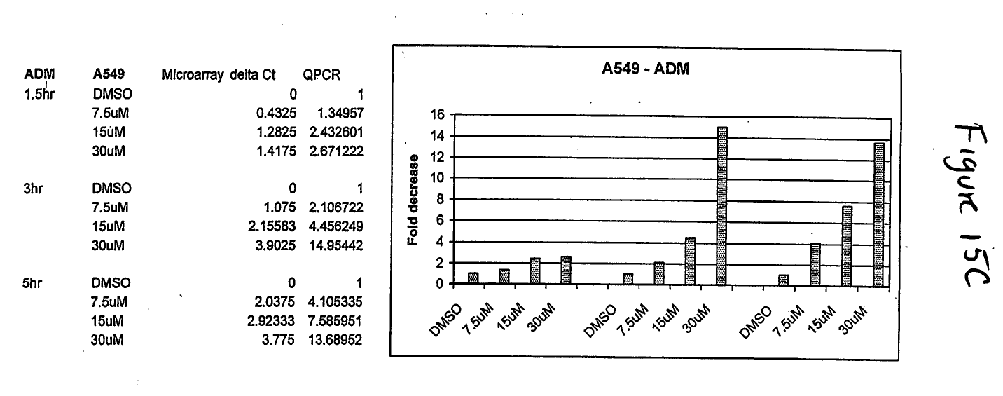

FIGS. 15 to 31 show a graphical representation of a selection of 16 genes (ADM, FADD, PAI1, PLAU, PNUTS, TNFSF14, C/EBP alpha, 20585, FUT4, E2F6, 18747, 22147, ZK1, KIAA1698, CCRL2, myc and mcl-1) whose expression data is presented in the microarray data in FIGS. 1 to 12. The microarray data obtained for each of the 16 genes in PBMC and/or HT29 and/or A549 cells is compared to the results of QPCR analysis.

These results confirm the microarray data.

### Example 3

### Analysis of Gene Expression in a Patient Treated with CYC202

For analysis of patient samples, a method that preserves the RNA expression profile during and immediately after blood is drawn is essential for accurate analysis of gene expression in human whole blood by techniques such as QPCR. PreAnalytix have shown that the copy numbers of individual mRNA species in whole blood can change more than 1000-fold during storage or transport at room temperature. This is caused by rapid degradation of RNA as well as by induced expression of certain genes after the blood is drawn.

Accordingly, the PAXgene (PreAnalytix, Switzerland) method is used for collection, stabilisation and transportation of whole blood specimens, together with the rapid and efficient protocol for isolation of cellular RNA. Use of this system combats the problems relating to erroneous fluctuations in gene expression, and yields several micrograms of high quality RNA from only 2.5 ml whole blood.

Methodology

Blood from volunteers was collected into Vacutainer Heparin CPT tubes at Hawkhill Medical Centre, Dundee and treated as described above in Example 2. After transfer to PAXgene tubes, the tubes were placed at room temperature, 4° C. or −20° C. after an initial incubation of 2 hours at room temperature to allow lysis of the cells.

Blood that had been stored for various lengths of time and under various storage conditions was left to thaw at room temperature for approximately 2 hours, and then processed according to the manufacturer's instructions. The optional DNase digestion step was included. RNA samples were quantified and then used directly in QPCR assays.

Results

Yields of RNA varied between 3-5 ug per 2.5 ml blood, and the RNA was of an accepFigure quality. The expression of one target gene (PNUTS) and one housekeeping gene (HPRT) has been measured in all of these samples.

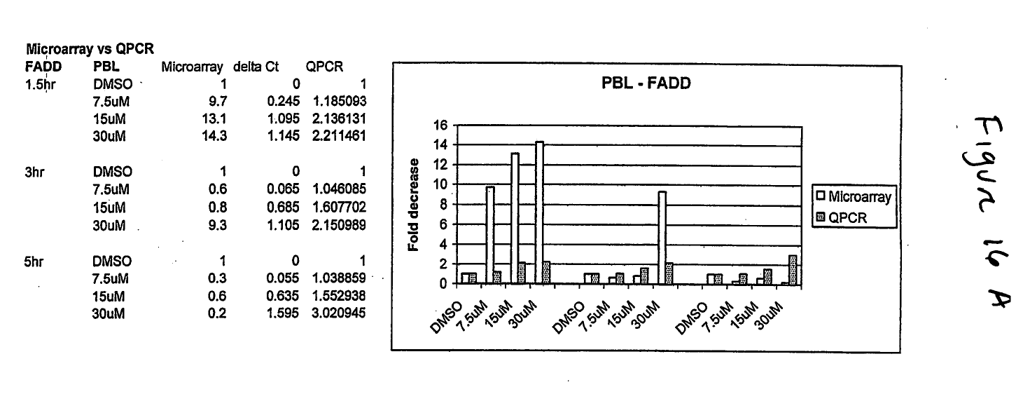

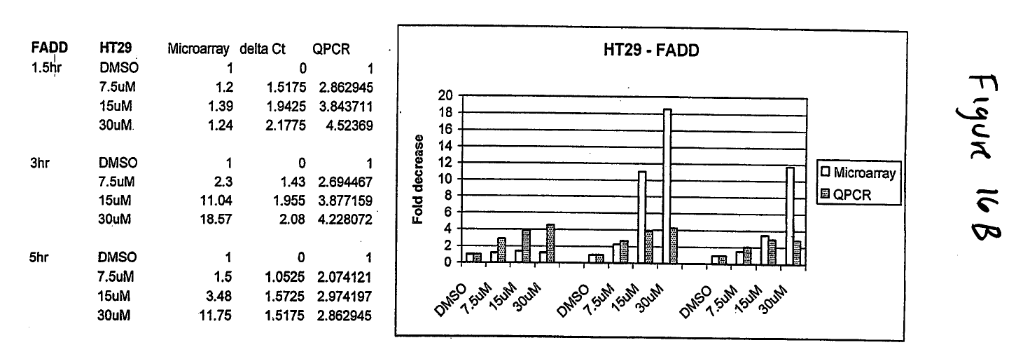

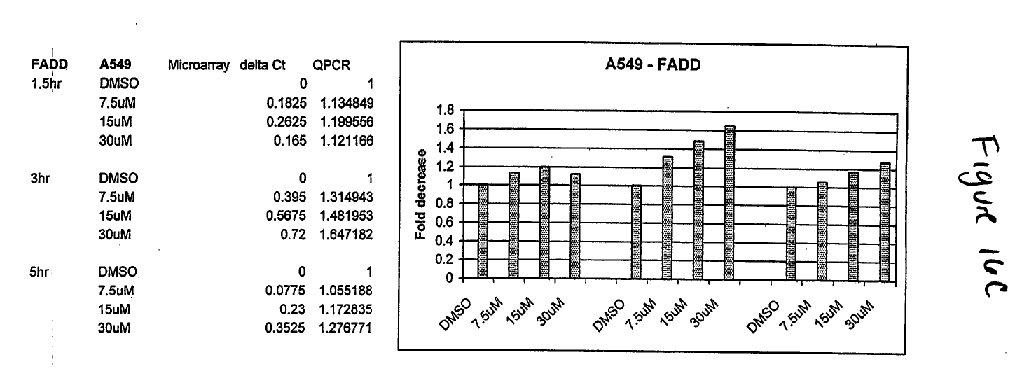

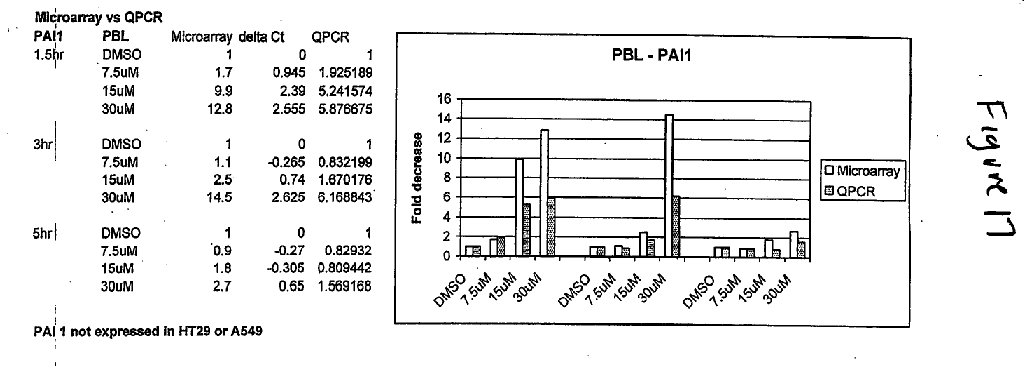

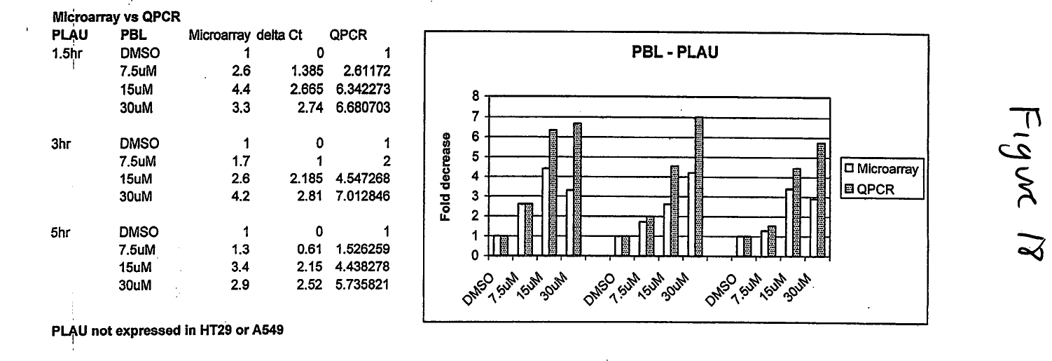

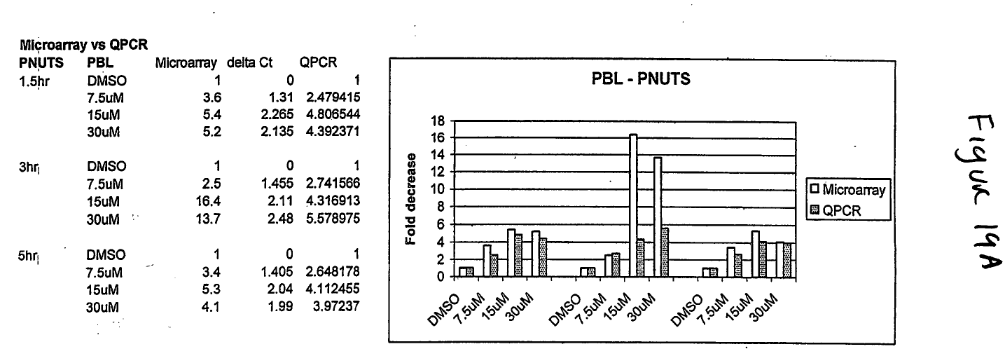

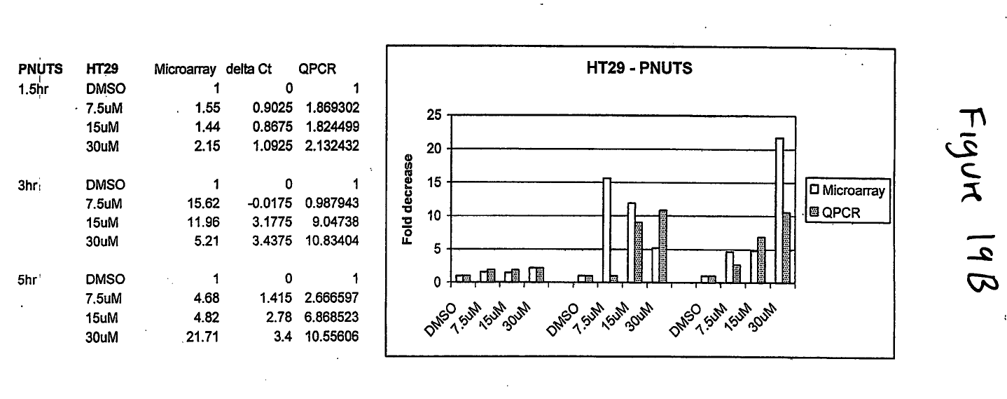

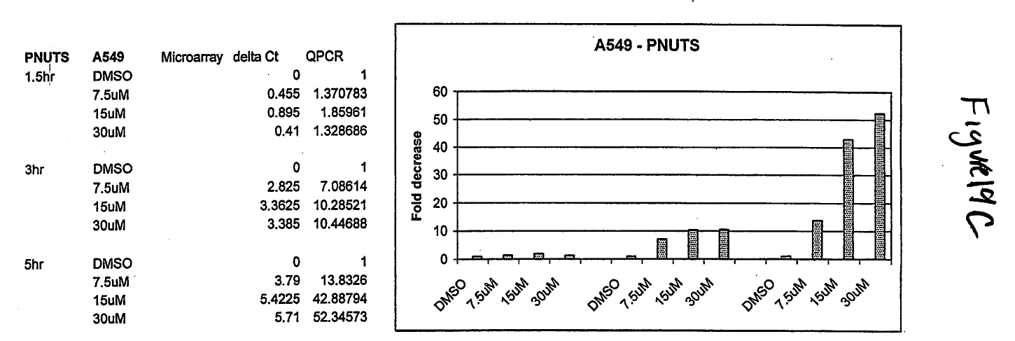

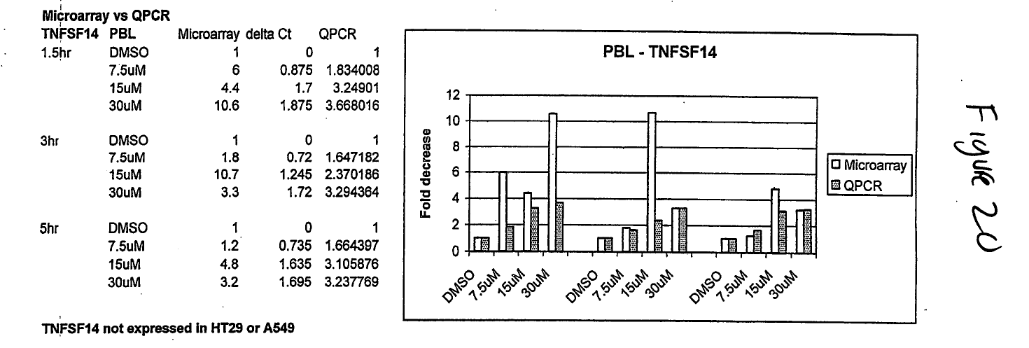

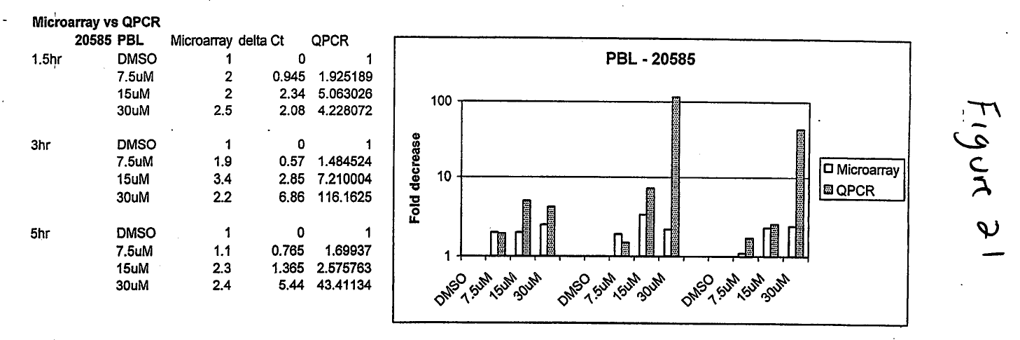

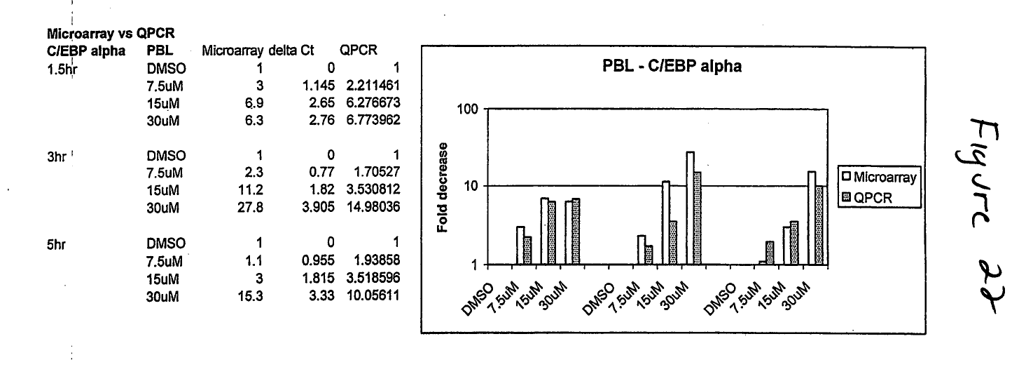

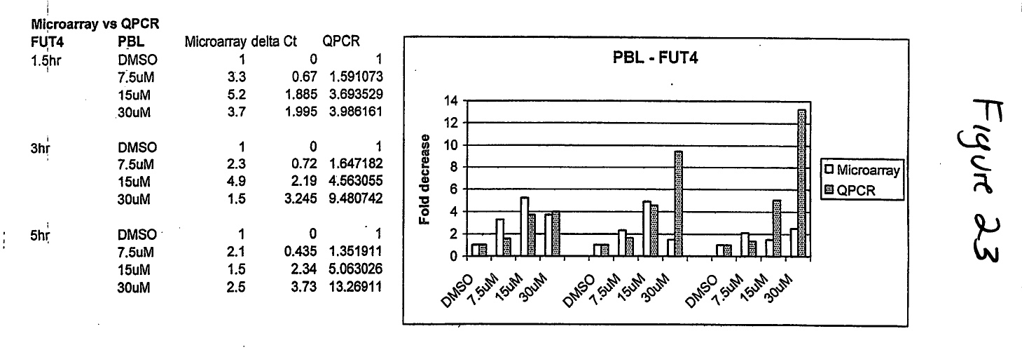

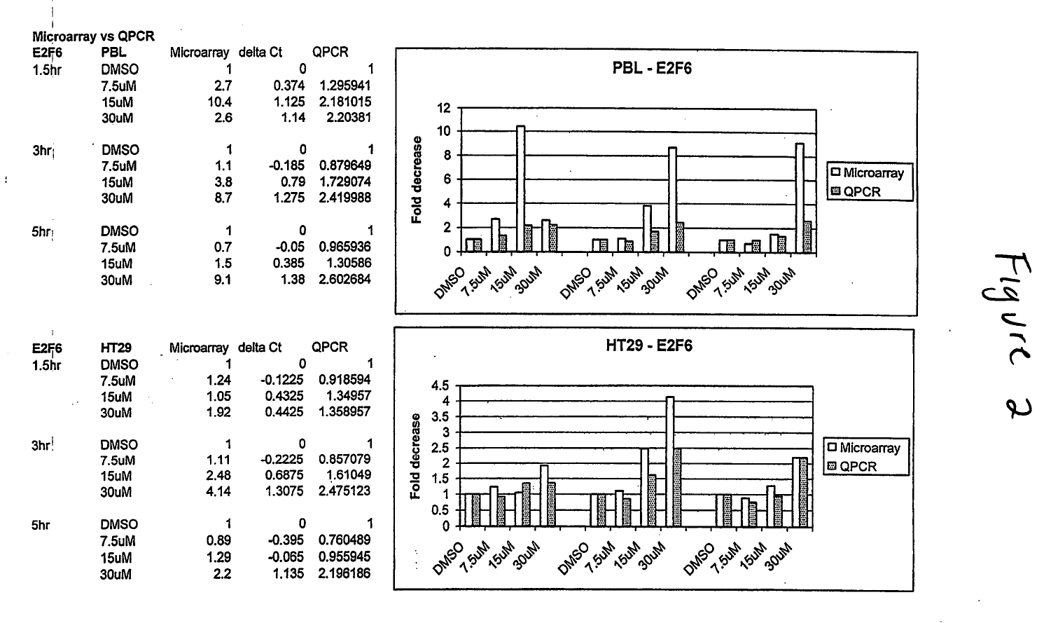

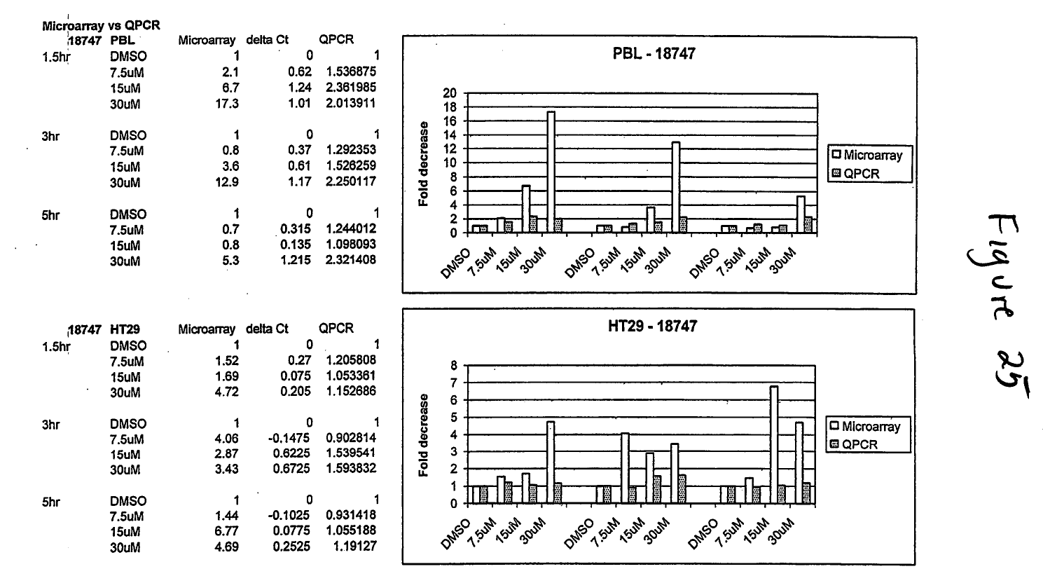

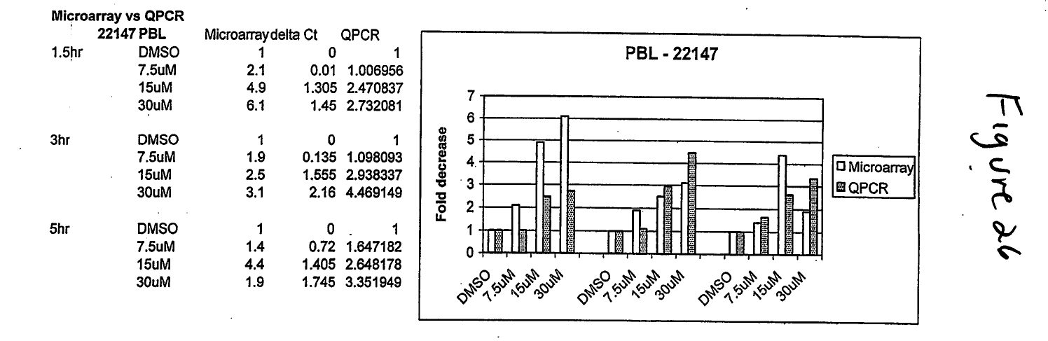

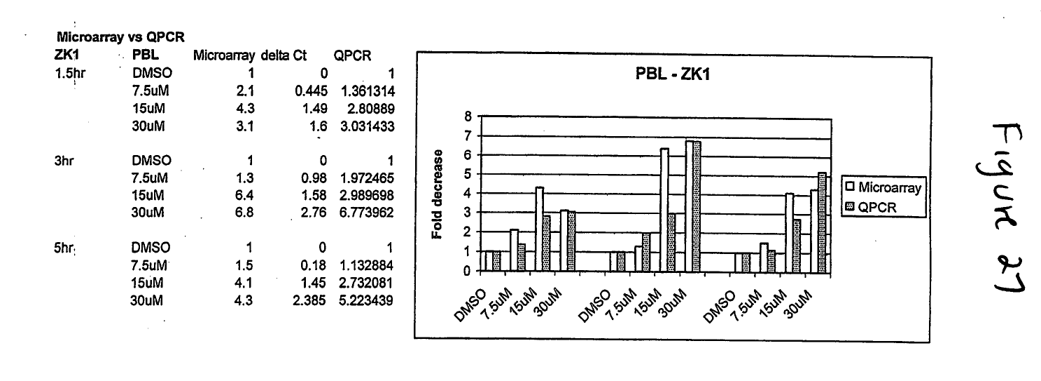

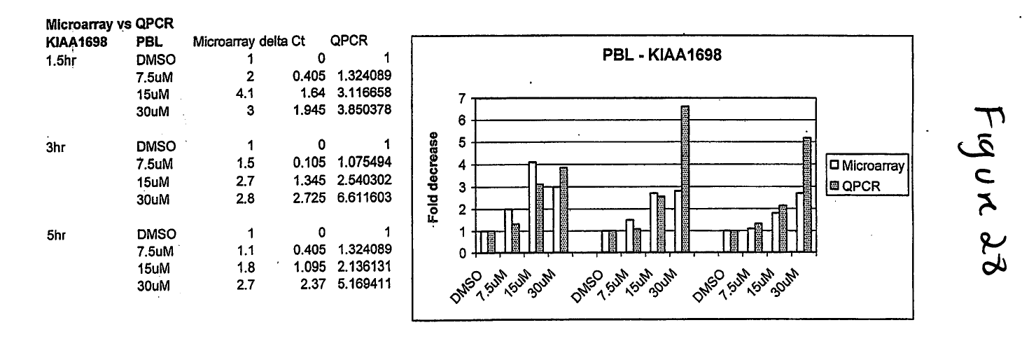

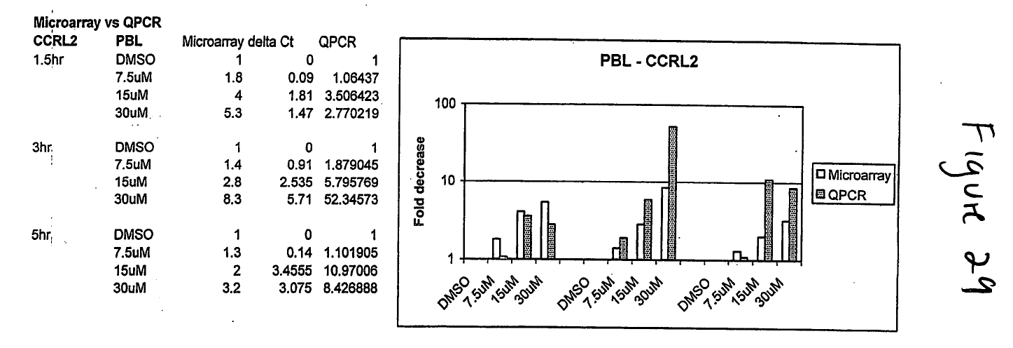

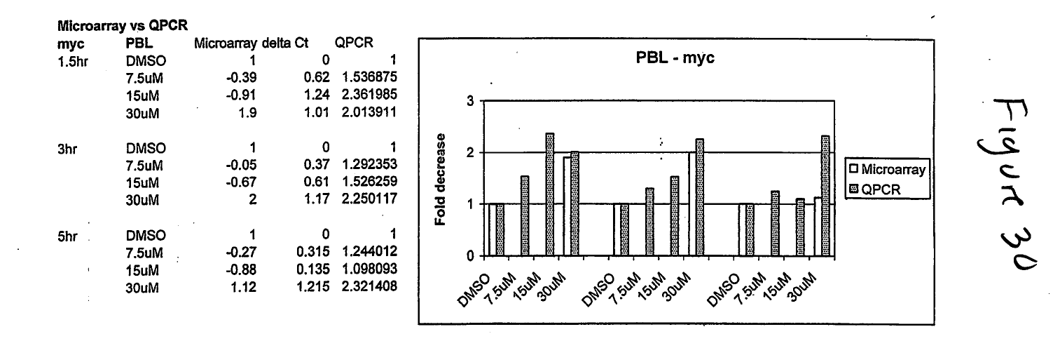

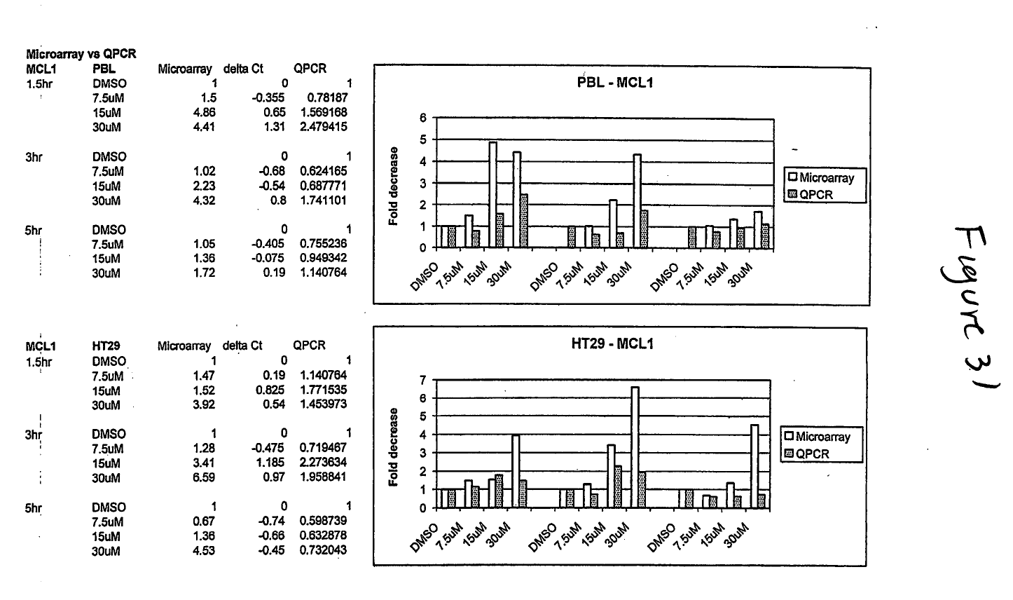

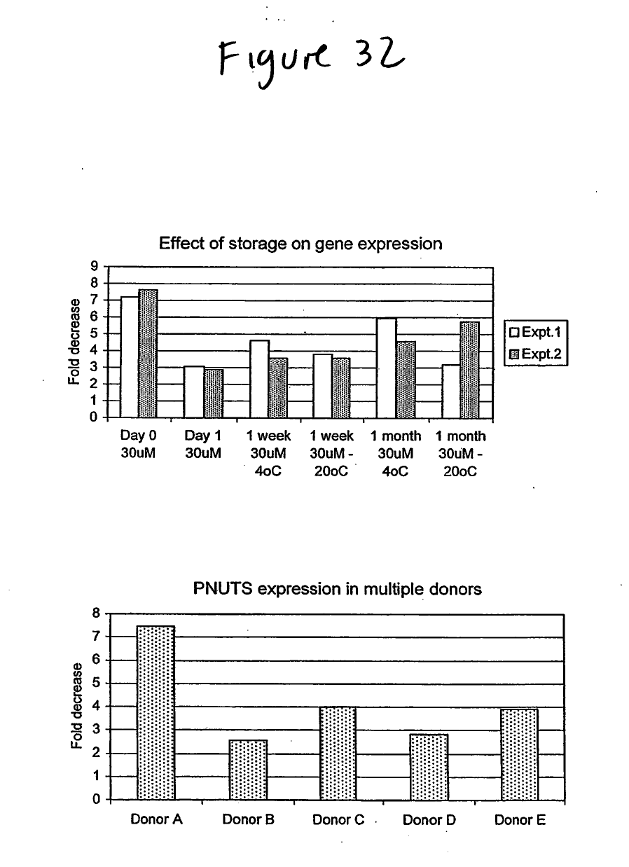

FIG. 32 shows the effect of CYC202 on the expression of PNUTS (lower panel) and the effect of storage on the CYC202-mediated changes in gene expression in a single donor (upper panel). The expression of PNUTS was normalised to that of the housekeeper, and the data in the graph is expressed as the normalised fold decrease in expression of PNUTS following exposure of the blood in vitro to 30 μM CYC202 for a period of 1.5 hours.

Expression of these genes could be detected in all samples, and was reproducibly down-regulated by exposure of the blood to CYC202 (FIG. 32, lower panel). The optimum conditions have been found to be storage at −20° C., and expression has been deemed to be sFigure for up to one month at −20° C. (FIG. 32, upper panel). This is in preference to storage at 4° C. or room temperature, and is in agreement with the manufacturer's recommendations.

Optimisation of QPCR Assays

Optimisation of QPCR assays was undertaken to maximise the differences between the positive and negative signals in order to permit accurate predictions about fold changes in gene expression.

Optimisation was performed using the RNA Amplification kit, and the primer concentration, annealing temperature and MgCl₂ concentration were all varied to obtain the best separation between signal and noise.

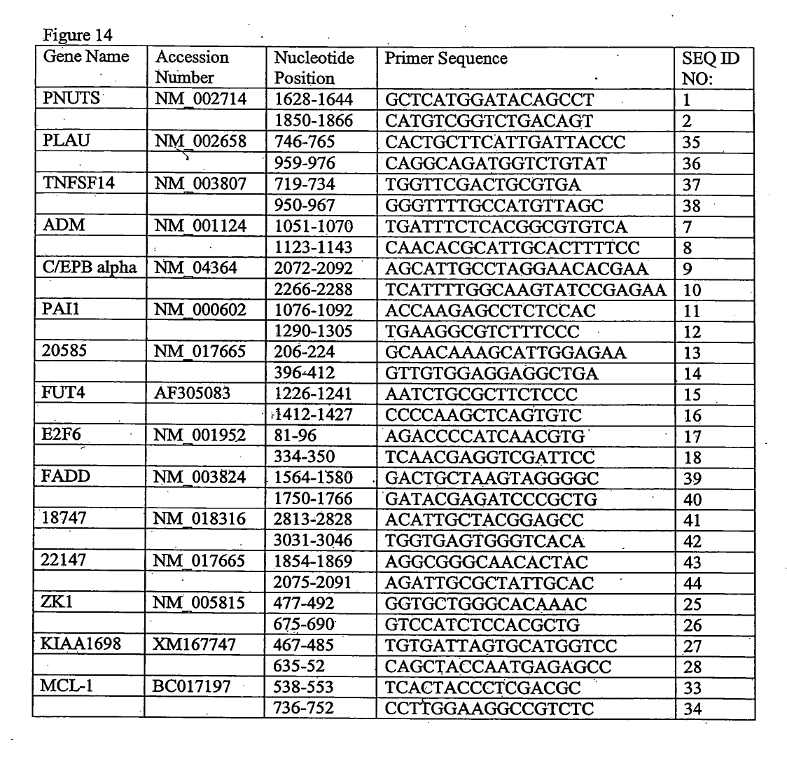

Primers were optimised and the final primer set list is shown in FIG. 14.

In addition, all PCR products were cloned and sequenced to verify that the correct RNA was being amplified.

Analysis of Gene Expression in a Breast Cancer Patient Treated with CYC202

Patient 02-2-01 (08) was treated with 600 mg b.i.d. CYC202 on days 1 to 5, overlapping with capecitabine administration, also oral b.i.d. on days 2-15. Pharmacokinetic analysis was performed on this patient, which revealed that this patient had maintained efficacious levels of CYC202 over several hours.

FIG. 33 shows pharmacokinetic data for patient 02-2-01 (08) showing the data for the full time-course on Day 1 and a single point prior to dose on Day 5. Plasma concentrations of CYC202 are given in μM. Effects on PNUTS and other target genes in vitro has been observed with concentrations of 7.5-15 μM for 1.5-3 hours, which is within the range represented here.

For this reason, it was deemed of interest to analyse the effect of the compound on gene expression in RNA extracted from whole blood taken from this patient at multiple time-points on day 1 of treatment. The expression of PNUTS was examined in all samples and this data was normalised against the expression levels of 28S rRNA which ought to be sFigure.

FIG. 34 is a graph showing the fold decrease in expression of PNUTS, after normalisation with 28S rRNA levels. PNUTS expression is decreased approximately 2-3-fold following the first dose of CYC202, and then returns to normal levels as measured prior to dosing on Day 5.

FIG. 35 is a graph showing the fold decrease in expression of C/EBPalpha, after normalisation with 28S rRNA levels. The decrease in C/EBPalpha expression peaks at 8 hr after the first dose of CYC202 by approximately 4.5-fold and then returns to normal levels as measured prior to dosing on Day 5.

FIG. 36 is a graph showing the fold decrease in expression of FUT4, after normalisation with 28S rRNA levels. The decrease in FUT4 expression peaks at 3 hr after the first dose of CYC202 by approximately 2-fold and then returns to normal levels by 8 hrs after the first dose and as measured prior to dosing on Day 5.

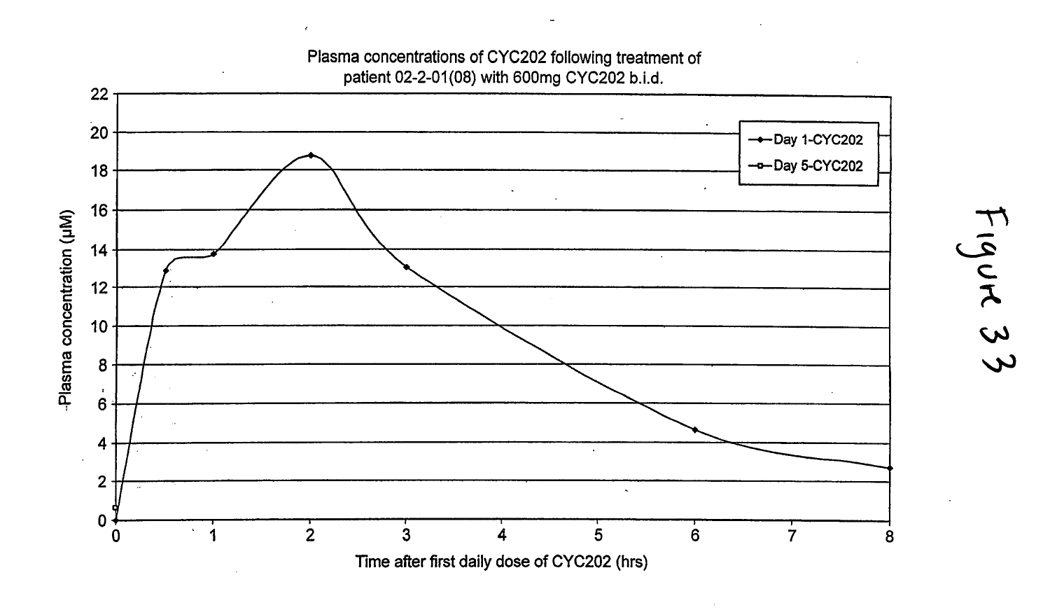

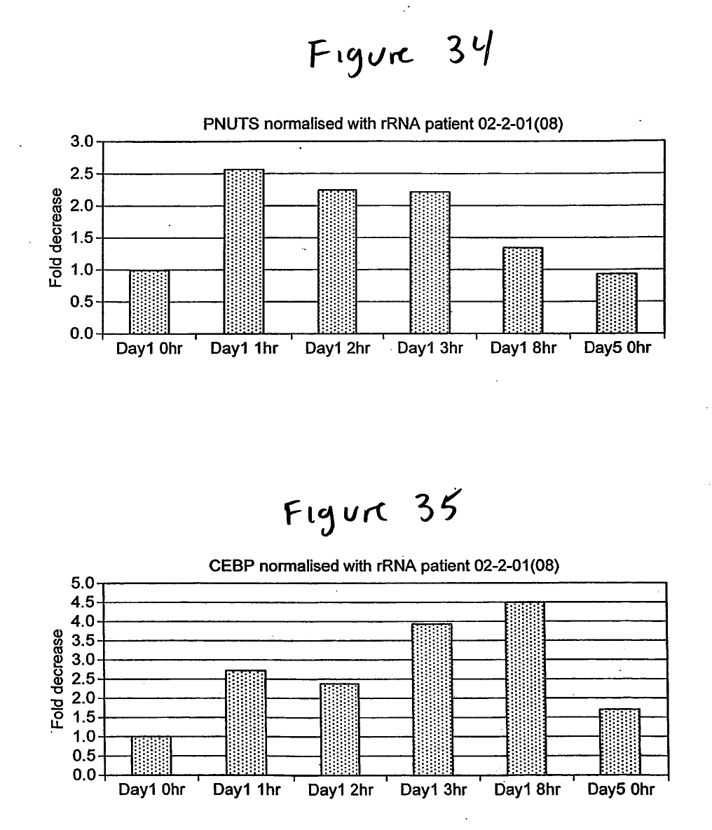

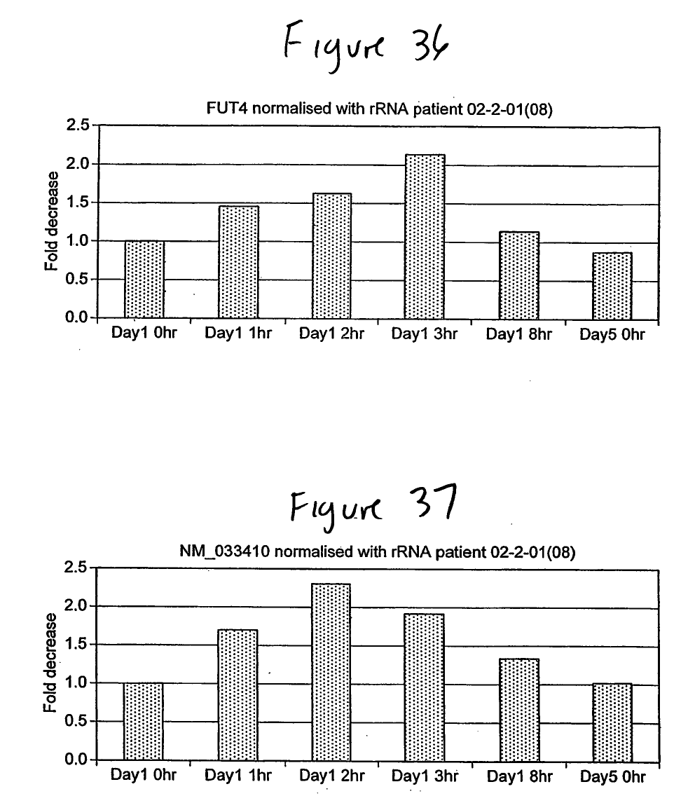

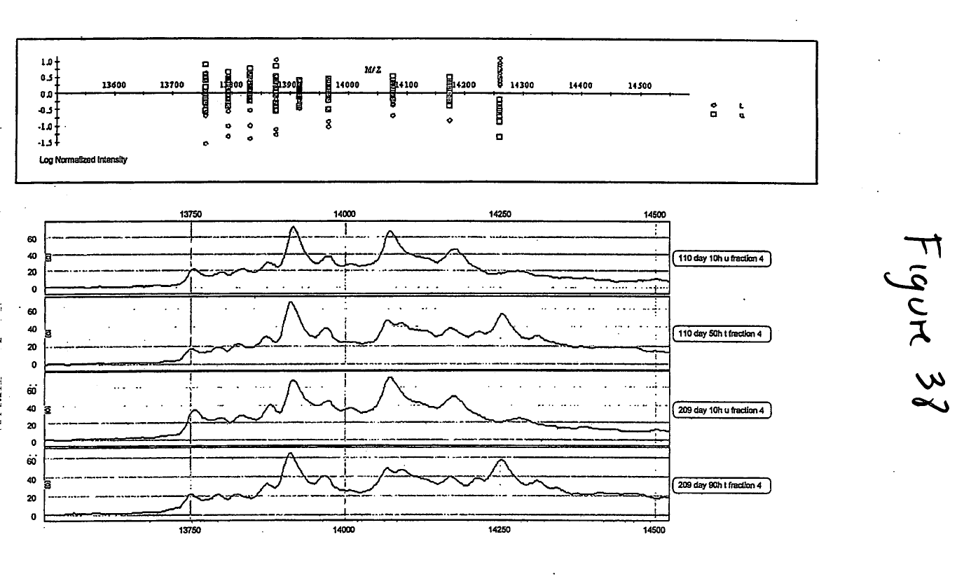

FIG. 37 is a graph showing the fold decrease in expression of NM_{—}033410, after normalisation with 28S rRNA levels. The decrease in NM 033410 expression peaks at 2 hr after the first dose of CYC202 by approximately 2-fold and then returns to normal levels as measured prior to dosing on Day 5.

Conclusions

The kinetics of the effects on gene expression vary considerably with the maximum effects on gene expression occurring at different times after the dose of CYC202 for different genes. In the case of PNUTS, the changes in gene expression mimic the PK data very closely. All RNA samples were normalised to 1 mg/ml and following dilution, were measured for concentration again to ensure all samples were at the same concentration. Moreover, this was confirmed by analysing the expression of 28S rRNA. Any small fluctuations in 28S rRNA would take into account any small variations in RNA concentration between samples, since the expression of all genes was normalised to 28S rRNA.

This data confirms that the differential effects on gene expression seen here are not due to differences in RNA concentration but are real changes that occur in patients treated with CYC202 in the expression of several genes highlighted by the in vitro microarray and QPCR experiments on peripheral blood mononuclear cells and tumour cells.

### Example 4

### Analysis of Plasma Proteomic Profiles and Identification of Biomarkers Using SELDI-TOF-MS

SELDI

Plasma samples were obtained from patients on the first and last days of treatment with CYC202. A total of 16 patients were analysed in this study, consisting of patients with different tumour types, and receiving different doses and scheduling regimes of CYC202. 10 patients were on 5 days continuous treatment from 1.6 g to 3.2 g CYC202 per day every 3 weeks, 4 patients were on 10 days continuous treatment every 3 weeks from 1.6 g to 2 g CYC202 per day, 2 patients were on 3 days treatment every 3 weeks 2.4 g CYC202 per day. All samples were stored at −80° C. in aliquots.

Samples were analysed on 4 different ProteinChip® Array surfaces either neat or following fractionation on a Q Ceramic HyperD®F 96 well plate (Ciphergen). The technique separates proteins from a complex biological source into fractions on the basis of charge. Anion exchange sorbents are designed for fractionation of proteins such that proteins having similar pI or binding affinity to the ion exchangers elute together. This also provides the benefit that highly abundant proteins in the sample are segregated into a limited number of fractions, reducing their signal suppression effects on lower abundance proteins, as well as ensuring that the capacity of the ProteinChip® Array surfaces are not exceeded.

For fractionation, 20 μl of each plasma sample was mixed with 30 μl U9 buffer (9M urea, 2% CHAPS, 50 mM Tris-HCl pH9) for 30 minutes at room temperature. It was then diluted with the same volume of U1 buffer (1M urea, 0.2% CHAPS, 50 mM Tris-HCl, pH9) prior to addition to the Q HyperD® F plate. The fractionation procedure was completed as per the manufacturer's instructions for the Expression Difference Mapping™ kit, which basically involved elution by successive lowering of pH to yield 6 fractions.

All chip surfaces were equilibrated with the appropriate binding buffer as detailed below. Fractionated samples were then applied to the various surfaces at a 1:10 dilution with the appropriate binding buffer. Neat samples were diluted 1:6 in U9 buffer, mixed for 30 minutes at room temperature and then also diluted 1:10 with the appropriate buffer for binding to the ProteinChip® Array surfaces. SAX-2 or Q10 ProteinChip® Arrays are strong anion exchange surfaces, and samples were applied to these ProteinChip® Arrays in pH9 buffer (100 mM Tris-HCl pH9, 0.1% Triton X-100). WCX2 and CM10 chips are weak cation exchange surfaces and the buffer used was 100 mM NaOAc pH3.5, 0.1% Triton X-100. The hydrophobic chip surface (H50) utilised 10% ACN+0.1% TFA and IMAC chips were activated with 0.1M cupric sulfate according to manufacturer's instructions. All samples were allowed to bind for 1 hour at room temperature on a platform shaker. The arrays were washed once with the binding buffer, followed by two washes with the binding buffer in the absence of detergent, each for 5 minutes on the shaking platform. They were then rinsed briefly with 10 mM Hepes pH7 and left to air-dry. 0.8 μl of 50% saturated sinapinic acid (prepared in 50% acetonitrile, 0.05% TFA) was applied twice to each spot. Proteins were then detected with the ProteinChip® Reader. Data was collected using three different mass ranges; from 0-50,000 (low), 0 to 100,000 (mid) and 0 to 200,000 (high) by averaging approx. 150 laser shots with an intensity of between 190 and 210.

Using the Biomarker Wizard Software all spectra were compiled, and qualified mass peaks (signal-to-noise ratio>5) with mass-to-charge ratios (m/z) between 2000 and 200,000 were autodetected. Peak clusters were completed using second-pass peak selection (signal-to-noise ratio>2, within 0.3% mass window), and estimated peaks were added. The peak intensities were normalized to the total ion current of m/z between 2000 and 50,000, 100,000 or 200,000 depending on whether the data was extracted from the low, mid or high mass range. All these were performed using ProteinChip® Software 3.1 (Ciphergen). The only additional preprocessing step was logarithmic transformation of the peak intensity data. The Biomarker Wizard groups peaks of similar molecular weight from across sample groups of spectra, then statistically and visually displays differences in expression levels between sample groups. The mean and standard deviation for each sample group is reported and the appropriate statistics applied. Parametric tests are applied to very large data sets, but for this study, non-parametric tests have been performed, which assume that the data is too small to have a normal distribution. In this case the Mann-Whitney U test is used to analyse the data and a p-value less than 0.05 is assigned to each cluster group that is deemed to be statistically significantly different between the two groups, representing before or after treatment.

2D Gels

2D gel electrophoresis was carried out using the IPGPhor (Amersham Pharmacia Biotech) system for the first-dimensional isoelectric focusing and Novex MiniCell system (Invitrogen) for the second-dimensional SDS-PAGE.

Samples for isoelectric focusing were loaded into Immobiline DryStrips (7 cm, pH3-10 (linear) or pH4-7), by in-gel rehydration using rehydration buffer (8M urea, 2% CHAPS, bromophenol blue and 0.5% IPG buffer appropriate to the pH) plus 511 plasma sample plus 20 mM DTT in the IPGphor at 20° C. for 16 hrs, according to the manufacturer's instructions. Proteins were focused in the first dimension using a total of approximately 40000V over a period of 8 hrs with a constant current of 50 mA per strip. After isoelectric focusing, the Immobiline DryStrips were equilibrated at room temperature for 30 minutes with equilibration buffer containing 50 mM Tris-HCl, pH8.8, 6M urea, 30% glycerol, 2% SDS, 0.01% Bromophenol blue and 10 mg/ml DTT. This was followed by a further 15 minutes in equilibration buffer with no DTT, but containing 25 mg/ml iodoacetamide.

Second-dimensional electrophoresis was performed using 4-12% gradient SDS-PAGE gels. The focused/equilibrated Immobiline DryStrips were placed in direct contact with the SDS-PAGE gels and proteins were separated using electrophoresis at a constant voltage of 150V until bromophenol blue reached the bottom of the gel.

Colloidal Blue staining (Invitrogen) was carried out as per the manufacturer's instructions, and spots of interest were excised from duplicate gels and processed in parallel for protein identification and protein extraction by passive elution.

Protein Identification

For protein identification, gel pieces were incubated sequentially in 100 mM ammonium bicarbonate/50% acetonitrile for three washes of 10 minutes each to remove excess stain and SDS, then with 100% acetonitrile for 5 minutes. This solution was removed and the gel pieces incubated on a heat block briefly to dehydrate the gel pieces. Trypsin (Promega porcine trypsin at 10 ng/ul in 25 mM ammonium bicarbonate) was then added and the gel pieces incubated overnight at 37° C. to digest the protein. To ascertain whether the tryptic digestion has been successful, samples were analysed on the SELDI-TOF MS. To do this, 0.51 μl of the tryptic digest was mixed with 0.5 μl of a 20% solution of a-cyano-4-hydroxycinnamic acid (CHCA) matrix in 50% ACN, 0.5% TFA, and applied to an NP20 chip. To identify the protein, the peptide digests were submitted to the University of Dundee ‘FingerPrints’ Proteomics Facility. The digests were analysed by MS and MS-MS on an ABI 4700 Proteomics Analyzer with Tof/Tof Optics. The combined MS and MS-MS data from the peptide mass fingerprints of each digested gel spot was used to search the CDS database using the Mascot search engines from Matrix Science.

Passive Elution

The passive elution technique allows the extraction of proteins from gel pieces such that they can be re-analysed on the SELDI-TOF. Gel pieces were incubated with 100% acetonitrile for 5 minutes on a shaker, the solution was then removed and the gel pieces left to dehydrate for a short time on a heat block. FAPH solution (50% formic acid, 25% acetonitrile, 15% isopropanol, 10% water) was then added to the gel piece and the sample sonicated for 30 minutes at room temperature. It was then vortexed for a further 2-3 hours, and applied to an NP20 chip to compare the passive elution profile with that of the original sample spectra revealing the biomarker.

4.1 Identification of a 14 kDa Biomarker and Evidence that it is Transthyretin

A 14 kDa biomarker, which was increased upon treatment, was identified on both the IMAC-Cu²⁺ and SAX chips. The Biomarker Wizard data from the analysis of Fraction 4 on the SAX chip at pH9 is shown as well as representative profiles from patients.

FIG. 38: Top half: Biomarker Wizard plot from analysis of fraction 4 from 16 Phase 1b patients on the SAX chip pH9. Only the mass region between 13.5 and 14.6 kDa is shown here. The Day 1 samples prior to start of treatment are shown in blue (u) and the samples from the last day of treatment are shown in red (t). The log normalised intensity plots the log of peak intensity, normalising the average intensity to 0, thereby expressing the difference between sample groups regardless of absolute intensity. Lower half: Representative spectra from two patients showing the appearance of an additional peak following treatment. Statistically significantly higher normalised intensities were observed in the ‘after treatment’ group compared with the ‘before treatment’ group for M/Z 14255 (49.7+/−8.4 vs 20.4+/−3.9; p=0.0000031, Mann-Whitney Utest).

Following analysis on the SELDI-TOF MS, samples from 4 patients were applied to a pH 4-7 immobilised pH gradient strip to separate proteins according to their isoelectric point (charge). After isoelectric focusing, the second dimension separation by size was performed. The gels were stained with Colloidal blue strain to look for differences between the first and last days of treatment. The changes are highlighted in the boxes.

FIG. 39 shows 2D gels of patient plasma. Neat plasma samples were applied to IPG strips pH4-7 to resolve proteins in the first dimension by charge and then in the second dimension by SDS-PAGE to separate by size. Molecular weight size markers are on the left of each gel. The spots of interest lie just between the 14 and 17 kDa markers shown on the left of each gel. The lower gels represent an enlarged view of a further two patients, showing that the change is reproducible.

The top 2 gel spots from each sample were excised and processed in parallel for tryptic digests and passive elution to elute the protein from the gel and permit re-analysis on the SELDI-TOF. The passive elution sample was applied to an NP20 chip to examine the size and profile of the protein peaks to determine whether they are similar to the biomarker peak itself (FIG. 25). A parallel sample was processed for trypsin digestion and peptide mapping, and these spots were identified by the University of Dundee Proteomics facility as Transthyretin, both by MS and MS-MS.

FIG. 40 shows a comparison between the original SELDI-TOF profiles and the passive elution sample extracted from 2D gels. Gels were run in duplicate and the 2 spots at approximately 14 kDa in each sample (Day 1 or Day 10) were excised and processed in parallel for passive elution or trypsin digestion. Passive elution allows the extraction of proteins from gel slices and permits their analysis on the SELDI-TOF-MS. This is shown in the top 4 profiles, which are compared to the original SELDI profiles of these samples (shown in the bottom two spectra). Passive elution of the Day 1 spot 2 did not yield any profile, in agreement with the observation that this spot stains very weakly with the Colloidal stain prior to treatment. Although the passive elution profiles tend to be less well resolved, nevertheless, it does appear that they align perfectly with two of the peaks in the original SELDI profile, as shown by the dashed lines, and the extra spot, present in Day 10, aligns perfectly with the 14255 biomarker identified by the Biomarker Wizard. Tryptic digests were analysed by the University of Dundee.

The two lower spots, which did not appear to change with treatment, were also analysed by tryptic digestion, and were identified as Haptoglobin.

The top two spots in the before and after samples were all identified as Transthyretin. This suggested that the unique spot after treatment was due to posttranslational modifications of Transthyretin, resulting in a more acidic form of the protein, rather than the appearance of a new and different protein. The parallel determination of protein identification and analysis of the sample following passive elution provides unequivocal evidence that the 14 kDa biomarker identified by the Biomarker Wizard is in fact Transthyretin.

The two peaks that correspond to the spots on the 2D gel were approximately 332-341 Da apart. This size difference is indicative of S-palmityl cysteinyl modification of proteins as determined from the Delta Mass Reference Database for Protein Translational Modifications (http://www.abrf.org) suggesting that the additional acidic form of TTR observed following treatment with CYC202 may be due to S-palmityl cysteinylation.

4.2 Identification of a 28 kDa Biomarker Cluster, Apolipoprotein A1

Two statistically significant 28 kDa biomarkers were identified that were present on all 4 chip surfaces that decreased upon treatment. An additional two peaks were observed adjacent to these two peaks that did not reach statistical significance but nevertheless appeared to change reproducibly in response to treatment. The Biomarker Wizard data from the analysis of Fraction 4 on the SAX chip at pH9 is shown in FIG. 26 as well as representative profiles from patients.

Treatment with CYC202 results in a decrease in two peaks at 27923 and 28126, while the peaks at 28292 and 28799, while not reaching statistical significance, nevertheless appear to dramatically increase following treatment.

FIG. 41: Top half: Biomarker Wizard plot from analysis of fraction 4 from 16 Phase 1b patients on the SAX chip pH9. Only the mass region between 26.5 kDa and 30.5 kDa is shown here. The Day 1 samples prior to start of treatment are shown in blue and the samples from the last day of treatment are shown in red. The log normalised intensity plots the log of peak intensity, normalising the average intensity to 0, thereby expressing the difference between sample groups regardless of absolute intensity. Lower half: Representative spectra from two patients showing a decrease in the first two peaks, which correspond to the first two biomarkers, and the appearance or increase in the 3^{rd} peak, which corresponds to the third biomarker, following treatment. Statistically significantly lower normalised intensities were observed in the ‘after treatment’ group compared with the ‘before treatment’ group for M/Z 27923 (31.8+/−13.7 vs 50.6+/−11.8; p=0.00097, Mann-Whitney Utest) and M/Z 28126 (20.4+/−9.9 vs 32.0+/−9.6; p=0.0058, Mann-Whitney U test). The two markers at M/Z 28292 and M/Z 28799 were not deemed to be statistically significant (p=0.95 and 0.19 respectively), although there does appear to be a clear increase in these two patients following treatment.

Following analysis on the SELDI-TOF MS, samples from 4 patients were applied to a pH 4-7 immobilised pH gradient strip to separate proteins according to their isoelectric point (charge). After isoelectric focusing, the second dimension separation by size was performed. The gels were stained with Colloidal blue to look for differences between the first and last days of treatment. The changes are highlighted in the boxes.

FIG. 42 shows 2D gels of patient plasma. Neat plasma samples were applied to IPG strips pH4-7 to resolve proteins in the first dimension by charge and then in the second dimension by SDS-PAGE to separate by size. Molecular weight size markers are on the left of each gel. The spots of interest lie just below the 28 kDa marker.

The images in FIG. 28 represent enlarged views of the 2D gels for a further two patients, showing the appearance of an additional more acidic spot after treatment.

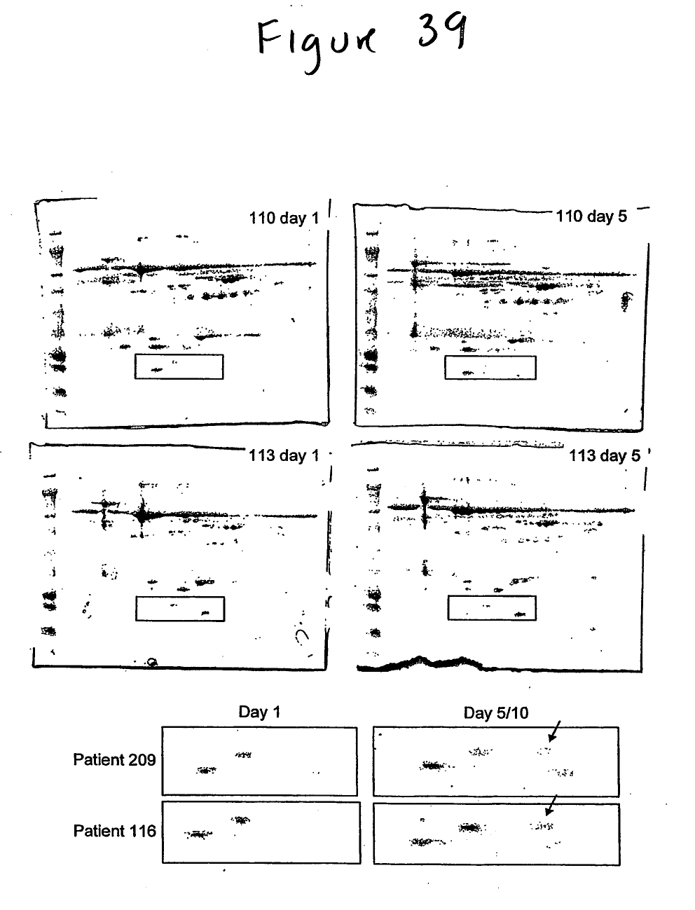

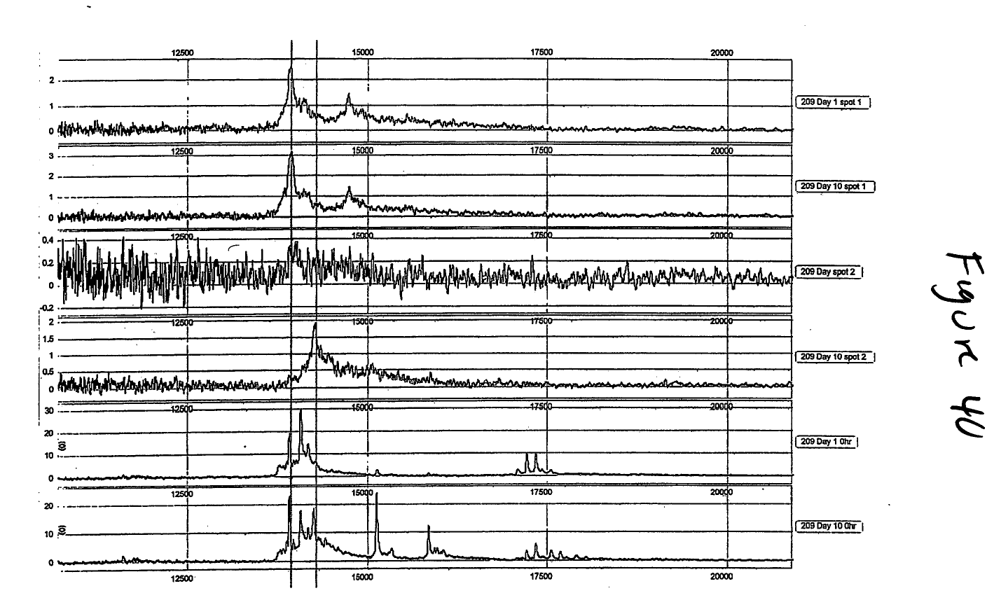

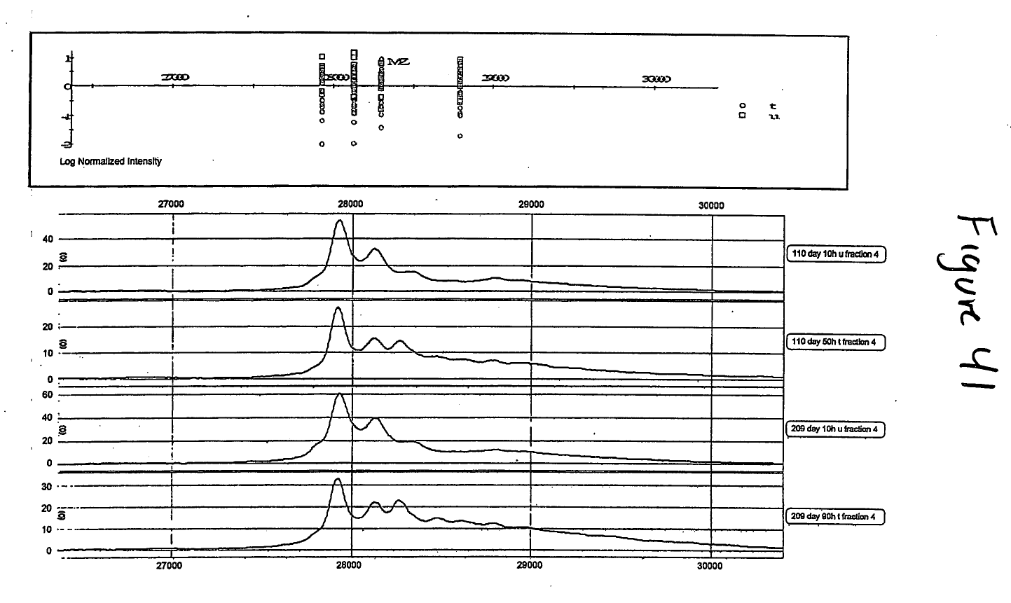

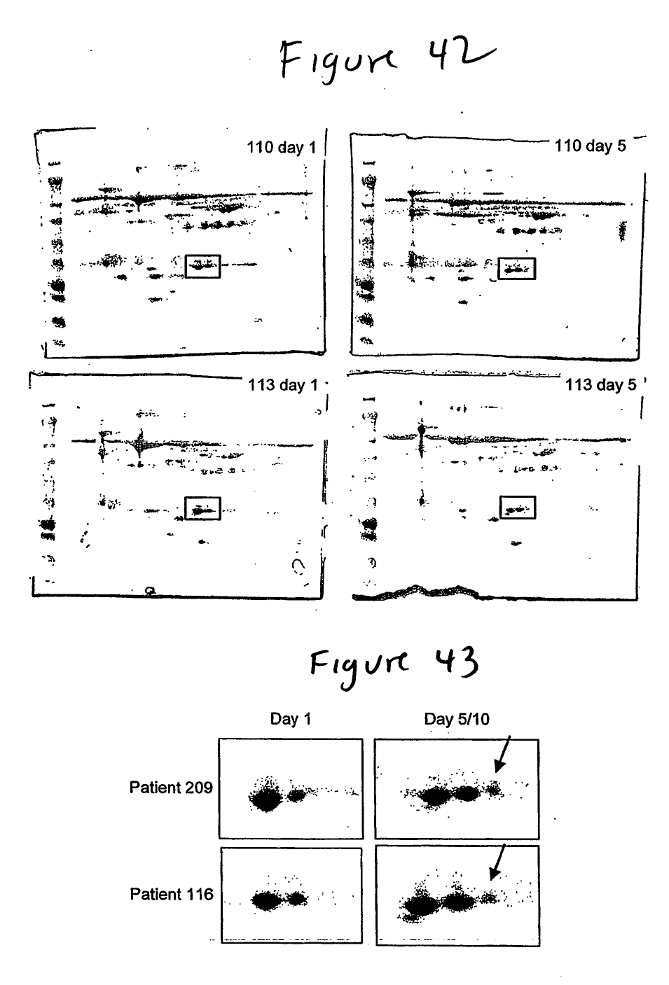

FIG. 43: Enlarged views of 2D gels for patients 209 and 116 indicating the appearance of a third more acidic spot after treatment, and a moderate decrease in the first spot in agreement with the Biomarker Wizard plot.

The sample from patient 209 day 1 was then run on an IPG strip with a wider pH range (pH 3-10) resulting in a single unresolved spot. The gel spot containing this material was excised from the gel and passive elution was performed to extract the protein from the gel piece. This was then applied to an NP20 chip to examine the size and profile of the protein on the SELDI-TOF MS (FIG. 30)

A parallel sample was processed for trypsin digestion and peptide mapping, and was identified by the University of Dundee Proteomics facility as Apolipoprotein A1, both by MS and MS-MS.

The same procedure was also performed on the resolved Apo A1 spots shown above. All spots were identified as ApoA1 suggesting that the unique spot after treatment was due to posttranslational modifications of ApoA1 rather than the appearance of a new and different protein. This most likely corresponded to the 3^{rd} biomarker at 28.292 that did not reach statistical significance across all 16 patients.

FIG. 44:: 2D gel analysis of patient plasma using a pH3-10 IPG strip. The gel was run in duplicate and the spots at 28 kDa were excised and processed in parallel for passive elution or trypsin digestion. Passive elution allows the extraction of proteins from gel slices and permits their analysis on the SELDI-TOF-MS. This is shown in the bottom profile, which compares favourably with the original 209 day 1 sample—the top profile. Tryptic digests were analysed by the University of Dundee.

The parallel determination of protein identification and analysis of the sample following passive elution provides unequivocal evidence that the 28 kDa biomarker identified by the SELDI is in fact Apolipoprotein A1. Therefore, the 28 kDa peaks identified by the Biomarker Wizard as being altered by CYC202 treatment are Apolipoprotein A1 (ApoA1). ApoA1 is subject to post-translational modifications such as glycosylation, acylation and phosphorylation. Deamidated forms of ApoA1 have also been identified.

4.3 Identification of a 7 kDa Biomarker Cluster

A cluster of 7 kDa biomarkers, which appeared only after treatment, were identified on several chip surfaces. The Biomarker Wizard data from the analysis of Fraction 6 on the H50 chip at pH9 and neat plasma on the CM10 chip is shown as well as representative profiles from several patients.

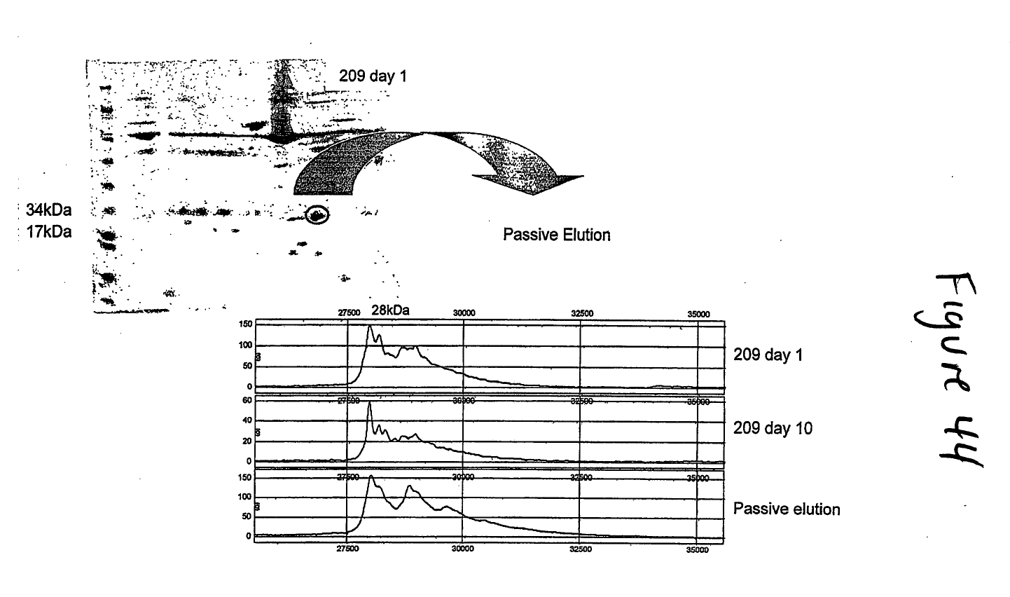

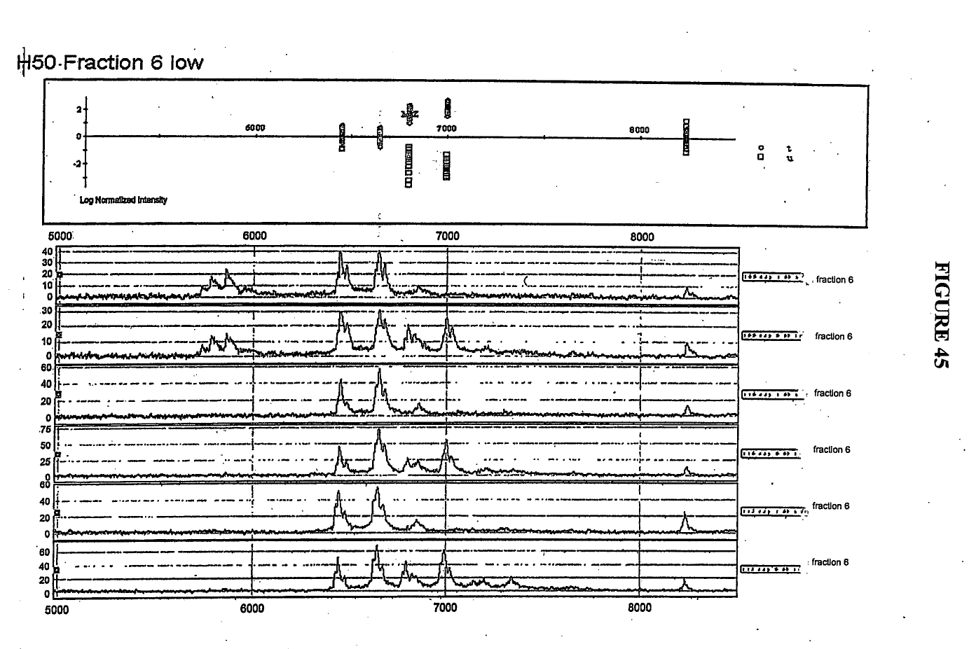

FIG. 45: Top half: Biomarker Wizard plot from analysis of fraction 6 from 16 Phase 1b patients on the H50 chip. Only the mass region between 5 and 8.5 kDa is shown here. The Day 1 samples prior to start of treatment are shown in (u) and the samples from the last day of treatment are shown in (t). The log normalised intensity plots the log of peak intensity, normalising the average intensity to 0, thereby expressing the difference between sample groups regardless of absolute intensity. Lower half: Representative spectra from three patients showing the appearance of an additional two peaks following treatment. Statistically significantly higher normalised intensities were observed in the ‘after treatment’ group compared with the ‘before treatment’ group for M/Z 6799 (2.9+/−1.2 vs 29.1+/−8.1; p=0.0000031, Mann-Whitney U test) and M/Z 6998 (2.1+/−0.7 vs 42.0+/−9.1; p=0.0000031, Mann-Whitney U test)

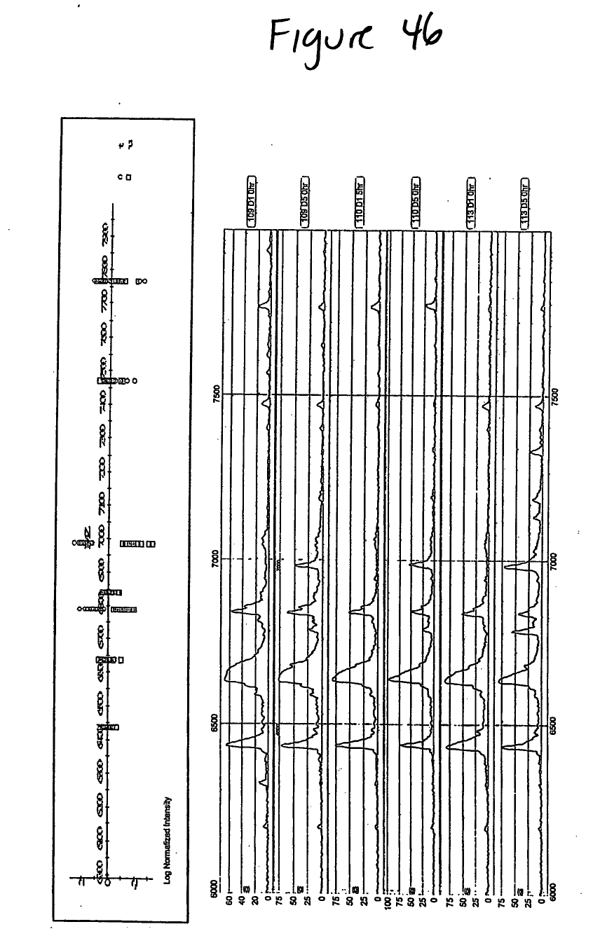

FIG. 46: Top half: Biomarker Wizard plot from analysis of neat plasma from 16 Phase 1b patients on the CM10 chip. Only the mass region between 6 and 8 kDa is shown here. The Day 1 samples prior to start of treatment are shown in blue and the samples from the last day of treatment are shown in red. The log normalised intensity plots the log of peak intensity, normalising the average intensity to 0, thereby expressing the difference between sample groups regardless of absolute intensity. Lower half: Representative spectra from three patients showing the appearance of an additional two peaks following treatment. Statistically significantly higher normalised intensities were observed in the ‘after treatment’ group compared with the ‘before treatment’ group for M/Z 6787 (5.6+/−1.9 vs 27.5+/−10.4; p=0.0000014, Mann-Whitney U test) and M/Z 6984 (3.1+/−1.2 vs 42.2+/−11.8; p=0.0000014, Mann-Whitney Utest)

Following analysis on the SELDI-TOF MS, samples from one patient (209) were applied to a CM10 chip to examine the binding characteristics of the proteins. Binding was performed on all 8 spots at pH3.5, and the chip spots were washed with increasing pH, pH 3.5, 4.5, 5.5 and 7. The biomarker was only present on the chip if it had been washed with pH4.5 or less, suggesting that the approximate pI of the proteins is 4.5-5.

All publications mentioned in the above specification, and references cited in said publications, are herein incorporated by reference. Various modifications and variations of the described methods and system of the present invention will be apparent to those skilled in the art without departing from the scope and spirit of the present invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A method of monitoring activity of a CDKI comprising:
a) isolating a sample from a cell, group of cells, an animal model or human, wherein said cell, group of cells, an animal model or human has been treated with said CDKI;
b) determining altered expression of at least one of i) a gene identified in any of FIGS. 1 to 12; ii) a 28 kDa protein or iii) a 14 kDa protein in said treated sample as compared to an untreated control sample as an indication of said CDKI activity.

2. The method as claimed in claim 1, wherein said altered expression is an increase or decrease of gene expression of a gene identified in any of FIGS. 1 to 12.

3. The method as claimed in claim 2, wherein the gene identified in FIGS. 1 to 12 is selected from ADM, FADD, PAI1, PLAU, PNUTS, TNFSF14, C/EBP alpha, 20585, FUT4, E2F6, 18747, 22147, ZK1, KIAA1698, CCRL2, myc and mcl-1.

4. The method as claimed in any one of claims 1 to 3, wherein the group of cells is a cell culture.

5. The method as claimed in any one of claims 1 to 3, wherein the cells are selected from PBMC, HT29, and A549 cells.

6. The method as claimed in any one of claims 1 to 3, wherein the group of cells is tumor cells, PBMC or lymphocytes.

7. The method as claimed in any one of claims 1 to 3, wherein the sample is blood.

8. The method as claimed in any one of claims 1 to 3, further comprising extracting RNA from said sample and detecting gene expression by QPCR.

9. The method as claimed in any one of claims 1 to 3, wherein the altered expression of at least one of the genes identified in FIGS. 1 to 12 is a decrease in expression compared to the untreated sample.

10. The method as claimed in claim 1, wherein said altered expression is a decrease in a 28 kDa protein.

11. The method as claimed in claim 1, wherein said altered expression is the presence or absence of one or more post translational modifications of a 28 kDa protein or a 14 kDa protein in the treated sample compared to the untreated control sample.

12. The method as claimed in any one of claims 1, 10 and 11 wherein the 28 kDa protein is apolipoprotein A1.

13. The method as claimed in claims 1 or 11, wherein the 14 kDa protein is transthyretin.

14. The method as claimed in any one of claims 1, 10 and 11, wherein the sample is serum, plasma or tissue culture supernatant.

15. The method as claimed in any one of claims 1, 10 and 11, wherein the sample is analysed by protein analysis.

16. The method as claimed in any one of claims 1, 10 and 11, wherein protein analysis is by SELDI-TOF MS or 2-D PAGE.

17. The method as claimed in any one of claims 1, 2, 3, 10 and 11, wherein a CDKI is administered to a mammal.

18. The method as claimed in any one of claims 1, 2, 3, 10 and 11, wherein a CDKI is administered to a human.

19. A method of assessing suitable dose levels of a CDKI comprising monitoring the true altered expression of at least one of the genes identified in FIGS. 1 to 12 after administration of said CDKI to a cell, group of cells, animal model or human.

20. A method of assessing suitable dose levels of a CDKI comprising monitoring altered expression of a 28 or 14 kDa protein after administration of said CDKI to a cell, group of cells, animal model or human.

21. A method as claimed in claim 20, wherein the 28 kDa or 14 kDa protein is a post translationally modified form.

22. A method for identifying a candidate drug having CDKI-like activity comprising administering said candidate drug to a cell, group of cells, animal model or human and detecting altered expression of at least one of i) a gene identified in any of FIGS. 1 to 12; ii) a 28 kDa protein or iii) a 14 kDa protein in said treated sample as compared to an untreated control sample as an indication of CDKI activity.

23. The method as claimed in any one of claims 1, 2, 3, 10, 11, 19, 20, 21 and 22, wherein the CDKI is roscovitine.

24. The method as claimed in claim 23, wherein said roscovitine is R-roscovitine.

25. A method of monitoring the activity of a CDK1, comprising monitoring the altered expression of at least one of the genes as identified in FIGS. 1 to 12 or a gene encoding apolipoprotein A1 or transthyretin.

26. The method of claim 25, wherein the presence of at least one of the genes as identified in FIGS. 1 to 12 or a 28 or 14 kDa protein is monitored after the administration of a CDKI to a cell, group of cells, an animal model or human.

27. The method of claim 26, wherein the CDKI is roscovitine is R-roscovitine.

28. The method of claim 27, wherein said roscovitine is R-roscovitine.

29. A kit for assessing the activity of roscovitine comprising antibodies for a protein encoded by at least one of the genesidentified in FIGS. 1 to 12 or a 28 or 14 kDa protein.

30. A kit for assessing the activity of roscovitine comprising at least one nucleic acid probe wherein said probe is specific for at for at least one of the genes identified in FIGS. 1 to 12.

31. A kit for assessing the activity of roscovitine comprising a QPCR primer having a sequence as set out in FIG. 13 or 14.

32. A method of monitoring the activity of a CDKI comprising:
(i) administering said CDKI to a cell, group of cells, an animal model or human; and
(ii) measuring gene expression in samples derived from the treated and the untreated cells, animal or human; and
(iii) detecting an increase or a decrease in gene expression of at least one of the genes identified in any of FIGS. 1 to 12 in the treated sample as compared to the untreated sample as an indication of CDKI activity.

33. A method of monitoring the activity of roscovitine comprising:
(i) administering roscovitine to a cell, group of cells, an animal model or human; and
(ii) measuring gene expression in samples derived from the treated and the untreated cells, animal or human; and
(iii) detecting an increase or a decrease in gene expression of at least one of the genes identified in FIGS. 1 to 12 in the treated sample as compared to the untreated sample as an indication of roscovitine activity.

34. The method as claimed in claim 33 wherein the gene identified in FIGS. 1 to 12 is selected from ADM, FADD, PAI1, PLAU, PNUTS, TNFSF14, C/EBP alpha, 20585, FUT4, E2F6, 18747, 22147, ZK1, KIAA1698, CCRL2, myc and mcl-1.

35. The method according to claim 33, wherein roscovitine is administered to a mammal.

36. The method according to any one of claims 33 to 35, wherein roscovitine is administered to a human.

37. The method according to claim 33 or claim 34, wherein the group of cells is a cell culture.

38. The method according to claim 37, wherein the cells are selected from PBMC, HT29, and A549 cells.

39. The method according to any one of claims 1, 2, 3, 10, 11, 19, 20, 21, 22, 25, 26, 27, 33, 34 and 35, wherein the presence of at least one of the genes identified in FIGS. 1 to 12 is detected in tumor cells or lymphocytes.

40. The method according to any one of claims 1, 2, 3, 10, 11, 19, 20, 21, 22, 25, 26, 27, 33, 34 and 35, wherein the level of at least one of the genes identified in FIGS. 1 to 12 is less than that detected prior to administration of roscovitine.

41. A method of assessing suitable dose levels of roscovitine comprising monitoring the degree and rate of expression of at least one of the genes identified in FIGS. 1 to 12 after administration of roscovitine to a cell, group of cells, animal model or human.

42. A method of identifying a candidate drug having roscovitine-like activity comprising administering said candidate drug to cell, group of cells, animal model or human and monitoring the presence or absence of at least one of the genes as identified in FIGS. 1 to 12.

43. The method of claims according to any one of claims 33, 34, 35, 41 and 42, wherein roscovitine is R-roscovitine.

44. The method of claim 25, wherein said genes comprise at least one of the genes as identified in FIGS. 1 to 12.

45. The mthod of claim 44, further comprising monitoring for the presence of at least one of the genes as identified in FIGS. 1 to 12, after the administration of roscovitine to a cell, group of cells, an animal model or human.

46. The method to claim 44 or 45, wherein roscovitine is R-roscovitine.

47. A kit for assessing the activity of roscovitine comprising antibodies for at least one of at least one of the genes as identified in FIGS. 1 to 12.

48. A kit for assessing the activity of roscovitine comprising a nucleic acid probe for at least one of the genes identified in FIGS. 1 to 12.
